(19) United States
(12) Patent Application Publication
(10) Pub. No.: US 2015/0328329 A1
(43) Pub. Date: Nov. 19, 2015

(54) LIGANDS THAT TARGET HCV-E2 BINDING SITES ON CD81 AND THERAPEUTIC METHODS USING THEM

(71) Applicant: American University of Cairo, New Cairo (EG)

(72) Inventors: Hassan AZZAZY, Alexandria (EG); Reem Al-Olaby, Cairo (EG); Rodney Balhorn, Livermore, CA (US)

(73) Assignee: American University of Cairo, New Cairo (EG)

(21) Appl. No.: 14/717,635

(22) Filed: May 20, 2015

Related U.S. Application Data
(63) Continuation of application No. PCT/US2013/071056, filed on Nov. 20, 2013.
(60) Provisional application No. 61/728,486, filed on Nov. 20, 2012.

(51) Int. Cl. A61K 47/48; A61K 31/513; A61K 31/522; C07D 519/00; C07D 495/04

(52) U.S. Cl. CPC A61K 47/48061; C07D 519/00; C07D 495/04; A61K 31/522; A61K 31/513

(57) ABSTRACT

Ligands that target the HCV-E2 binding site and methods of making and using them. A series of ligand binding sites on the large extracellular loop of the open conformation of CD81 have been identified. Several important sites were located in regions identified by mutational studies to be the site of E2 binding. Ligands that recognize these sites were identified. Linking together two or three ligands that bind with low or moderate affinities to different structurally unique sites on a target protein were used to generate small molecule ligand conjugates that exhibit very high affinities to their CD81 targets. Hybrid ligand molecules were also designed using fragment-based drug design methods to generate analogs of the ligands that bind more tightly to the protein than the parent compounds. Identification and design of groups of compounds that bind to CD81 for use as therapeutics for treating patients infected by Hepatitis C virus and other viruses that interact with CD81. By binding to CD81, these molecules can block 1) HCV and other viral entry into cells (infection), 2) inflammatory responses caused by HCV and other viral infections, and 3) the induction of HCV associated cancers.

### CROSS-REFERENCE TO RELATED APPLICATIONS

This application claims priority under 35 U.S.C. 120 to PCT/US2013/071056, filed Nov. 20, 2013, which claims priority to U.S. Provisional Application No. 61/728,486, filed Nov. 20, 2012, the contents of which are incorporated by reference in their entireties.

### BACKGROUND OF THE INVENTION

1. Field of the Invention

Small molecule ligands that bind to sites on the large extracellular loop of human CD81 were identified using structure-based methods. Compounds that block or interfere with attachment, invasion and infection of cells by Hepatitis C Virus (“HCV”) were produced and identified by linking together two or more of these ligands. Such compounds can bind to different sites on CD81 and exhibit more specificity and a higher affinity for CD81. These ligands and ligand conjugates can inhibit binding of the HCV E protein, a major viral protein involved in the attachment of HCV to cells that express CD81, such as human hepatocytes.

2. Description of the Related Art

Hepatitis C virus (HCV) is a global health problem. The virus is a blood borne pathogen that is transmitted mainly through nosocomial infections, blood transfusions, dental procedures and haemodialysis. During HCV replication, the post-translational processing and cleavage of the virus polyprotein produces ten structural and non-structural proteins. The crystal structures that have been determined for a number of these proteins are being used to facilitate drug and vaccine development [2-9].

The amino acid sequence of the E2 protein has regions in which the amino acid sequence can vary from one strain of virus to another.

Albecka et al. identified functional and structural regions in HCV envelope glycoprotein E2. Specifically, domain I in HCV E2 (formed of 2 units DIa and DIb) was reported to contain determinants essential for interaction with CD81-LEL. The 2 parts of Domain I were reported to interact together to form the CD81-LEL binding region in HCV E2. FIG. 3 of Albecka describes a study that was done to determine the significance of Domain I in CD81-LEL: HCV E2 interaction. The teachings in Albecka pertaining to how HCV interacts with CD81 are incorporated by reference.

FIG. 3 of Krey et al., The Disulfide Bonds in Glycoprotein E2 of Hepatitis C Virus Reveal the Tertiary Organization of the Molecule. PLoS Pathog 6(2): e1000762; doi:10.1371/journal.ppat.1000762 (2010) compares the amino acid sequences for E2 from 9 HCV genotypes and marks with blue circles amino acid residues interacting with CD81. The structural and structure-function information in Krey pertinent to how HCV interacts with CD81 is incorporated by reference.

Several cell surface receptors have been suggested to play a role in HCV entry into hepatocytes [10]. These include LDL-R, heparan sulphate [11], scavenger receptor class BI (SR-BI) and CD81 [12,13]. Pileri et al. was the first to identify CD81, a 26 kDa protein that belongs to the tetraspanins super family, as an important HCV receptor [14]. While this protein mediates the invasion of hepatocytes by HCV, it is also widely expressed in both lymphoid and non-lymphoid tissues. CD81 contains six structural domains, four of which are transmembrane domains and two of which are hydrophilic extracellular domains that make up the large and small extracellular loops [15]. The amino acid sequence and many structural features of CD81 are known and representative structures are described by NCBI Reference Sequence NP_{—}004347.1 and by SEQ ID NOS: 2 and 3.

CD81 has been linked to a number of biological processes. These include viral attachment and entry into cells that express CD81 (including hematopoetic, lymphoid, endothelial and epithelial cells) and invasion of the liver by Plasmodium falciparum. CD81 is associated with cellular proliferation, growth regulation, response to wounding and wound healing, and various other immunological and cellular responses. Human immunodeficiency virus infection is also mediated by CD81; Gordon Alonzo, et al., Tetraspanins CD9 and CD81 modulate HIV-1-induced membrane fusion, J. Immunol. 2006 Oct. 15; 177(8):5129-37.

One reason CD81 has become such an important target for drug development is because the large extracellular loop of CD81 (CD81-LEL) has been shown to bind to the HCV E2 glycoprotein [16-19]. Zhang et al. discovered that CD81-LEL is also important for efficient replication of the HCV genome [18]. In addition, the E2:CD81-LEL interaction has been reported to induce several immuno-modulatory effects, including a co-stimulatory signal in naive and antigen-experienced T cells in vitro that leads to production of the pro-inflammatory cytokine γ-interferon. This suggests that the E2:CD81-LEL interaction plays a role in T-cell-mediated liver inflammation and may contribute to liver damage. The interaction of these two proteins also appears to down regulate T-cell receptors and suppress the activity of natural killer cells [18].

CD81 is an important target for designing new anti-HCV therapeutics because of its known participation in viral invasion of cells and in processes that cause liver damage. Some of the first inhibitors designed to block the E2:CD81-LEL interaction were CD81 mimics developed by Van Compernolle et al. [20]. Small molecules were designed to mimic the solvent exposed hydrophobic ridge of helix D in the CD81-LEL domain and were found to bind HCV E2 reversibly and to competitively block the binding of E2 to CD81 [20]. This was the first direct demonstration that CD81 is an important receptor in HCV entry [20]. In addition, the mutational studies conducted by Higginbottom et al. [17] and Drummer et al. [19] identified the key amino acid residues that contribute to the E2:CD81-LEL interaction. Kitadokoro et al. determined the 3D structure of CD81-LEL using X-ray crystallography [21,22]. In this structure the C and D helices form a cleft-like motif within the E2 binding site, a large cavity considered to be an excellent target site for inhibitor development.
Compernolle et al. [20] described molecules that mimic portions of the CD81 structure that inhibited HCV binding to CD81, but they did not report ligands that bind to CD81 and inhibit HCV binding. Holzer, et al. described benzyl salicylate as a moderate inhibitor of HCV binding to CD81. Benzyl salicylate is a recognized allergen having undesirable effects such as increasing the proliferation of breast cancer cells in vitro, Charles, A. K, et al., J. Appl. Toxicol. 29(5):422-34 (2009).
Bolognesi, et al., U.S. Pat. No. 7,657,385 describes structure-based design of compounds which bind to CD81 for the purpose of blocking HCV binding.
Balhorn, et al. [23] and DeNardo [24] describe tridentate antibody-mimics that exhibit 1000-fold higher affinities for their specific targets compared to bidentate antibody mimics containing two ligands.

As apparent from the background art, there is an on-going and urgent need to discover new molecules that safely, selectively and effectively inhibit the binding of HCV to human cells in order to prevent, ameliorate or treat HCV infection.

As described herein the inventors have identified a group of new molecules that bind to specific segments of CD81 as well as novel hybrid or linked molecules having an even higher affinity for CD81 than individual ligands.

### BRIEF SUMMARY OF THE INVENTION

Using computational docking and virtual screening methods, the inventors have identified a group of small organic molecules—ligands for CD81—that bind to different sites on CD81. Different subsets of these molecules respectively bind to five different cavities located within the E2 binding site on CD81-LEL. Several molecules have been tested and shown to block HCV E2 protein binding to CD81 in vitro.

Following the experimental verification of binding of the ligands to a recombinant form of CD81-LEL using SPR and DPI, prototype ligand conjugates were designed by linking together pairs of ligands that have been determined to bind to bind to different sites on the CD81 protein [23, 24].

One aspect of the invention is directed to the small molecules discovered to bind to these different sites on CD81. These include the molecules described in the tables below. Assays that help validate that these small molecule ligands will inhibit the binding of HCV and other pathogens to CD81 or modulate CD81 interaction with other ligands or receptors are also disclosed. Due to variations in the HCV E2 amino acid sequence between different HCV strains some small molecule ligands are likely to inhibit some strains to a greater or lesser degree than other strains depending whether the variations are in a portion of E2 interacting with a CD81 binding site as described herein. These differences provide a way to identify and select a small molecular ligand that preferentially inhibits the binding of a particular strain to CD81. A particular small molecule inhibitor can be customized or selected to treat infection by a particular HCV or other microbial strain, for example, by selecting one that preferentially inhibits that strain so that the effective dosage administered to a subject is reduced and the subject experiences reduced side-effects or drug toxicity. Alternatively, to provide a broader antiviral spectrum, cocktails of different small molecule inhibitors can be produced that include small molecule inhibitors that modulate or block binding of a variety of HCV strains; especially those endemic in a particular geographical area or population segment.

Another aspect of the invention is the development of selective ligand conjugates that bind to CD81 with higher affinities than the individual ligands used to create the conjugates. These ligand conjugates will bind to two or more sites on CD81. Each conjugate comprises two or more small molecule ligands for CD81 linked together. Examples of ligands that can be linked to form a ligand conjugate that binds CD81 are shown in Table 1.

Assays are also contemplated to validate the use of the individual ligands, structural analogs of the ligands, and ligand conjugates, to inhibit the binding of HCV and other pathogens to CD81 or to modulate CD81 interaction with other ligands or receptors.

Similarly, the invention encompasses more complex ligand conjugates that bind to three, four or more sites on CD81 and methods of using them to inhibit the binding of HCV and other pathogens to CD81 or to modulate CD81 interaction with other ligands or receptors.

Hybrid molecules can be engineered based on the chemical features of the individual ligands such as those described by Table 1. Examples of these hybrid molecules are shown in Table 2 and representative chemical structures appear in FIG. 16.

The inventors identified five ligand binding sites on CD81 as shown in FIGS. 9-13. These sites are also characterized by neighboring amino acids known to participate in HCV E2 binding, CD81 amino acid residues that surround the sites, and/or by the binding of particular ligands to these sites.

Site 1. This site is located near CD81 amino acid residues 163, 182, 184, 186 and 188 that are important for HCV E2 binding to CD81. Ligand 5069 is an example of a ligand that binds to this site. The site is a cavity on the surface of the protein surrounded by the following amino acid residues: Ser160, Thr163, Ala164, Thr167, Ile181, Leu185, Glu188 and Gln192, see FIG. 9.

Site 2. This site is located near CD81 amino acid residues 162, 163 and 182 that are important for HCV E2 binding to CD81. Ligand 73735 is an example of a ligand that binds to this site. The site is a cavity on the surface of the protein surrounded by the following amino acid residues: Val136, Asp138, Leu165, Thr167, Lys171, Asn173, Ser177, Asn180 and Ile181, see FIG. 10.

Site 3. This site is located near CD81 amino acid residues 182, 186, 188, 196 that are important for HCV E2 binding to CD81. Ligand 93033 is an example of a ligand that binds to this site. The site is a cavity on the surface of the protein surrounded by the following amino acid residues: Glu152, Ser177, Asn180, Phe186, Glu188 and His202, see FIG. 11.

Site 4. This site is located near CD81 amino acid residue 155 that is important for HCV E2 binding to CD81. Ligand 81750 is an example of a ligand that binds to this site. The site is a cavity on the surface of the protein surrounded by the following amino acid residues: Lys148, Glu152, Leu170, Asn173, Ser177, Asn180, and Lys301, see FIG. 12.

Site 5. This site is located near CD81 amino acid residue 162 that is important for HCV E2 binding to CD81. Ligand 68982 is an example of a ligand that binds to this site. The site is a cavity on the surface of the protein surrounded by the following amino acid residues: Asp117, Lys121, Glu125, Gln132, Ser160, Thr161, Gln192 and Asp195, see FIG. 13.

The ligands disclosed herein find many applications including as antiviral compounds that modulate or interfere with virus binding to cells expressing CD81, as competitive inhibitors of virus binding in vivo, in vitro, or in antivirus compositions, such as disinfectants or virus-neutralizing compositions; or as reagents or tools for identifying new ligands that bind to CD81 or the CD81 sites described above. For example, ligands identified by the inventors can be used in a competitive inhibition assay where competitive binding of a known CD81 ligand and a putative ligand are determined for CD81 or for the specific CD81 sites described above.

Chemical derivatives of the CD81 ligands described herein are contemplated including ligands chemically derivatized to remove undesirable physical or biological properties or to modulate absorption, distribution, or localization of the derivatized ligand. Prodrugs of the ligands disclosed, which lack biological activity until transformed in vivo or in vitro into a ligand as described herein, are also contemplated. Methods for derivatizing a chemical ligand or producing a prodrug from it are known.

Specific embodiments of the invention include the following:
1. A molecule that binds to at least one of Sites 1, 2, 3, 4, or 5 on CD81 or that inhibits the binding of a molecule known to bind to at least one of Sites 1, 2, 3, 4, or 5 to the site.
2. The molecule of embodiment 1 that binds to at least one of Sites 1, 2, 3, 4 or 5.
3. The molecule of embodiment 1 that inhibits the binding of a molecule known to bind to at least one of Sites 1, 2, 3, 4, or 5.
4. The molecule of embodiment 1 that is a small organic molecule.
5. The molecule of embodiment 1 that binds to Site 1 on CD81 selected from the group consisting of 5069, 7436, 7962, 16646, 21034, 23895, 30930, 31712, 73170, 94914, 97538, 98026, 106963, 117922, 120631, 123115, 134137, 144958, 153172, 164965, 165665, 252359, and 689002; wherein Site 1 comprises a cavity on the protein surface bounded by CD81 amino acids Ser160, Thr163, Ala164, Thr167, Ile181, Leu185, Glu188 and Gln192. Molecules which block or inhibit the binding of those described in the group above to CD81, especially to CD81 Site 1.
6. The molecule of embodiment 1 that binds to Site 2 on CD81 selected from the group consisting of molecule 38743, 156957, 127947, 73735, 55573, 41066, 11891, 63865, 408860, 362639, 36914, 23895, and 403374; wherein Site 2 comprises a cavity on the protein surface bounded by amino acids Val136, Asp138, Leu165, Thr167, Lys171, Asn173, Ser177, Asn180 and Ile181. Molecules which block or inhibit the binding of those described in the group above to CD81, especially to CD81 Site 2.
7. The molecule of embodiment 1 that binds to Site 3 on CD81 selected from the group consisting of molecule 93033, 80807, 25368, 25678, 60239, 75866, 87504, 331931, 20586, 403374, 8481, and 5856; wherein Site 3 comprises a cavity on the protein surface bounded by amino acids Glu152, Ser177, Asn180, Phe186, Glu188 and His202. Molecules which block or inhibit the binding of those described in the group above to CD81, especially to CD81 Site 3.
8. The molecule of embodiment 1 that binds to Site 4 on CD81 selected from the group consisting of molecules 16631, 40614, 68971, 78623, 81750, 401077, 408734, 303800, 75846, 638134, 70980, 89720, 25678, 215276, 16162 and 60239; wherein Site 4 comprises a cavity on the protein surface surrounding amino acid Ser177 bounded by amino acids Glu152, Ser177, Lys148, Leu170, Asn173, Asn180, and Lys301. Molecules which block or inhibit the binding of those described in the group above to CD81, especially to CD81 Site 4.
9. The molecule of embodiment 1 that binds to Site 5 on CD81 that is molecule 68982; 75866-148832, 601359 and 142446; wherein Site 5 comprises a cavity on the protein surface bounded by amino acids Asp117, Lys121, Glu125, Gln132, Ser160, Thr161, Gln192 and Asp195. Molecules which block or inhibit the binding of those described in the group above to CD81, especially to CD81 Site 5.
10. The molecule of embodiment 1 that is selected from the group consisting of molecules 75866, 87504, 25678. 40614, 134137, 7436, 117922, 144958, 68982, and 75846.
11. The molecule of embodiment 1 that is selected from the group of molecules described by Table 1.
12. The molecule of embodiment 1 that is selected from the group of molecules described by Table 2.
13. The molecule of embodiment 1 that is selected from the group of molecules described by Table 3.
14. The molecule of embodiment 1 that is selected from the group of molecules described by Table 4.
15. The molecule of embodiment 1 that is selected from the group of molecules described by Table 5.
16. The molecule of embodiment 1 that is selected from the group of molecules described by Table 6.
17. The molecule of embodiment 1 that is selected from the group of molecules described by Table 7.
18. The molecule of embodiment 1 that is selected from the group of molecules described by Table 8.
19. The molecule of embodiment 1 that is selected from the group of molecules described by Table 9.
20. The molecule of embodiment 1 that is selected from the group of molecules described by Table 10.
21. The molecule of embodiment 1 that is selected from the group of molecules described by Table 11.
22. The molecule of embodiment 1 that is selected from the group of molecules described by Table 12.
23. The molecule of embodiment 1 that is selected from the group of molecules described by Table 13.
24. The molecule of embodiment 1 that is selected from the group of molecules described by Table 14.
25. The molecule of embodiment 1 that is selected from the group of molecules described by Table 15.
26. The molecule of embodiment 1 that is selected from the group of molecules described by Table 16.
27. The molecule of embodiment 1 that is selected from the group of molecules described by Table 17.
28. The molecule of embodiment 1 that is selected from the group of molecules described by Table 18.
29. The molecule of embodiment 1 that is selected from the group of molecules described by Table 19.
30. The molecule of embodiment 1 that is selected from the group of molecules described by Table 20.
31. The molecule of embodiment 1 that is selected from the group of molecules described by Table 21.
32. The molecule of embodiment 1 that is selected from the group of molecules described by Table 22.
33. The molecule of embodiment 1 that is selected from the group of molecules described by Table 23.
34. The molecule of embodiment 1 that is selected from the group of molecules described by Table 24.
35. The molecule of embodiment 1 that is selected from the group of molecules described by Table 25.
36. The molecule of embodiment 1 that is selected from the group of molecules described by Table 26.
37. The molecule of embodiment 1 that is selected from the group of molecules described by Table 27.
38. The molecule of embodiment 1 that is selected from the group of molecules described by Table 28.
39. The molecule of embodiment 1 that is selected from the group of molecules described by Table 29.
40. The molecule of embodiment 1 that is selected from the group of molecules described by Table 30.
41. The molecule of embodiment 1 that is selected from the group of molecules described by Table 31.
42. The molecule of embodiment 1 that is selected from the group of molecules described by Table 32.
43. The molecule of embodiment 1 that is selected from the group of molecules described by Table 33.
44. The molecule of embodiment 1 that is selected from the group of molecules described by Table 34.
45. The molecule of embodiment 1 that is selected from the group of molecules described by Table 35.
46. The molecule of embodiment 1 that is selected from the group of molecules described by Table 36.
47. The molecule of embodiment 1 or a conjugate thereof that binds to a region of CD81 to which a virus, pathogen, or other CD81 ligand binds.
48. The molecule of embodiment 1 or a conjugate thereof that binds to a region of CD81 to which a viral protein, glycoprotein, carbohydrate or other determinant binds.
49. The molecule of embodiment 1 or a conjugate thereof that inhibits or prevents a virus or other CD81 ligand from interacting with CD81.
50. The molecule of embodiment 1 or a conjugate thereof that reduces or blocks the ability of a virus to invade, infect or re-infect the cell expressing or carrying CD81.
51. The molecule of embodiment 1 or a conjugate thereof that is covalently attached to or non-covalently associated with an effector molecule, such as those selected from the group consisting of a dendrimer, nanoparticle, liposome, biotin, avidin, avidin analog, antibody, protein, carbohydrate, lipid, other bulky molecule.
52. A composition comprising at least one molecule according to embodiment 1 or a conjugate thereof and a pharmaceutically acceptable carrier or excipient. The composition may comprise a cocktail of two, three, four, five, six, seven, eight, nine or ten or more ligands according to embodiment 1 or conjugates thereof.
53. The composition of embodiment 52 in a unit dosage form.
54. The composition of embodiment 52 in a form suitable for administration to a human.
55. A conjugate comprising at least two, three, four, five, six, seven, eight, nine, ten or more molecules as described by embodiment 1, which bind to the same or different sites on CD81. Advantageously, different molecules constituting the conjugate will bind to different sites on CD81, wherein the different sites are Sites 1, 2, 3, 4 or 5. These conjugates may optionally contain a spacer or linker covalently linking the molecules that form them. Other moieties may also be present such as effectors or tags.
56. A covalent conjugate comprising a molecule of embodiment 1 and at least one other molecule.
57. The conjugate of embodiment 55 that comprises two molecules, which each bind to a different site on CD81; and optionally a spacer or linker between the two molecules.
58. The conjugate of embodiment 55 that comprises at least three molecules, which each bind to a different site on CD81; and optionally a spacer or linker between the at least three molecules.
59. The conjugate of embodiment 55 that comprises three molecules, which each binds to a different site on CD81, that is selected from the group consisting of molecules described by Tables 32, 33, 34, 35, 36, and 37; and optionally a spacer or linker between the at least three molecules.
60. A conjugate of at least two, three, four, five, six, seven, eight, nine, ten or more molecules according to embodiment 1 that are connected via bonds or chemical linkers or spacers. A covalent conjugate comprising two molecules of embodiment 1 and optionally a spacer or linker attaching them. A covalent conjugate comprising three molecules of embodiment 1 and optionally covalent linkers or spacers attaching them.
61. The conjugate of embodiment 55 that is selected from the group consisting of 25678-lys-lys-75846, 40614-lys-lys-75846, 117922-lys-lys-75866, 75866-lys-lys-68982, 75866-lys-lys-144958, and 40614-lys-lys-25678.
62. The covalent conjugate of embodiment 55 that comprises a chemical linker or spacer that ranges in length from 0 to 3 nm.
63. The conjugate of embodiment 55, wherein the chemical linker is selected from the group consisting of a chemical bond, a bivalent hydrocarbon radical, a multivalent hydrocarbon radical, a bivalent hydrocarbon radical containing at least one heteroatom, a multivalent hydrocarbon radical containing at least one heteroatom, a multivalent radical containing oxygen, nitrogen or sulfur. These may include connectors comprised of one or more molecules or polymers selected from a group consisting of other organic molecules, polyethylene glycol or its functionalized derivatives, amino acids, peptides, peptide analogs, sugars or carbohydrates, nucleic acids, nucleic acid analogs, straight chain carbons, heterocycles, branched carbon chains, thiols, dendrimers, or other molecular components or subunits that can be used to connect two molecules.
64. The conjugate of embodiment 55, wherein the chemical linker is a peptide or peptide analog, a carbohydrate or carbohydrate analog, a sugar or sugar analog, nucleic acid or nucleic acid analog, or a dendrimer.
65. The conjugate of embodiment 55 comprising two ligands that bind to different sites on CD81, where the different sites are CD81 Sites 1, 2, 3, 4 or 5. The conjugate of embodiment 55 comprising three ligands that bind to different sites on CD81, where the different sites are CD81 Sites 1, 2, 3, 4 or 5.
66. An organic small molecule created by connecting 689002 to one of the molecules listed in Table 3.
67. An organic small molecule created by connecting 30930 to one of the molecules listed in Table 4.
68. An organic small molecule created by connecting 165665 to one of the molecules listed in Table 5.
69. An organic small molecule created by connecting 93033 to one of the molecules listed in Table 6.
70. An organic small molecule created by connecting 16631 to one of the molecules listed in Table 7.
71. An organic small molecule created by connecting 63865 to one of the molecules listed in Table 8.
72. An organic small molecule created by connecting 5069 to one of the molecules listed in Table 9.
73. An organic small molecule created by connecting 11891 to one of the molecules listed in Table 10.
74. An organic small molecule created by connecting 21034 to one of the molecules listed in Table 11.
75. An organic small molecule created by connecting 41066 to one of the molecules listed in Table 12.
76. An organic small molecule created by connecting 55573 to one of the molecules listed in Table 13.
77. An organic small molecule created by connecting 68971 to one of the molecules listed in Table 14.
78. An organic small molecule created by connecting 68982 to one of the molecules listed in Table 15.
79. An organic small molecule created by connecting 73735 to one of the molecules listed in Table 16.
80. An organic small molecule created by connecting 75846 to one of the molecules listed in Table 17.
81. An organic small molecule created by connecting 78623 to one of the molecules listed in Table 18.
82. An organic small molecule created by connecting 81750 to one of the molecules listed in Table 19.
83. An organic small molecule created by connecting 98026 to one of the molecules listed in Table 20.
84. An organic small molecule created by connecting 127947 to one of the molecules listed in Table 21.
85. An organic small molecule created by connecting 156957 to one of the molecules listed in Table 22.
86. An organic small molecule created by connecting 401077 to one of the molecules listed in Table 23.
87. An organic small molecule created by connecting 408734 to one of the molecules listed in Table 24.
88. An organic small molecule created by connecting 303800 to one of the molecules listed in Table 25.
89. An organic small molecule created by connecting 38743 to one of the molecules listed in Table 26.
90. An organic small molecule created by connecting 408860 to one of the molecules listed in Table 27.
91. An organic small molecule created by connecting 362639 to one of the molecules listed in Table 28.
92. An organic small molecule created by connecting 123115 to one of the molecules listed in Table 29.
93. An organic small molecule created by connecting 70980 to one of the molecules listed in Table 30.
94. An organic small molecule created by connecting 36914 to one of the molecules listed in Table 31.
95. An organic small molecule created by connecting 25368 to 638134.
96. The molecule of embodiment 55 that is a ligand conjugate that binds to at least three of Sites 1, 2, 3, 4 or 5.
97. The molecule of embodiment 55 that is a small organic molecule comprising three different molecules that are linked together, each selected from a different group that binds to a different site on CD81, wherein said groups are selected from those of Tables 32, 33, 34, 35, 36, and 37.
98. The molecule of embodiment 55 that is a hybrid molecule that combines and comprises chemical moieties of two or more molecules that each bind to the same site at adjacent positions or at positions that overlap; wherein said combined chemical moieties are those moieties that bind to the CD81 site; and wherein said site is Site 1, 2, 3, 4 or 5.
99. The conjugate of embodiment 55 that is covalently attached to or non-covalently associated with an effector molecule, such as those selected from the group consisting of a dendrimer, nanoparticle, liposome, biotin, avidin, avidin analog, antibody, protein, carbohydrate, lipid or other effector molecule.
100. A composition comprising at least one conjugate according to embodiment 55 and a pharmaceutically acceptable carrier or excipient, optionally in a unit dose or in a form suitable for administration to a human. The composition may comprise a cocktail of two, three, four, five, six, seven, eight, nine or ten or more conjugates according to embodiment 55.
101. A method for modulating a biological activity of CD81 or an activity mediated by or through CD81 comprising contacting CD81 with at least one molecule of embodiment 1 or a conjugate according to embodiment 55. The molecule of embodiment 1 or conjugate of embodiment 55 may bind to a region of CD81 to which viral proteins bind, to a region of CD81 to which viruses bind, may inhibit or prevent the virus from interacting with CD81, or may reduce or block the ability of the virus to invade, infect or re-infect the cell containing CD81.
102. The method of embodiment 101 that inhibits the binding of a pathogen that binds to CD81 comprising contacting CD81 with at least one molecule of embodiment 1 or at least one conjugate of embodiment 55, or both.
103. The method of embodiment 101, wherein said pathogen is HCV, HIV, Plasmodium or other pathogen that utilizes CD81 to attach to or infect cells.
104. The method of embodiment 101, wherein said pathogen is a cell infected with HCV.
105. The method of embodiment 101, wherein said pathogen is a cell or microorganism that expresses or comprises a HCV E2 polypeptide, a fragment of a HCV E2 polypeptide, or an analog of a HCV polypeptide having at least 80%, 95% or 99% similarity to the amino acid sequence described by Uniprot C4MR37 (SEQ ID NO: 1).
106. The method of embodiment 101 that inhibits HCV, HIV, or another virus from invading cells, comprising contacting CD81 with at least one small organic molecule according to embodiment 1 or conjugate of embodiment 55 to CD81, thus inhibiting or blocking virus or viral protein attachment to CD81 or thus inhibiting virus or viral protein interaction with CD81.
107. The method of embodiment 101 that inhibits HCV or another virus from infecting, re-infecting or damaging liver cells, comprising contacting and binding at least one small organic molecule according to embodiment 1 or conjugate according to embodiment 55 to CD81, thus inhibiting or blocking virus or virus protein attachment to CD81 or virus or virus protein interaction with CD81.
108. The method of embodiment 101 that inhibits HCV, HIV or another virus from invading cells, comprising attaching at least one small organic molecule of embodiment 1 or the conjugate of embodiment 55 to an effector that improves the efficiency of the small organic molecule for blocking virus or viral protein attachment to, or blocking virus or viral protein interaction with, CD81.
109. The method of embodiment 101 that inhibits HCV or another virus from infecting, re-infecting or damaging liver cells, comprising attaching at least one small organic molecule of embodiment 1 or conjugate according to embodiment 55 to an effector that improves the ability of the small molecule to block virus or virus protein attachment to or interaction with CD81 and minimizes or prevents liver damage.
110. The method of embodiment 101 that inhibits pro-inflammatory cytokine production, comprising contacting CD81 with at least one small organic molecule of embodiment 1 or conjugate of embodiment 55 to block or reduce the interaction of a virus CD81 ligand or another CD81 ligand with CD81 or cells containing or expressing CD81.
111. The method of embodiment 101 that inhibits liver inflammation, comprising contacting at least one small organic molecule of embodiment 1 or conjugate of embodiment 55 with CD81 under conditions suitable for blocking or reducing the interaction of a viral CD81 ligand or another CD81 ligand with CD81 or cells containing or expressing CD81.
112. The method of embodiment 101 that down-regulates T-cell receptor activity and/or that suppresses activity of natural killer cells, comprising contacting at least one small organic molecule according to embodiment 1 or conjugate of embodiment 55 with CD81 under conditions that block or reduce the interaction of a viral or another CD81 ligand with CD81 or cells containing CD81.
113. The method of embodiment 101 wherein the CD81 is on or expressed by a liver cell.
114. The method of embodiment 101, wherein the CD81 is on or expressed by a blood cell or on a precursor cell for a blood cell.
115. The method of embodiment 101, wherein the CD81 is on or expressed by a cell that is associated with HCV infection.
116. The method of embodiment 101, wherein the CD81 is on or expressed by a cell is associated with inflammation.
117. The method of embodiment 101, wherein the CD81 is on or expressed by a cell that is associated with down regulating T-cell receptors and suppressing the activity of natural killer cells.
118. The method of embodiment 101, wherein the CD81 is on or expressed by a cell that is associated with a HCV or non-HCV viral infection.
119. A method for modulating a biological activity of CD81 or an activity mediated by or through CD81 comprising contacting CD81 or a cell having CD81 with at least one molecule of embodiment 1 or a conjugate thereof
120. A method for inhibiting the attachment of a pathogen that binds to CD81 to a cell having CD81 comprising contacting said cell with at least one molecule of embodiment 1 or a conjugate thereof
121. The method of embodiment 120, wherein said pathogen is Hepatitis C Virus (HCV).

The invention is not limited to the embodiments described above; other aspects of the invention will be apparent from the disclosure below.

### BRIEF DESCRIPTION OF THE DRAWINGS

The patent or application file contains at least one drawing executed in color.

FIG. 1. Flowchart of the algorithm used by AutoLigand. The calculations are broken into three parts: Flood Fill, Optimization, and Ray Casting. The user enters the size (number of Fill points) to be used.

FIGS. 2A and 2B. Amino acid residues that participate in HCV E2 binding to CD81-LEL. Seven amino acids have been identified to contribute to the binding of the HCV protein E2 to CD81-LEL. (FIG. 2A) Front view of the protein showing the four contact residues Leu162, Ile182, Asn184, and Phe186 in color. (FIG. 2B) Back side of the CD81-LEL protein showing the other three contact residues Thr163, Glu188 and Asp196. This figure was prepared using AutoDock Tools version 1.5.6.

FIG. 3. Free energy data for ligand binding sites identified on the surface of CD81-LEL calculated by AutoLigand. This figure was generated by plotting the total energy per volume versus the volume of each fill made from different amounts of fill points. The different colors depict the different fills that start in the same location. The most efficient fills are those that have the lowest total energy per volume using the smallest volume. This figure was prepared using AutoDock Tools version 1.5.6.

FIGS. 4A and 4B. Two ligand binding sites identified by AutoLigand on the open conformation of CD81-LEL (PDB ID: 1G8Q). These two sites were selected as docking targets based on their proximity to the amino acid residues that contact E2 and the calculated low free energy (high affinity) for ligands that would bind in this site. (FIG. 4A) The small gray spheres define the ligand binding site, Site 1, calculated by AutoLigand to be the best binding site. Ligands binding to this site would interact directly with several key amino acids in the E2 binding site. These spheres correspond to the green circle fill points in FIG. 2A located between 500 and 600 Å³. (FIG. 4B) The black spheres identify a second binding site, Site 2, calculated by AutoLigand on the opposite side of the protein. Ligands binding to this site should also contribute to the disruption of E2 binding. The black spheres correspond to the black square fill points shown in FIG. 2A located between 550 and 650 Å³. This figure was prepared using AutoDock Tools version 1.5.6.

FIGS. 5A, 5B and 5C. Analysis of the main HCV E2 binding site in CD81-LEL using AutoLigand and comparing the results of AutoLigand to that of the virtual screening runs. AutoLigand fill points not only identify cavities on the surfaces of proteins, but they also predict the structural features of ligands that would bind with the best affinity and selectivity to the protein at these sites. (FIG. 5A) The fill points provided by AutoLigand define the rough shape of ligands that would fit best into the cavity. Specific fill points are also color coded to identify particular atoms in the ligand that would interact optimally with the protein's atoms or functional groups in the regions surrounding the ligand. (FIG. 5B) Ligand 1 is shown bound to CD81-LEL in the location and orientation calculated by AutoDock. (FIG. 5C) The superposition of fill points (small spheres) and atom types (large spheres) in Ligand 1 is high (75-80%) indicating that this ligand should bind well in this particular site. This figure was prepared using AutoDock Tools version 1.5.6.

FIG. 6. Small molecules calculated by AutoDock to bind to different sites on CD81-LEL. The molecules are listed according to the assessed quality of the ligand and its interaction with CD81-LEL using AutoDock's calculated free energy of binding and the DPI and SPR binding data. Criteria used to define the quality of the ligands are: Strong—makes more than 5 contacts with protein, calculated to be selective and not calculated to bind to multiple sites, not too hydrophobic in addition to having an in silico binding energy of >−5, DPI binding of >0.3 radians and SPR binding response of >30 Response Units (RU); moderate—makes 4-5 contacts with protein, hydrophobic interactions contribute to binding in addition to having an in silico binding energy of >−3, a DPI binding of >0.15 radians and SPR binding response of >10; and weak—makes 3-4 contacts with protein in addition to having an in silico binding energy of <−3, a DPI binding of <0.15 radians and SPR binding response of <10 RU.

FIG. 7. Binding of ligand 1 to CD81-LEL as a function of ligand concentration. This binding experiment was performed using a Biacore T200. Using the data, ligand 1 was estimated to have a Kd of ˜201 μM based on an affinity fit.

FIGS. 8A and 8B. An example of a bidentate ligand conjugate designed using two ligands, one calculated to bind to Site 1 and a second calculated to bind to Site 2 on the open conformation of CD81-LEL. (FIG. 8A) Location and orientation of ligand 1 and 4 binding calculated by AutoDock. (FIG. 8B) Structure of a prototype bidentate ligand conjugate. The linker was created using a single miniPEG and lysine and the resulting spacing between ligands was sufficient to allow the individual ligands to bind to the calculated sites when linked together.

FIG. 9 depicts CD81 ligand binding Site 1 and the surrounding amino acid residues Ser160, Thr163, Ala164, Thr167, Ile181, Leu185, Glu188, and Gln192. The amino acid residues surrounding the cavity are colored red and marked with the residue number in white. An example of a ligand bound in the site is colored dark blue.

FIG. 10 depicts CD81 ligand binding Site 2 and the surrounding amino acid residues Val136, Asp138, Leu165, Thr167, Lys171, Asn173, Ser177, Asn180 and Ile181. The amino acid residues surrounding the cavity are colored red and marked with the residue number in white. An example of one ligand bound in the site is colored dark blue.

FIG. 11 depicts CD81 ligand binding Site 3 and the surrounding amino acid residues Glu152, Ser177, Asn180, Phe186, Glu188, and His202. The amino acid residues surrounding the cavity are colored red and marked with the residue number in white. An example of one ligand bound in the site is colored dark blue.

FIG. 12 depicts CD81 ligand binding Site 4 and the surrounding amino acid residues Ser177, Lys148, Glu152, Leu170, Asn173, Asn180 and Lys301. The amino acid residues surrounding the cavity are colored red and marked with the residue number in white. An example of one ligand bound in the site is colored dark blue.

FIG. 13 depicts CD81 ligand binding Site 5 and the surrounding amino acid residues Asp117, Lys121, Glu125, Gln132, Ser160, Thr161, Gln192, and Asp195. The amino acid residues surrounding the cavity are colored red and marked with the residue number in white. An example of one ligand bound in the site is colored dark blue.

FIG. 14 describes a procedure for making a multivalent ligand for CD81 tagged with biotin.

FIG. 15 describes a procedure for making a multivalent ligand for CD81 tagged with biotin.

FIGS. 16A, 16B, 16C, 16D, 16E, 16F, 16G, 16H, 161, and 16J describe chemical structures of ligands for CD81.

FIGS. 17A, 17B, 17C, 17D, 17E, and 17F depict ligand conjugates. Some preferred conjugates are 25678-lys-lys-40614, 75866-lys-lys-144958, 25678-lys-lys-75846 and 117922-lys-lys-75866.

FIG. 18 depicts a three ligand conjugate. SMILES

C(═O)(NC(C(═O)NCCCCC(N)C(═O)NC(═O)Cl(CCCCCl)NC)CCCCNC(C(C2=CC ═CC═C2)=CC3=CC═CC═C3)=O)C4=C(C═NC5=C4C═CC═C5)C6=CC═C(C═C6C)C.

### DETAILED DESCRIPTION OF THE INVENTION

The invention encompasses ligands for newly identified ligand binding sites on CD81. These ligands can be small organic molecules, conjugates of two or more small organic molecules directly linked together or linked together by a linker or spacer, or hybrid molecules engineered to contain chemical moieties of two or more small organic molecules involved in binding to CD81.

The inventors used several different methods to identify and characterize the small organic molecule ligands that bind to different sites on CD81. Additional methods for characterizing these molecules, for example, by determining a binding affinity of a molecule to CD81 or to a cell expressing CD81 or an ability of a molecule to inhibit binding of other ligands to CD81 in vitro or in vivo are described herein. Compositions containing these molecules and methods for their administration to subjects in need of modulation of CD81 biological activity, such as subjects infected with HCV are also described.

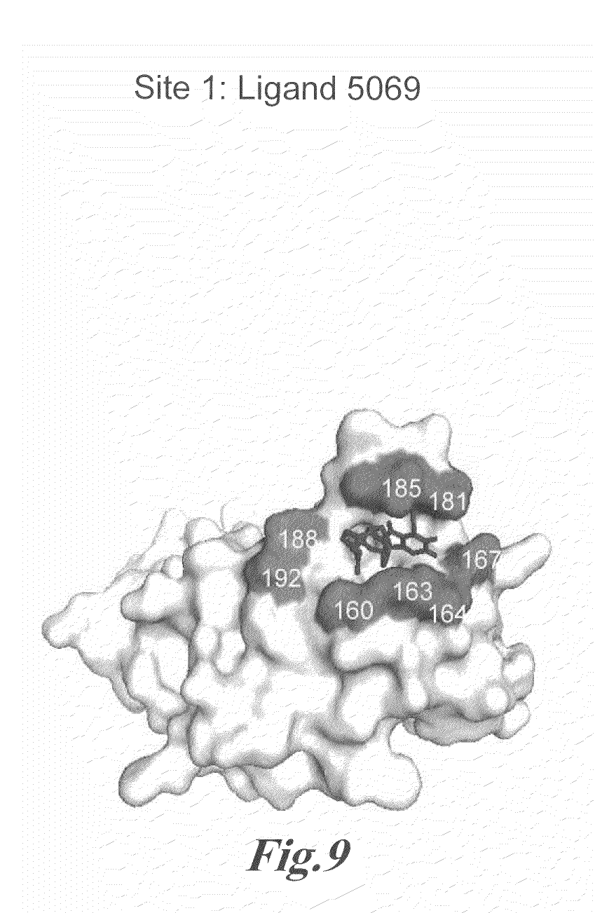

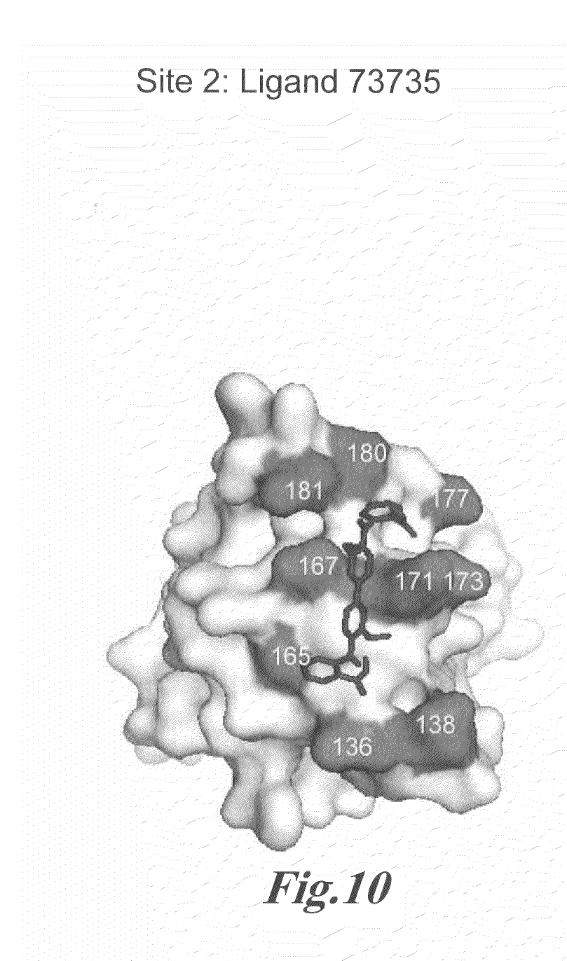

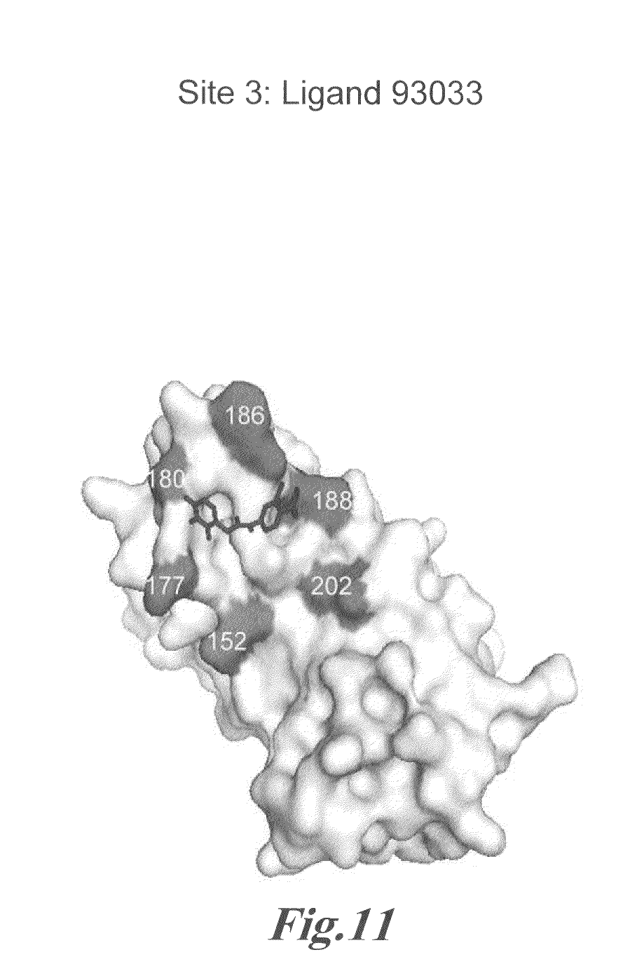

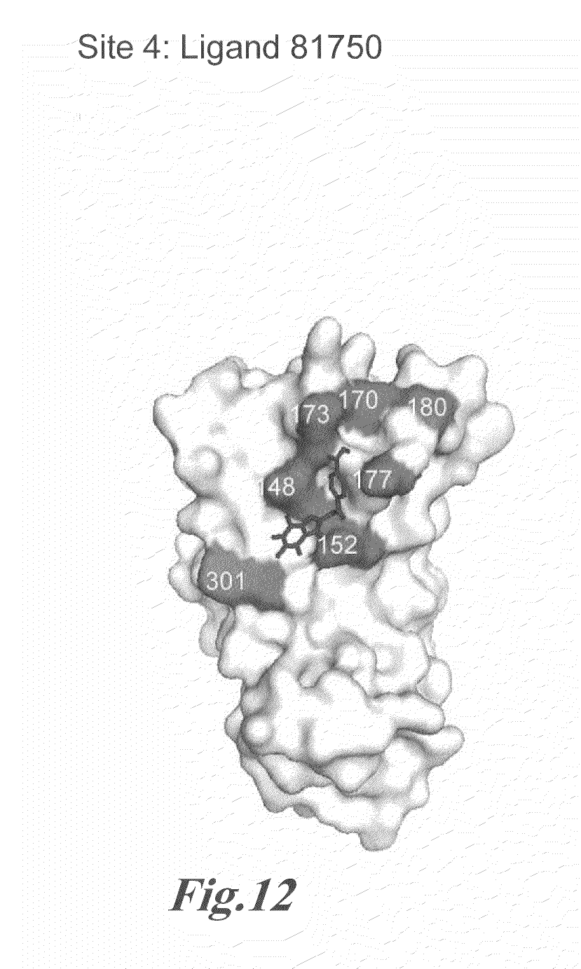

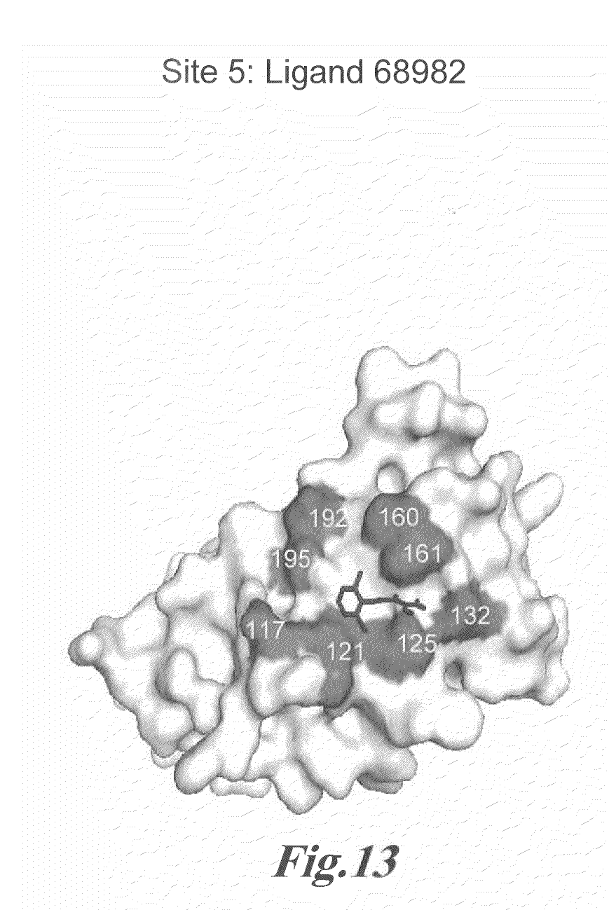

The inventors have identified ligands that bind to five different ligand binding sites on CD8181 as listed below. These ligands identify CD81 binding sites to which they bind and can be used to identify other molecules that recognize these sites, for example, CD81 by a competitive binding assay. The CD81 binding sites are described by the ligands identified below which bind to them, by the amino acids surrounding each binding site as shown by FIGS. 9-13, and by the binding sites depicted by FIGS. 9-13.

Table AA depicts ligands identified as binding to particular CD81 binding sites.

| Site 1 | Site 2 | Site 3 | Site 4 | Site 5 |
|---|---|---|---|---|
| 165665 | 38743 | 93033 | 16631 | 68982 |
| 164965 | 156957 | 80807 | 68971 | 75866 |
| 689002 | 127947 | 25368 | 78623 | 90444 |
| 30930 | 73735 | 16162 | 81750 | 148832 |
| 5069 | 55573 | 25678 | 401077 | 601359 |
| 7436 | 41066 | 60239 | 408734 | 142446 |
| 21034 | 11891 | 75866 | 303800 | |
| 98026 | 63865 | 87504 | 75846 | |
| 123115 | 408860 | 89720 | 638134 | |
| 7962 | 362639 | 215276 | 70980 | |
| 16646 | 36914 | 331931 | 90444 | |
| 106863 | 20586 | 20586 | 89720 | |
| 117922 | 23895 | 403374 | 25678 | |
| 120631 | 252359 | 8481 | 215276 | |
| 7962 | 403374 | 5856 | 16162 | |
| 117922 | | | 60239 | |
| 106863 | | | | |
| 23895 | | | | |
| 120631 | | | | |
| 16646 | | | | |
| 252359 | | | | |
| 134137 | | | | |
| 97538 | | | | |
| 94914 | | | | |
| 31712 | | | | |
| 73170 | | | | |
| 144958 | | | | |
| 153172 | | | | |

The terms used to describe the ligands and ligand binding sites are described below.

The term “CD81” is given its ordinary meaning in the art (Cluster of Differentiation-81). Human CD81 has been sequenced and is crystal structure determined. CD81 analogs from non-human animals are known and natural or artificial variants of CD81 are also contemplated. These are characterized by a degree of similarity or sequence identity to human CD81, for example, by a degree of similarity or identity of 80%, 85%, 87.5%, 90%, 92.5%, 95%, 97.5%, 98%, 99% to a known CD81 sequence, such as that described by SEQ ID NOS 2 and 3.

Similarly, the term “HCV E2” is given its customary meaning. The invention contemplates variants of the HCV E2 protein from different strains of HCV; analogs of this protein from other viruses or microorganisms, especially analogs of segments of the protein that interact with CD81, or other natural or engineered forms of the HCV E2 protein or its variants or analogs. These variants, analogs or forms of the E2 protein can be characterized by a degree of similarity or identity of 80%, 85%, 87.5%, 90%, 92.5%, 95%, 97.5%, 98%, 99% to a known HCV E2 sequence, such as that described by SEQ ID NO: 1. Related or similar viruses may be identified based on their expression of a protein having these degrees of similarity or identity to human HCV E2 protein.

BLASTP may be used to identify an amino acid sequence having at least 80%, 85%, 87.5%, 90%, 92.5%, 95%, 97.5%, 98%, 99% sequence similarity or identity to a reference amino acid sequence using a similarity matrix such as BLOSUM45, BLOSUM62 or BLOSUM80. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure.

A “small organic molecule” includes low molecular weight organic compounds or approximately 800 daltons or less that are not polymers. Small molecules according to the invention will bind to CD81. These small molecules may bind to a particular site on CD81, such as Site 1, 2, 3, 4, or 5 discovered by the inventors, or to more than one site. These ligands may have a greater or lesser affinity for CD81 than a natural ligand from CD81, such as the HCV E2 protein. Ligand binding can passively block binding of other ligands to CD81 and/or trigger or transduce a signal by binding to CD81 thus modifying the function or activity of CD81, for example, by inhibiting binding of HCV E2 ligand to CD81 and thus inhibiting binding of HCV to CD81-bearing cells.

The binding affinity and ligand efficacy of a CD81 ligand molecule can be determined by methods known in the art. Different ligands will exhibit different binding affinities for CD81, for example, binding affinity can range from 1 nM to 10,000 nM and all intermediate values within this range, such as 1 nM, 10 nM, 100 nM, 1,000 nM, 5,000 nM and 10,000 nM. The inventors have found that ligands that bind to at least two of Sites 1, 2, 3, 4, and 5 identified on CD81, bind more strongly to CD81 than individual ligands for each site.

The invention contemplates such small molecules per se, as well as larger conjugates or hybrid molecules containing one or more small molecules that interact with CD81. The larger conjugates or hybrid molecules may comprise more than one determinant that binds to CD81, more than one copy of a particular CD81-binding determinant, or determinants that bind to different sites on CD81.

Small organic molecules according to the invention are publicly available, for example, as described in the ZINC database. ZINC is a free database of commercially-available compounds for virtual screening. ZINC contains over 21 million purchasable compounds in ready-to-dock, 3D formats. ZINC is provided by the Shoichet Laboratory in the Department of Pharmaceutical Chemistry at the University of California, San Francisco (UCSF), see: Irwin, Sterling, Mysinger, Bolstad and Coleman, J. Chem. Inf. Model. 2012DOI: 10.1021/ci3001277. The original publication is Irwin and Shoichet, J. Chem. Inf. Model. 2005; 45(1):177-82PDF, DOI. The compounds described in the ZINC database as of Sep. 22, 2012 are incorporated by reference to the Zinc database or to the publications above.

Functional variants of the small organic molecules of the invention are also contemplated. Like the unmodified small organic molecule, these variants will bind to CD81 but may have one or more substitutions to the chemical structure of the unmodified small organic molecule ligand. Other substitutions to the core structure of a small organic molecule ligand described herein include other functional groups that improve i) binding to CD81, ii) confer specific properties such as those related to solubility, stability, pharmacokinetics, biodistribution, absorption, tissue uptake, residence time in tissue, or ones that minimize toxicity, excretion or metabolism, iii) enable the small molecule ligand to be conjugated to other molecules, or iv) facilitate the diagnostic use of the small molecule ligand.

Examples include the addition or substitution of other atoms such as halogens (chlorine, fluorine, iodine, bromine), metals or radioisotopes (to enable detection or visualization), tags such as fluorescent dyes or molecules, biotin, digoxigenin, peptides amino acids (to improve uptake, delivery and biodistribution), or functional groups such as carboxylic, amino, amine, amide, azo, ester, thiol, sulfonyl, nitro, alkoxy, acetyl, acetoxy, hydroxyl or other alcohol, aldehyde, carbonyl, alkyl, alkene or alkyne groups or chains, ether, epoxide, hydrazone, imide, imine, isocyanate, isonitrile, isothiocyanate, ketone, nitrile, nitrene, nitro, nitroso, organophosphorus, oxime, phosphonic or phosphonous acid, sulfone, sulfonic acid, sulfoxide, thiocyanate, thioester, thioether, thioketone, urea, pyridine groups or other aromatic rings.

In some embodiments of the invention linkers or spacers are used. These linkers or spacers may be used to join small molecules that bind to different portions of CD81 and to space the small molecule moieties in a joined molecule so that they can bind to different parts of CD81. For example, a small molecule that binds to Site 1 on CD81 may be spaced from 0 (e.g., where a carboxyl group on one small molecule ligand is coupled to an amine group on another) to about 30 Å (3 nm) apart from one that binds to Site 2 using a linker of an appropriate length. In most cases, linkers would range from 2 or 3 to about 7-10 Å. Generally, small organic ligand molecules will be joined by linkage to a single position on each ligand to another ligand or to an intervening linker. However, linkage may also occur at 2 or more positions on a ligand molecule to another ligand molecule or linker. Linkers may have different chemical structures including straight-chain and branched chain structures, and structures including saturated or unsaturated bonds (e.g., alkyl, alkenyl or alkynyl), heteroatoms (e.g., nitrogen, oxygen or sulfur) or aromatic moieties. Bivalent and multivalent linkers may contain the same or different reactive chemical groups for linking two or more small molecule ligands for CD81. Linkers may range from 1, 2, 3, 4, 5, 6, 7, 8, 9, 10, 11, 12, 13, 14, 15 or more atoms in length. Direct linkages between two or more small molecule ligands may also be used to form conjugates of CD81 ligands where each ligand has a chemical group that can react with a chemical group on another ligand.

Linkers suitable for use in the invention are known in the art and are incorporated by reference to Ducry, et al., Bioconjugate Chem. 21, 5-13, Antibody-Drug Conjugates: Linking Cytotoxic Payloads to Monoclonal Antibodies (2010); to Gordon, et al., J. Chem. Technol. Biotechnol. 74:835-851, Solid phase synthesis—designer linkers for combinatorial chemistry: a review (1999), and to Leitner, et al., Mol. Cell. Proteonom. 9:1634-1649 (2010), which are incorporated by reference. Exemplary linkers include lysine and polyethylene glycol (PEG) moieties.

Generally, the small molecule ligands of the invention are not polymers. However, conjugates of small molecule ligands may contain multiple units of one or more small organic molecule ligands, for example, as linked to a dendrimer. In addition to small organic molecules linked together with a chemical linker, these small organic molecule ligands may be conjugated to larger moieties such as antibodies and other proteins, nucleic acids and nucleic acid analogs, carbohydrate and sugar molecules, etc. The small molecule ligands, conjugates or hybrids may also be conjugated to detectable moieties such as avidin or streptavidin, biotin or other detectable tags.

Hybrid molecules that comprise chemical moieties from two or more known small organic molecule ligands are engineered by a process of fragment-based extension.

A “composition” or “pharmaceutical or therapeutic composition” according to the invention refers to a combination of carrier, excipient, or solution with a small molecule, ligand conjugate or hybrid molecule. The term “pharmaceutically acceptable carrier” includes any and all carriers and excipients such as diluents, solvents, dispersing agents, emulsions, lipid bilayers, liposomes, coatings, preservatives including antibacterial or antifungal agents, isotonic agents, pH buffers, and absorption modulating agents, and the like, compatible with the molecules of the present invention and suitable for pharmaceutical administration. The use of such carriers, disintegrants, excipients and agents for administration of pharmaceutically active substances is well known in the art, see the Handbook of Pharmaceutical Excipients, 3^{rd} edition, Am. Pharm. Assoc. (2000) which is incorporated by reference. The pharmaceutical compositions of the invention are generally formulated for compatibility with an intended route of administration, such as for parenteral, oral, or topical administration.

The therapeutic compositions of the invention include at least one molecule according to the invention in a pharmaceutically acceptable carrier. A “pharmaceutically acceptable carrier” will be at least one component conventionally admixed with, and used for, the administration of an active ingredient, biological product, or drug. A therapeutic composition may be sterile or in a form suitable for administration to a human or non-human subject. A carrier may contain any pharmaceutical excipient used in the art and any form of vehicle for administration. The compositions may be, for example, injectable solutions, aqueous suspensions or solutions, non-aqueous suspensions or solutions, sprays, solid and liquid oral formulations, salves, gels, ointments, intradermal patches, creams, lotions, tablets, capsules, sustained release formulations, and the like. Additional excipients may include, for example, colorants, taste-masking agents, solubility aids, suspension agents, compressing agents, enteric coatings, sustained release aids, and the like. A suitable dosage form may be selected by one of skill in the art from forms such as those described by “Dosage Form”; NCI Thesaurus OID: 2.16.840.1.113883.3.26.1.1 NCI concept code for pharmaceutical dosage form: C42636; accessible at http://www.fda.gov/ForIndustry/DataStandards/StructuredProductLabeling/ucm162038.htm (last accessed Sep. 20, 2012) which is hereby incorporated by reference.

Orally administered compositions include a solid carrier or excipient or may be formulated as liquid or gel preparations and may include an edible or inert carrier and may be enclosed in capsules, compressed into tablets, or formulated as a troche. Orally administered compositions may be prepared in a time-release or encapsulated form to prevent degradation in the stomach and optimize uptake of a molecule.

Injectable compositions may be formulated by methods well known in the art and may encompass sterile solutions or dispersions of therapeutic molecules. Such will usually include a sterile diluent, such as water, normal saline, or other buffer compatible with the molecules of the invention. Injectable compositions may be prepared in unit dosages or in unit dose containers, such as vials, ampules, or syringes.

Conventional buffers and isotonic agents may be used and pH may be adjusted using well known agents, such as HCl or NaOH or buffers. Antimicrobial or bacteriostatic agents, chelating agents, such as EDTA or EGTA, and antioxidants and preservatives may be present.

The therapeutic compositions of the invention may be administered by any acceptable route of administration including topically, on to a mucous membrane, orally or enterically or parenterally. These routes include, but not limited to topical, transmucosal, orally (including buccal, sublingual), mucosally (conjunctiva, nasal, sinal, urethral, vaginal, intestinal, rectal), enteric, transdermal, intradermal, subcutaneous (s.c.), intramuscular, intraperitoneal, intravenous (i.v.) intracardiac, into a joint or bone, into an organ (brain, spinal chord, eye, ear, liver, spleen, kidney, gall bladder, bladder), into bone, cartilage, or joint tissue, by inhalation (e.g., intranasal, intratracheal, intrapulmonary, or intrabroncial), oral, subuccal. Routes may be selected by those of skill in the art from those listed in the U.S. FDA, CDER, Data Standards Manual “Routes of Administration”; FDA Data Element Number. None. CDER Data Element Number. C-DRG-00301; Data Element Name. Route of Administration; Data Element OID: 2.16.840.1.113883.3.26.1.1.1 Data Element NCI Concept ID: C38114; Version Number 004 accessible at http://_www.fda.gov/Drugs/DevelopmentApprovalProcess/FormsSubmissionRequirements/ElectronicSubmissions/DataStandardsManualmonographs/ucm071667.htm; (last accessed Sep. 21, 2012) which is hereby incorporated by reference.

### Example 1

### Preparation of CD81-LEL Structure and Calculation of Binding Sites

The AutoDock suite of programs, developed by Dr. Arthur Olson's molecular graphics laboratory at the Scripps Research Institute, was used to analyze the large extracellular domain of our target protein CD81, prepare surface grid maps, and dock a library of small molecules into several cavities located in the vicinity of amino acid residues known to participate in E2 binding. The AutoDock scoring function employs a subset of the AMBER force field, implementing the united-atom model [25]. AutoGrid, a second program included in the AutoDock suite, pre-calculates these grids. AutoDock Tools (ADT) is the graphical user interface [25-28] that helps visualize the grid box and determine the desired site, in addition to analyzing the docking results. AutoLigand [29], a recently developed AutoDock tool, was designed to identify potential binding sites in known protein structures. This tool works by finding a set of fill points that have the strongest possible interaction energy with the protein. These fill points are then used to identify the best small molecule virtual screening hits [29].

The coordinates for the crystal structure of the open conformation of CD81-LEL (PDB ID: 1G8Q) were obtained from the Protein Data Bank (PDB). AutoDock Tools (ADT) 1.5.6 [25-28] was used to delete water molecules, add polar hydrogens, assign Gasteiger charges, and create grid bounding boxes with a 1 Å spacing for use with AutoLigand and a 0.375 Å spacing for use with AutoDock 4.2. AutoGrid 4.2 was used to pre-calculate grid maps of interaction energies for various atom types and create the map files that were used by AutoLigand to find the CD81-LEL binding sites and by AutoDock for docking. The affinity at each grid point was calculated in AutoGrid by using pair-wise energetic terms with all surrounding atoms which include evaluations for dispersion/repulsion, hydrogen bonding, electrostatics, and desolvation:

$$V = {W}_{vdw} {\sum }_{i , j} ( \frac{{A}_{ij}}{{r}_{ij}^{12}} - \frac{{B}_{ij}}{{r}_{ij}^{6}} ) + {W}_{hbond} {\sum }_{i , j} E ( t ) ( \frac{{C}_{ij}}{{r}_{ij}^{12}} - \frac{{D}_{ij}}{{r}_{ij}^{10}} ) + {W}_{elec} {\sum }_{i , j} \frac{{q}_{i} {q}_{j}}{ɛ ( {r}_{ij} ) {r}_{ij}} + {W}_{sol} {\sum }_{i , j} ( {S}_{i} {V}_{j} + {S}_{J} {V}_{i} ) {e}^{( - {r}_{ij}^{2} / 2 {σ}^{2} )}$$

The weighting constants W have been optimized to calibrate the empirical free energy based on a set of experimentally determined binding constants [28, 29]. The first term, W_{vdw}, is a typical 6/12 Lennard-Jones potential for dispersion/repulsion interactions. The parameters are based on the AMBER force field. The second term, W_{hbond}, is a directional H-bond term based on a 10/12 Lennard-Jones potential. The parameters C and D are assigned to give a maximal well depth of 5 kcal/mol at 1.9 Å for hydrogen bonds with oxygen and nitrogen, and a well depth of 1 kcal/mol at 2.5 Å for hydrogen bonds with sulfur. The function E(t) provides directionality based on the angle t from ideal hydrogen-bonding geometry. The third term, W_{elec}, is a screened Coulomb potential for electrostatics. Wₛₒₗis a desolvation potential based on the volume of atoms (V) that surround a given atom and shelter it from solvent, weighted by a solvation parameter (S) and an exponential term with distance-weighting factor σ□=3.5 Å. A and B are constants that describe the magnitude of the repulsive and attractive terms i and j are the iteration numbers of the atoms being examined. So when i=3 and j=147, all of the forces between atom 3 and atom 147 are being calculated. r is the distance between atom i and j, q is the charge and epsilon is the dielectric constant. σ is the distance weighting factor that is set to: s=3.5 Å [25-27].

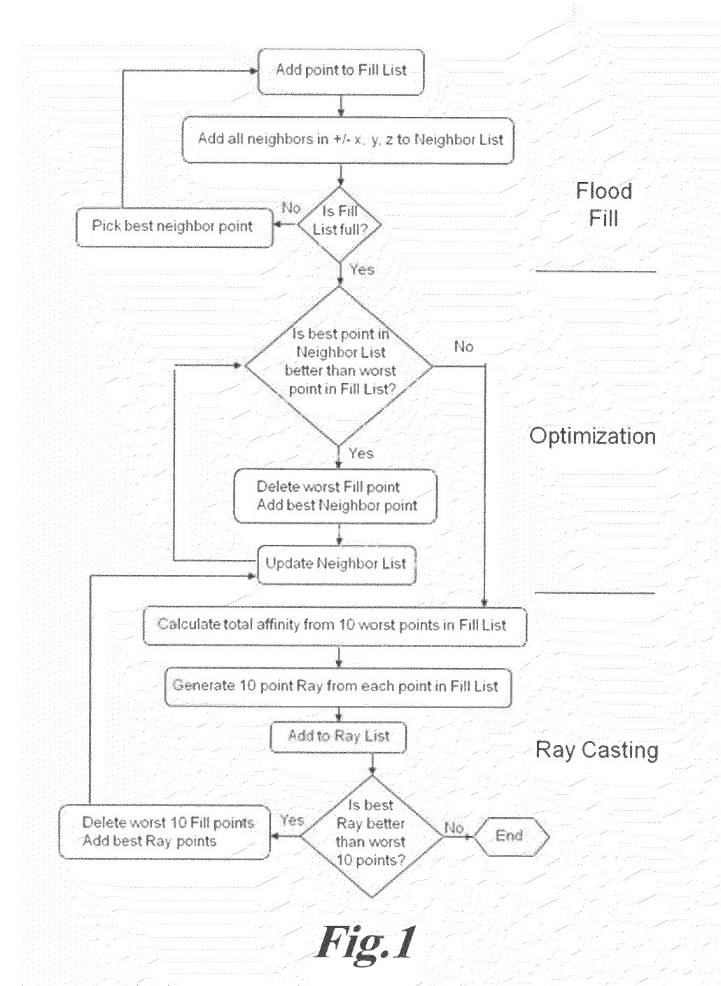

AutoLigand was used to rapidly scan the protein for high affinity binding pockets and identify the optimal volume, shape, and best atom types for each binding site. This was accomplished by filling each cavity on the surface of the protein with a contiguous set of affinity points using three steps: a) flood fill, b) local migration, and c) ray casting [29]. These fill points are a grid-based representation of carbon, oxygen, and hydrogen atom centers that correspond to atomic centers of ligand atoms that fill a site, interact with the atoms in the protein and maximize the ligand's affinity within the site. By varying the number of fill points, the program determines the best total affinity per volume of the fill as the best binding site. The flowchart in FIG. 1 shows the algorithm used by AutoLigand. An overall loop is used to scan through each point in the grid box as the seed point for the algorithm. After each fill is generated, only the top ten non-duplicated fills are reported since many fills will overlap and produce the same end solution.

The CD81-LEL protein was scanned by AutoLigand using fill sizes from 10 to 210 fill points. The constructed grid box enclosed the entire protein with dimensions of 40 Å by 18 Å by 38 Å and was centered on 3.144, 34.966, and 15.812 in the protein frame of reference. Five potential ligand binding sites were identified on the open CD81-LEL structure (PDB code 1G8Q). Two sites located adjacent to amino acid residues critical for E2 binding were selected for docking.

### Virtual Screening

AutoDock 4.2 [25-28] was used to perform virtual screening runs using a subset of the ZINC small molecule database containing 10,000 molecules taken from the National Cancer Institute-Diversity Set II (NCI_DSII), Sigma, and Asinex libraries. The parameters were set at 100 for the number of genetic algorithm (GA) runs, 150 as the population size, and a maximum number of generations of 25,000. The Lamarckian genetic algorithm in AutoDock was used to perform the docking experiments [30]. Docking results were sorted by the lowest binding energy in addition to specific ligand selection criteria that would facilitate the design and synthesis of the best ligand conjugates. The virtual screening runs were performed using the National Biomedical Computation Resources (NBCR) computer cluster [31]. Vision [32] was used to construct the computational workflows that were used for virtual screening on the NBCR cluster. The list of small molecules calculated to bind to the two sites were ranked according to their calculated free energy of binding, and those with the lowest free energies were further screened manually to identify the best ligand candidates for experimental testing.

### Ligand Evaluation

Several criteria were considered in the selection of the ligands used to design the ligand conjugates. During the initial examination of the list of ligands calculated to bind to each site by AutoDock, only ligands containing one free carboxyl group or one amino group (or one of each) were selected. In the most highly ranked cases, these amino or carboxyl groups were not buried in a cavity nor did they interact with the protein surface. They were exposed to solvent and were calculated by AutoDock to bind to the protein with the functional group pointed in the general direction of the second ligand binding site. Preference was given to ligands that were calculated to form multiple contacts with atoms or amino acid residues in or around the perimeter of the targeted cavities. Ligands containing two or more free amino or carboxyl groups were only considered for use in creating ligand conjugates that might need additional charge to increase their solubility. Molecules that were highly hydrophobic, highly charged, known to be toxic, exist in more than one form (such as enol-keto forms), or contained disulfide bonds were avoided. After manually filtering the ligand sets to remove the molecules that did not meet these criteria, the calculated binding energy was used to identify the top hits.

### Surface Plasmon Resonance

SPR analysis was performed using a Biacore T200 workstation (GE Healthcare, NJ, USA). A recombinant form of the CD81-LEL protein with a GST tag (generously provided by Dr. Shoshana Levy, Stanford University) was used to confirm, using an established experimental technique, the binding of several ligands to the protein. Briefly, 10 μM CD81-LEL-GST diluted into 10 mM Na-Acetate buffer pH 4.5 was immobilized for 15 min at a flow speed of 5 μl/min onto a CM5 sensor chip using amine-coupling (EDC-NHS). Approximately 20,000 RU of protein were immobilized on the chip. The ligands were prepared as 600 μM solutions in PBS-0.05% Tween-80 (the running buffer) and they were introduced to the protein using a pre-programmed 3 minute association and 1 minute dissociation interval.

The binding affinity of the most tightly bound ligand in the group selected for ligand conjugate design (Ligand 1) was estimated using data collected from a series of SPR binding experiments conducted at different ligand concentrations. To obtain the kinetic and affinity data needed to estimate the Kd, the original ligand was diluted serially with running buffer to produce seven different ligand concentrations: 1024 μM, 516 μM, 256 μM, 128 μM, 64 μM, 32 μM and 0 μM. Data were fitted using a monovalent binding model.

### Dual Polarization Interferometry Analysis

DPI analyses were performed using an AnaLight 4D workstation (Farfield Group, Manchester UK). The recombinant CD81-LEL was immobilized onto a Thiol AnaChip using Sulfo-GMBS as a cross-linker in PBS running buffer. Non-specific sites were blocked with digested casein. TRIS was used to cap the cross-linker, blocking any additional amines from covalently binding to the cross-linker on the chip surface. Ligands were prepared as a 20 mM stock solutions in DMSO. Each ligand was diluted to a final concentration of 500 μM in PBS just prior to injection (final DMSO concentration was 2.5%). PBS and DMSO mixed in the same ratio were used as a blank. Data collection and analysis were performed using the AnaLight Resolver.

### In Silico Design of Conjugated Ligands

The pair of ligands (one from Site 1 and one from Site 2) used to design the prototype ligand conjugate were selected using three criteria: 1) their calculated free energy of binding to CD81-LEL, 2) the strength of binding to the protein as determined by SPR, and 3) a confirmation of the ligand's binding to CD81-LEL using DPI. Using the structure of the bound ligand-CD81-LEL complexes calculated by AutoDock, the distance between the carboxyl groups on Ligands 1 and 4 was estimated and a linker of suitable length was designed using a combination of lysine and miniPEG molecules. The linker spacing was optimized by incorporating PEG moieties between the ligands to adjust the length of the linker and by inserting lysine residues at key points to enable linker branching [23, 24].

### Target Regions on CD81-LEL

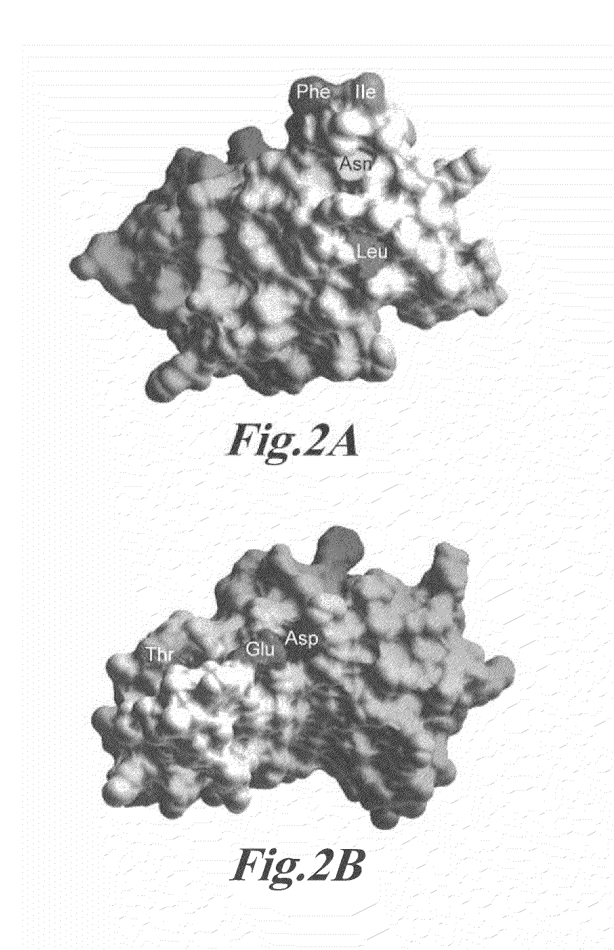

The crystal structure of the open CD81-LEL conformation was used as the target for the virtual screening runs performed using AutoDock to identify small molecule ligands calculated to bind to cavities that encompass or are located near known E2 contact residues. Based on mutation studies, Higginbottom et al. [17] identified four residues that were considered to be essential for the HCV E2 protein to bind to CD81-LEL. The D196E mutation in CD81 was observed to reduce binding to E2. In addition mutations F186L and E188K inhibited binding of CD81 to E2, whereas T163A enhanced their interaction [17]. Drummer et al. [19] also examined the binding site, which was estimated to cover approximately 806 Å² of the CD81-LEL surface, and identified three additional amino acid contacts, Ile182, Asn184, and Leu162 [19] (FIG. 2). We used these seven residues as markers to identify the best regions on the CD81-LEL protein surface to target when designing inhibitors to block the E2:CD81 interaction.

### The AutoLigand Fill Points and Energy Plot Analysis

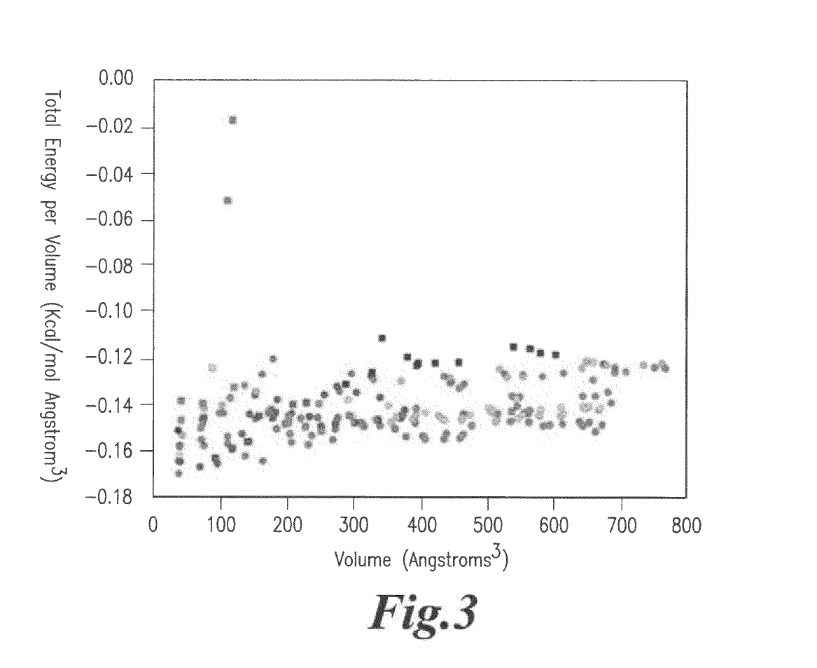

AutoLigand was used to analyze the surface of CD81-LEL and select the best ligand binding sites. Five binding sites were identified as potential targets by plotting the total energy per volume (Kcal/mol Å³) for the fill points generated against the volume of the filled site and picking those sites with the lowest values. FIG. 3 shows the data from each fill generated at different starting points on the surface using increasing numbers of fill points to fill larger and larger volumes. The fill volumes with less than 100 Å³ are small cavities within the protein structure that could be water or ion binding sites and were not considered suitable drug targets. The dark green circles plot the values for the fills near amino acid Asn184, one of the five key residues shown previously to interact with E2. The Best fill for the site in this region, −0.165 Kcal/mol Å³, was obtained using 180 fill points. As more points were used and the volume of the cavity increased, the calculated free energy of binding became less favourable.

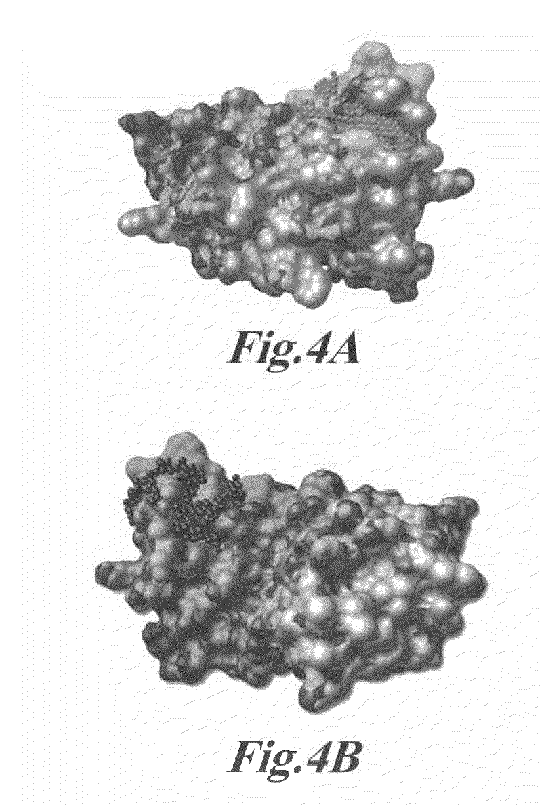

One site calculated by AutoLigand to be an excellent small molecule binding site was located in a region that contained five of the CD81 amino acid residues (Ile182, Phe186, Asn184, Glu188, Asp196) [19] that have been shown by others to interact with E2 (FIG. 4A). A second group of fill points was generated for a neighboring cavity located on the opposite side of the protein (FIG. 4B). The fill points generated for these two sites were calculated to have the lowest interaction energy of all the sites identified on the open conformation of CD81-LEL. Consequently, these two sites were selected as the primary sites for use in small molecule docking and ligand conjugate development. Additional regions determined to be good ligand binding sites were used to identify other small molecules that could be linked to ligands calculated to bind to the first two sites to enable the creation of a future tridentate ligand conjugate.

### Docking and Analysis of Ligands Calculated to Bind to the Selected Sites

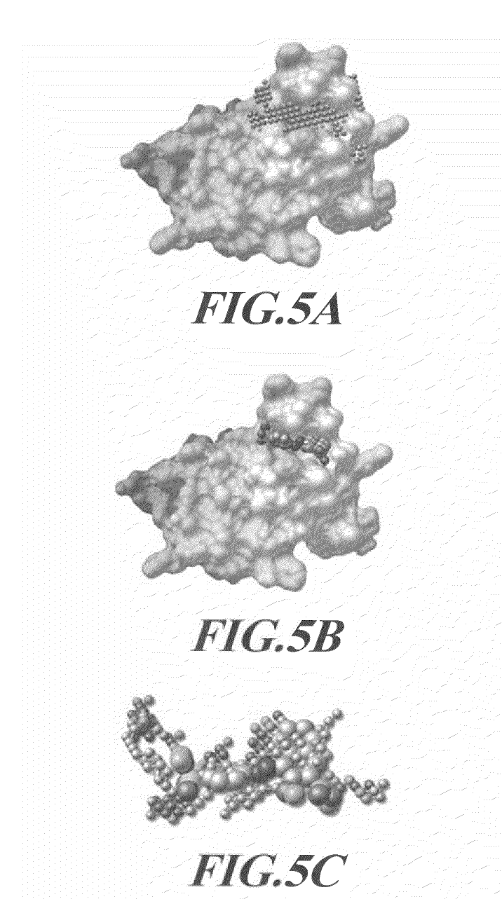

Docking runs were performed for the sites selected on CD81-LEL using the NCI Diversity Set II of small molecules. The list of ligands calculated to bind to each site were ranked according to binding energy and how well the ligand's atoms mapped onto the fill points for the site. In addition to the fill points defining the rough shape of ligands that would fit best within the cavity, specific fill points were also color coded to identify particular atoms (carbon, hydrogen, nitrogen or oxygen) in the ligand that would interact optimally with the surface of the protein in the regions surrounding the ligand (FIG. 5). The fill points calculated for the site shown in FIG. 5A are colored red (for hydrogen acceptors such as oxygen or nitrogen), blue for hydrogens, or gray for carbons. One of the better ligands calculated to bind to this site (FIG. 5B) has atoms that superimpose well with the fill point map (FIG. 5C). While the superimposition does not need to match perfectly, the points of contact on the protein are considered to be good if the majority of the different atom types in the molecule (75-80%) approximates the same location as the fill points.

The highest ranking ligands were further evaluated based on several additional criteria. The most important is the requirement that selected ligands contain a single amino or carboxyl group. Ligands that contain either group can easily be conjugated to a linker (miniPEG or a lysine) using the simple peptide-based chemistry used to rapidly synthesize ligand conjugates [23]. The second requirement involved the orientation of the bound ligand. Ligands were only selected if the free carboxyl or amino group were calculated to point out toward solvent and in the general direction of the second binding site. This would maximize the probability that the ligand could still bind to the protein when conjugated to the linker, and it would enable both ligands to bind to their respective cavities after being linked together. In addition, those ligands that formed multiple contacts/interactions with the protein (such as hydrogen bonds, salt bridges, van der Waals interactions) were considered to be better than those calculated to make only one or two contacts.

### Experimental Confirmation of Ligand Binding

A total of 36 ligands were tested experimentally using surface Plasmon resonance (on a Biacore T200 instrument) to identify which of the molecules calculated to bind to Sites 1 and 2 on CD81 actually bind to a recombinant form of the protein (CD81-LEL). Twenty-six of the molecules provided a positive change in response units (RU) upon introduction to a chip containing the immobilized protein (Table A), indicating the ligands bound to the protein. The measured responses for the ligands that bound varied from 2.3 to 78.4 RU. Those ligands providing the largest responses tended to be molecules that were calculated to bind more deeply inside cavities in Site 1 (ligands 30930, 98026, 7436, 5069), Site 2 (ligands 127947, 38743) or Site 4 (ligands 78623, 16631).

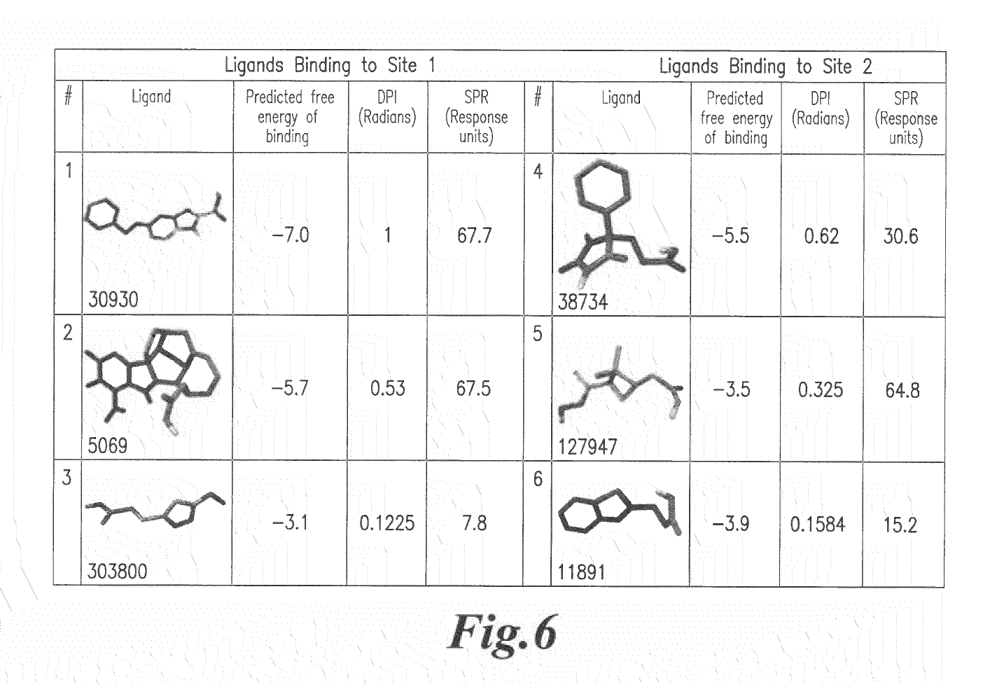

Six of the more interesting ligand candidates being considered for use in creating a ligand conjugate (three calculated to bind to Site 1 and three calculated to bind to Site 2) were further tested to confirm binding using a second experimental technique, dual polarization interferometry. The results, shown in FIG. 6, confirmed that all six ligands bound to the protein. The relative rank in strength of binding of the Site 1 and 2 ligands, as determined by DPI, were also similar to the ranking obtained by SPR or calculated by AutoDock for the majority of the ligands. Ligands 1 and 4 exhibited binding responses that were stronger than that observed for benzyl salicylate (0.58 radians), a small molecule reported previously to block E2 binding to CD81 [33]. In some cases, significant differences were observed in the actual binding responses obtained by SPR and DPI. As one example, Ligands 1, 2 and 5 had very similar binding data when tested by SPR, but these ligands exhibited quite different values when tested by DPI. One reason for this observed difference in the DPI response may relate to conformational changes in the protein that occur when the small molecules bind. The change in radians measured using DPI when a ligand binds to a protein is known to result from a combination of two effects: 1) the resulting increase in mass/volume when the ligand binds to the protein on the surface of the chip and 2) a conformational change in the protein induced by the binding of the ligand. Molecules binding in the deeper cavity would be expected to have more and stronger contacts with the protein than ligands sitting exposed to solvent in shallow cavities or surface binding sites.

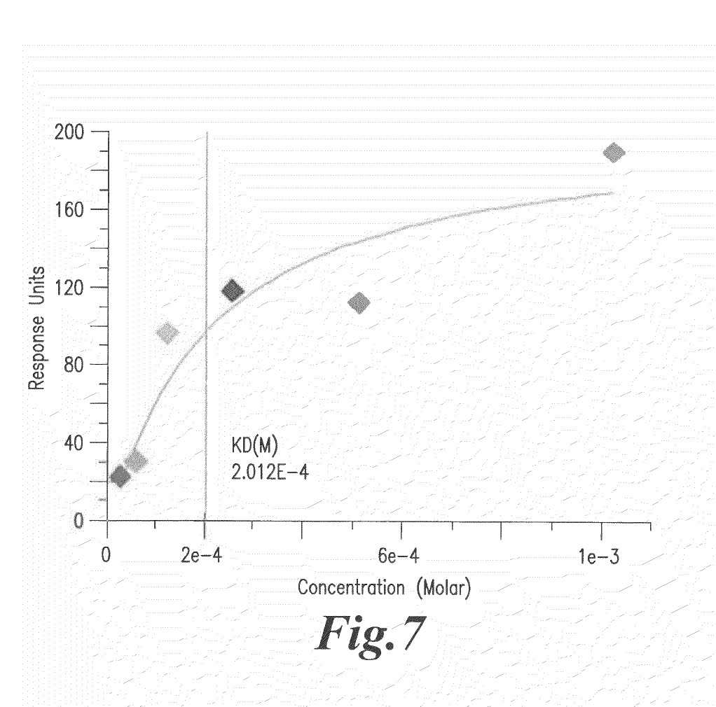

Those molecules calculated by AutoDock to have the lowest free energy of binding usually also exhibited the largest DPI radian change and SPR response. The collective data provided by the AutoDock free energy calculation, SPR, and DPI binding assays allowed us to estimate and categorize the relative strength of the ligand's binding to CD81-LEL as strong, moderate or weak. Within the set of six ligands shown in FIG. 6, Ligands 1, 2 and 4 exhibit the strongest binding, followed by ligands 5 and 6, which are categorized as moderate binders. Ligand 3 appears to be the weakest binder in the group. Additional SPR analyses performed using a series of Ligand 1 concentrations (FIG. 7) provided an estimated Kd of 201 μM for an affinity fit of Ligand 1 binding to the recombinant CD81-LEL.

### In Silico Design of Ligand Conjugates

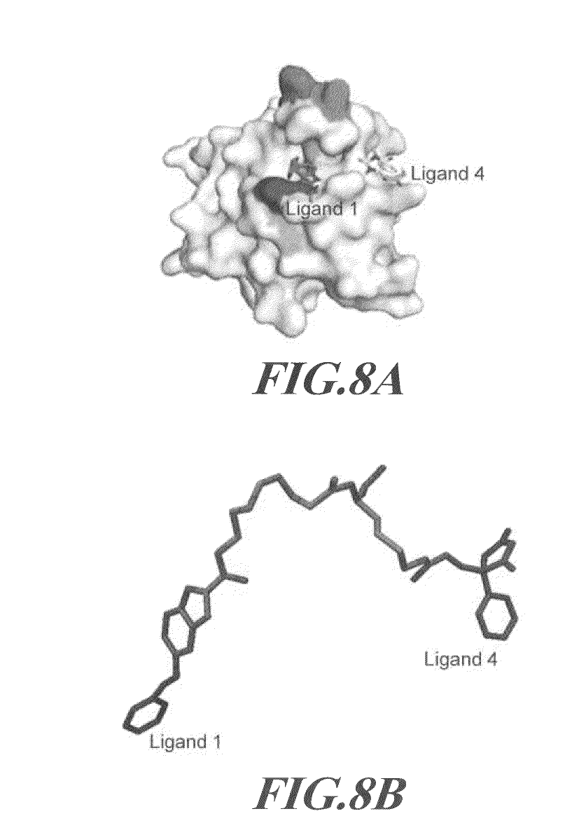

Based on these DPI and SPR binding results and the site of binding and orientation of the bounds ligands calculated by AutoDock, Ligands 1 and 4 were used to design the first anti-HCV ligand conjugate (FIG. 8B). This bidentate ligand conjugate, which was designed to bind two different sites on the open conformation of CD81-LEL located within the E2 binding site. To create the HCV ligand conjugate, the carboxyl group of one ligand was conjugated to the alpha amino group of a lysine. The amino group of a functionalized miniPEG was linked to the carboxyl group of the same lysine, and the amino group of another ligand was conjugated to the carboxyl group at the other end of the miniPEG using a type of chemistry (carbodiimide-based) that would produce amide groups at each conjugation point. The distance between two ligands bound to the surface of CD81-LEL was used to determine that only a single miniPEG molecule would be required to link the two ligands together with sufficient separation to allow both ligands to bind to their respective sites simultaneously. FIG. 8 shows the structure of the prototype bidentate ligand conjugate constructed using ligand 1 and ligand 4.

Table A.

Experimental analysis of ligand binding to recombinant CD81 LEL. Forty ligands calculated by AutoDock to bind to sites 1 and 2 on CD81-LEL were tested experimentally using surface Plasmon resonance as described in the Materials and Methods section. Ligand code numbers are those assigned by the National Cancer Institute. The data, which were obtained by two different methods (surface Plasmon resonance and dual polarization interferometry), are shown for only the ligands that were observed to bind. Because the binding experiments were performed by passing the same concentration of each ligand sequentially across the same protein coated chip using both techniques, the magnitude of the responses can be used to provide an approximate ranking of binding strength.

TABLE A
Ligands Identified by SPR

| | |
|---|---|
| Ligand | SPR Response Units |

Site 1

| | |
|---|---|
| 689002 | 2.3 |
| 30930 | 67.7 |
| 5069 | 67.5 |
| 7436 | 78.4 |
| 21034 | 21.1 |
| 98026 | 50 |
| 123115 | 34.5 |

Site 2

| | |
|---|---|
| 127947 | 64.8 |
| 63865 | 58 |
| 38743 | 30.6 |
| 408860 | 18.2 |
| 11891 | 15.2 |
| 156957 | 10.6 |
| 55573 | 10.6 |
| 362639 | 6.5 |
| 73735 | 1957.8 |

Site 3

| | |
|---|---|
| 93033 | 16.9 |
| 25368 | 13.3 |

Site 4

| | |
|---|---|
| 16631 | 61.8 |
| 78623 | 54.5 |
| 638134 | 25 |
| 408734 | 23.9 |
| 70980 | 14.1 |
| 68971 | 12.1 |
| 303800 | 7.8 |

Site 5

| | |
|---|---|
| 68982 | 41.2 |

Ligands Indentified by DPI

| | |
|---|---|
| Ligand | DPI Radians |

Site 1

| | |
|---|---|
| 165665 | 0.8605 |
| 164965 | 0.7494 |

Site 2

| | |
|---|---|
| 41066 | 0.2700 |
| 36914 | 0.1448 |

Site 3

| | |
|---|---|
| 80807 | 0.1909 |

Site 4

| | |
|---|---|
| 81750 | 0.4360 |
| 401077 | 0.3178 |

The inventors identified five new CD81 ligand binding sites and potential ligand binding sites on the surface of CD81-LEL using newly developed AutoDock tools, which include AutoLigand which improved the functionality of AutoDock. In addition to generating fill points for each cavity and using the collective points to provide information about the volume and depth of the cavity, properties were identified for specific point groupings (features equivalent to atoms or functional groups) that would optimize the ligand's interaction with specific atoms lining the inner surface of the cavity. Using these tools, the inventors efficiently identified new molecules that bound the protein. Previous studies using earlier versions of AutoDock that did not contain AutoLigand yielded results in which 25-55% of calculated binders actually bound to the target protein. The virtual ligand screens (docking runs) performed in this study using the new suite of tools led to the identification of 26 new small molecules that bind to CD81-LEL. Because such a high percentage of small molecules calculated by AutoDock to bind were found to bind to the protein experimentally (72%), only a small number of ligands (36) had to be tested to obtain a sufficient set of molecules for use in designing a prototype ligand conjugate for blocking E2 binding to CD81. Seven of these ligands (689002, 127947, 98026, 38743, 93033, 165665, and 164965) were also observed to exhibit similar or stronger binding (as determined by DPI) to CD81-LEL than benzyl salicylate, a small molecule reported by Holzer et al. [33] to be a moderate inhibitor blocking the binding of HCV E2 to CD81, see

TABLE B

| Ligand Added to CD81 Protein | DPI Response (Radians) |
|---|---|
| Benzyl salicylate | 0.5818 |
| 164965 | 0.7494 |
| 165665 | 0.8605 |
| 93033 | 0.7245 |
| 38743 | 0.8593 |
| 98026 | 1.1918 |
| 127947 | 0.6461 |
| 689002 | 0.438 |

TABLE C
Additional Ligands identified by SPR

| New CD81 | | |
|---|---|---|
| Ligand | Chemical Name | Smiles |
| 3001 | 2-[(3R)-1-(carboxylatomethyl)-3- | C1(CCC[CH](C1)C)(CC(═O)O) |
| | methylcyclohexyl] acetate | CC(═O)O |
| 5856 | N-[[3-(benzoylcarbamothioylamino)-4- | c1ccccc1C(NC(Nc1cc(c(cc1)C) |
| | methylphenyl]carbamothioyl]benzamide | NC(NC(c1ccccc1)═O)═S)═S)═O |
| 7962 | 3-[4-(2-methylbutan-2-yl)phenoxy]benzoate | O(c1ccc(cc1)C(CC)(C)C)c1cc |
| | | cc(c1)C(═O)O |
| 8481 | 2-(4-tert-butylphenoxy)acetate | c1c(OCC(═O)O)ccc(c1)C(C) |
| | | (C)C |
| 11667 | 4-N,6-N-bis(4-bromophenyl)-1- | Brc1ccc(cc1)Nc1nc(c2c(n1)n(nc2)C) |
| | methylpyrazolo[3,4-d]pyrimidine-4,6-diamine | Nc1ccc(Br)cc1 |
| 16162 | 2-(ethylamino)benzoate | c1(c(cccc1)NCC)C(═O)O |
| 16646 | 4-propan-2-yloxybenzoate | c1cc(ccc1OC(C)C)C(═O)O |
| 20586 | diaminomethylidene-[4- | c1c(ccc(c1)NC(═N)N)NC(N)═N |
| | (diaminomethylideneazaniumyl)phenyl]azanium | |
| 23895 | 2-carboxy-5-piperidin-1-ylphenolate | N1(CCCCC1)c1cc(c(cc1)C(═O)O)O |
| 25678 | 2-(2,4-dimethylphenyl)quinoline-4-carboxylate | n1c(c2ccc(cc2C)C)cc(c2c1cccc2) |
| | | C(═O)O |
| 31712 | 5-fluoro-2,4-dioxo-1H-pyrimidine-6- | O═c1[nH]c(═O)[nH]c(c1F)C |
| | carboxylate | (═O)O |
| 40614 | (E)-2,3-diphenylprop-2-enoate | c1ccccc1/C═C(\c1ccccc1)C |
| | | (═O)O |
| 60239 | 2-(2-prop-2-enylphenoxy)acetate | c1(c(cccc1)CC═C)OCC(═O)O |
| 73170 | (2S)-2-[(3- | Clc1cc(ccc1)NC(═O)O[CH] |
| | chlorophenyl)carbamoyloxy]propanoate | (C)C(═O)O |
| 75866 | 2-indol-1-ylacetate | n1(c2ccccc2cc1)CC(═O)O |
| 76823 | 2-(3-methoxyphenyl)-2,3-dihydro-1H- | COC1═CC═CC(═C1) |
| | perimidine | C2NC3═CC═CC4═C3C(═CC═C4)N2 |
| 87504 | 3-[(2R)-piperidin-1-ium-2-yl]pyridine | n1cc(ccc1)[CH]1NCCCC1 |
| 88883 | 3-(1,2,3,6-tetrahydropyridin-1-ium-4-yl)-1H- | c1cc2n(C3═CCNCC3)c |
| | benzimidazol-2-one | ([nH]c2cc1)═O |
| 89720 | 3-pyridin-3-ylpropanoate | n1cc(ccc1)CCC(═O)O |
| 90444 | 2-amino-3,5-dimethylbenzoate | c1c(cc(c(c1C(═O)O)N)C)C |
| 94914 | 2-[(2-phenylphenyl)methyl]propanedioate | c1ccccc1c1c(cccc1)CC |
| | | (C(═O)O)C(═O)O |
| 97538 | butyl-[[(2R)-oxolan-2-yl]methyl]azanium | O1[CH](CCC1)CNCCCC |
| 106863 | 3-oxo-6-(phenylhydrazinylidene)cyclohexa-1,4- | N(/c1ccc(cc1C(═O)O)O)═N\c |
| | diene-1-carboxylate | 1ccccc1 |
| 117922 | 3-(carbamoylcarbamoyl)pyrazine-2-carboxylate | n1c(c(ncc1)C(═O)O)C(═O)N |
| | | C(═O)N |
| 120631 | 4-(2-phenoxyethoxy)benzoate | O(c1ccc(cc1)C(═O)O)CCOc1 |
| | | ccccc1 |
| 134137 | 3-oxo-4-[(4- | N(═N\c1c(c(cc2c1cccc2)C(═O)O)O)/ |
| | sulfamoylphenyl)hydrazinylidene]naphthalene- | c1ccc(cc1)S(═O)(═O)N |
| | 2-carboxylate | |
| 142446 | 4-chloro-N-ethyl-6-(1-phenylindol-3-yl)-1,3,5- | n1(c2ccccc2)c2ccccc2c(c1)c1 |
| | triazin-2-amine | nc(Cl)nc(n1)NCC |
| 144958 | (Z)-4-oxo-4-(quinolin-8-ylamino)but-2-enoate | n1cccc2c1c(ccc2)NC |
| | | (/C═C\C(═O)O)═O |
| 148832 | (2E,4E)-5-[(1R)-1-hydroxy-2,6,6-trimethyl-4- | O═C1CC([C](C(═C1)C) |
| | oxocyclohex-2-en-1-yl]-3-methylpenta-2,4- | (/C═C/C(C)═C/C(═O)O)O)(C)C |
| | dienoate | |
| 153172 | 2-[2-(3-methoxy-4-oxocyclohexa-2,5-dien-1- | N(/c1cc(c(cc1)O)OC)═N\c1cc |
| | ylidene)hydrazinyl]benzoate | ccc1C(═O)O |
| 215276 | 4-(2,5-dimethylphenyl)-4-oxobutanoate | c1(c(cc(cc1)C)C(═O)CCC |
| | | (═O)O)C |
| 252359 | 2-(4-chlorophenyl)-5-methyl-7-(4- | c1(nc2n(c(c1)N1CCN(CC1)C) |
| | methylpiperazin-4-ium-1-yl)-[1,2,4]triazolo[1,5- | nc(n2)c1ccc(cc1)Cl)C |
| | a]pyrimidine | |
| 263636 | (3-carbamoyl-8-ethoxychromen-2- | c1c(OCC)c2oc |
| | ylidene)azanium | (c(cc2cc1)C(═O)N)═N |
| 331931 | 2-(furan-2-ylmethylamino)benzoate | c1cc(c(cc1)NCc1ccco1)C(═O)O |
| 403374 | (4-chlorophenyl)carbamothioyl- | Clc1ccc(cc1)NC(═S)NC(N)═N |
| | (diaminomethylidene)azanium | |
| 524615 | 2-[(2S)-octan-2-yl]oxycarbonylbenzoate | c1cccc(c1C(═O)O[CH](C)CC |
| | | CCCC)C(═O)O |
| 601359 | N-(4-bromonaphthalen-1-yl)-1- | c1cccc2c1c(c(cc2)C(═O)Nc1c |
| | hydroxynaphthalene-2-carboxamide | 2ccccc2c(cc1)Br)O |

### Example 2

A subset of the ligands identified to bind to CD81 have been tested using Dual Polarization Interferometry (DPI) to determine if the ligands themselves are effective in blocking E2 binding to CD81. The experiments are conducted by first immobilizing one of the proteins (HCV E2 or CD81) on the chip used to perform the assay. The other protein is then introduced to the chip and the amount of binding is measured. This protein is then washed off and the protein and one of the ligands are then introduced to the chip. If the ligand blocks/interferes with the binding of the two proteins, the DPI response (measured in Radians) is reduced compared to when the protein is added alone. After the measurement is made, the protein and ligand are washed off and the next combination of the protein and a different ligand are added to the chip and the DPI response is measured. Similar experiments can be performed in which the protein is mixed with two or more ligands to see if they have a greater effect when combined.

Experiments have been conducted with either CD81 or HCV E2 immobilized on the chip. Six ligands have been tested to date. The following Table D shows data for four ligands that block E2 binding to CD81.

TABLE D

| Molecule Added to CD81 | DPI Binding Response (Radians) |
|---|---|
| 689002 Ligand | 0.555 |
| 165665 Ligand | 0.8605 |
| 93033 Ligand | 0.7245 |
| 30930 Ligand | 1.102 |
| E2 Protein | 4.1402 |
| E2 Protein + 689002 Ligand | 2.7003 |
| E2 Protein + 165665 Ligand | −0.0047 |
| E2 Protein + 93033 Ligand | 0.2546 |
| E2 Protein + 30930 Ligand | 2.5262 |
| E2 Protein + 30930 + 68365 | 1.4531 |
| E2 Protein + 689002 + 93033 | 0.4009 |
| E2 Protein + 93033 + 165665 | 0.7654 |
| E2 Protein + 93033 + 16631 | 0.531 |

### Example 3

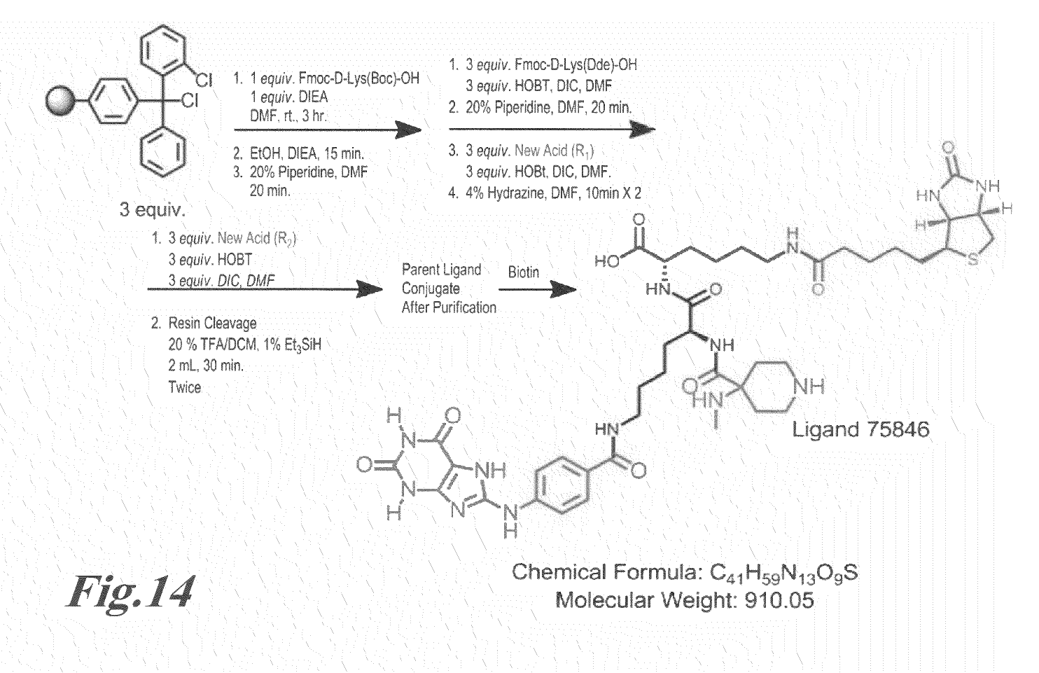

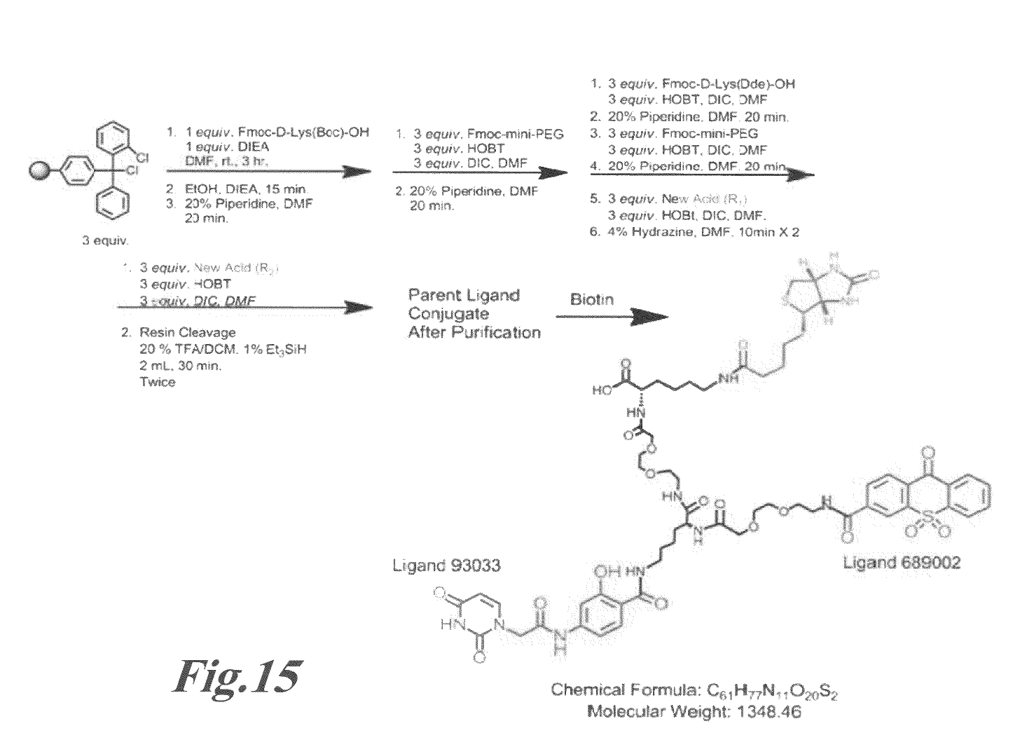

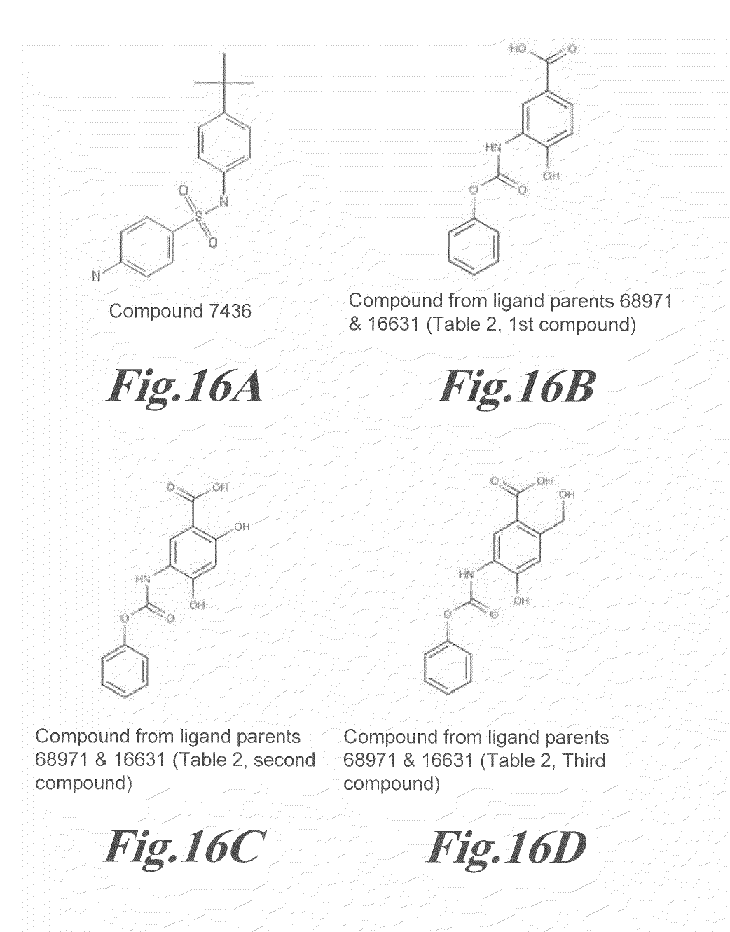

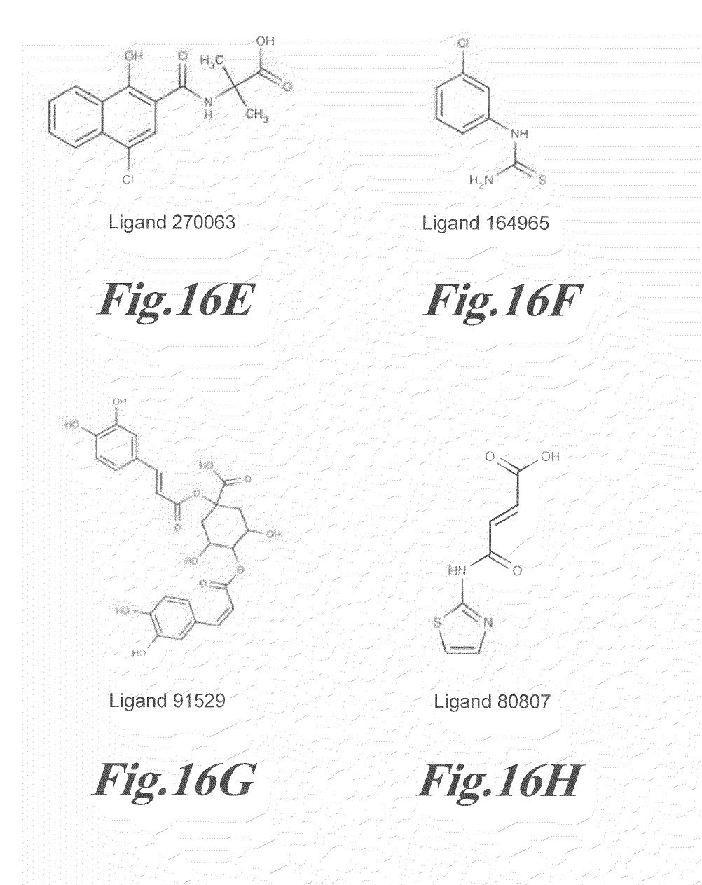

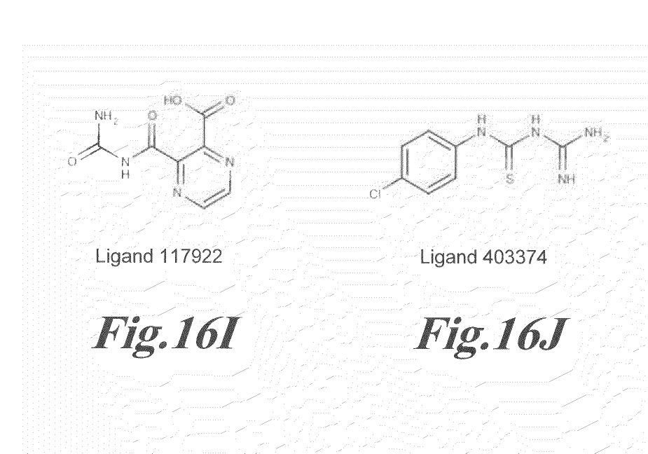

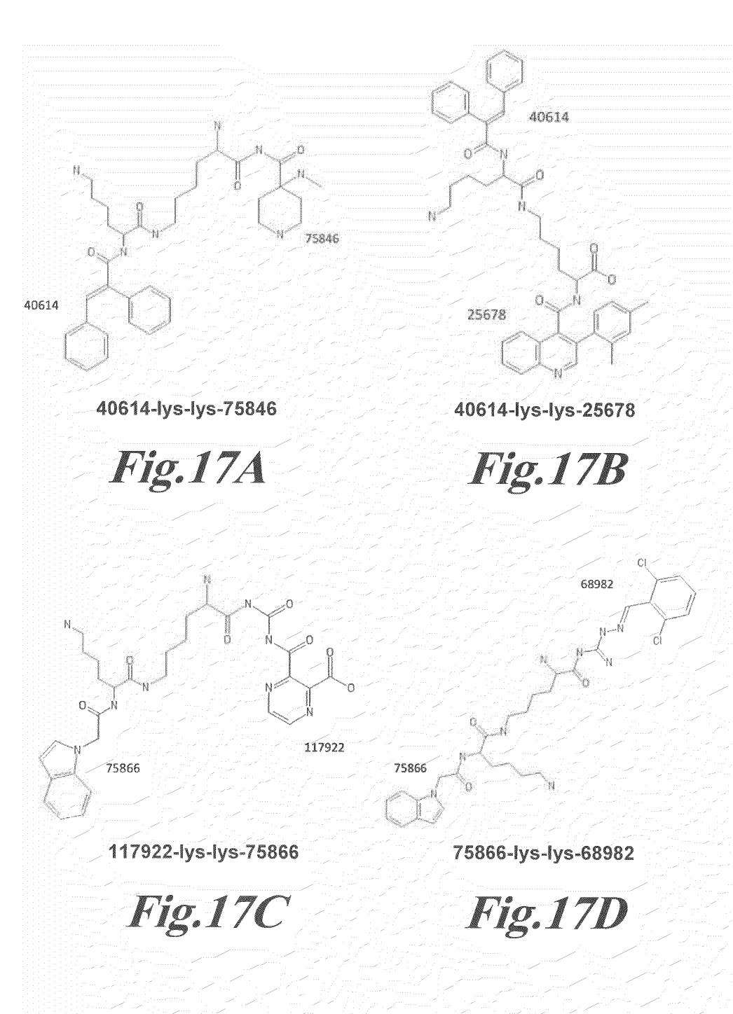

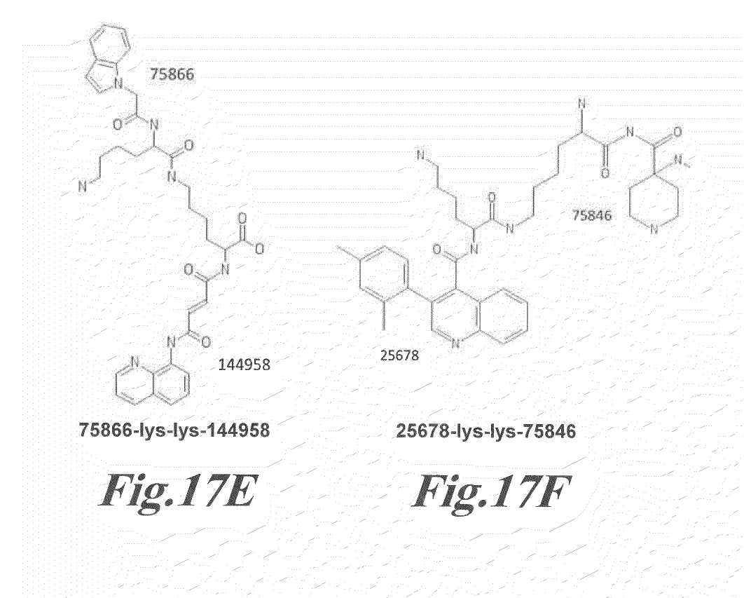

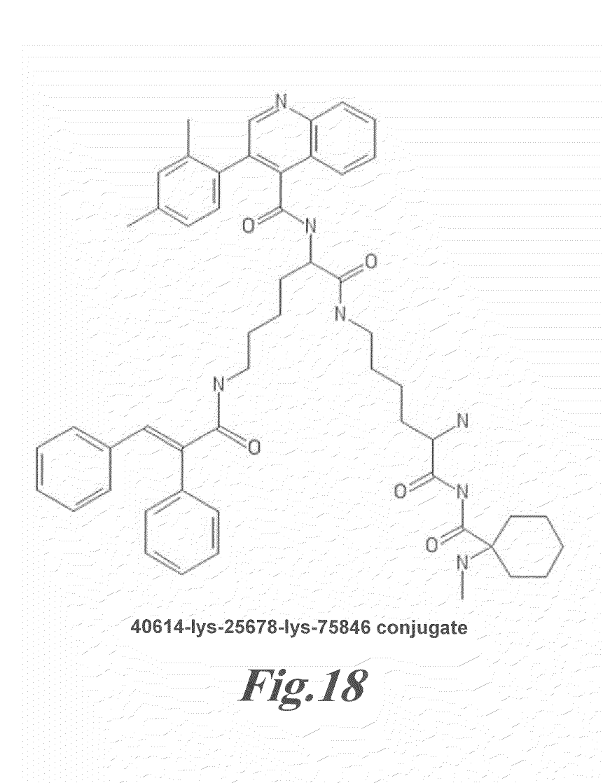

Two sets of ligand conjugates are provided as examples of specific molecules that are created by linking together either two or three of ligands that have been determined to bind to CD81. The resulting compounds function as inhibitors blocking E2 binding to CD81. The first two examples containing two ligands (see structures below) are synthesized using the procedures shown in FIGS. 14 and 15.

Two linked:

### 81750+75846 (FIG. 14)

### 93033+689002 (FIG. 15)

Similar procedures are used to link three CD81 ligands to obtain multivalent ligands described below.
Three linked:
21034+73735+68982
73735+68971+68982
70980+165665+362639

### Example 4

Screening small molecule ligands, ligand conjugates and hybrid molecules for an ability to inhibit E2 binding to CD81 or other CD81 activity in vitro.

The same technique used to screen the ligands to confirm their binding to CD81, Surface plasmon resonance (SPR), was used as an in vitro assay to test the ligands, hybrid molecules and ligand conjugates for their ability to block HCV E2 protein binding to CD81. Since the binding of E2 to CD81 is required for HCV invasion into hepatocytes, such an assay is used to identify those ligands, hybrid molecules and ligand conjugates that should be tested further. The assays are conducted using a Biacore T200 workstation (GE Healthcare, NJ, USA). The Biacore workstation is label free system that utilizes the natural phenomenon of surface plasmon resonance (SPR) to measure molecules binding to each other. The surface plasmon resonance (SPR) phenomenon occurs when polarized light strikes an electrically conducting gold layer on a biochip at the interface between media of different refractive index: the glass of a sensor surface (high refractive index) and a buffer (low refractive index (e.g., PBS buffer). A change in the resonance angle occurs when molecules bind to the surface due to the change in the refractive index of the solution close to the gold layer of the biochip.

To determine if a molecule blocks HCV E2 binding to CD81, a recombinant form of the CD81 LEL protein is immobilized for 15 min on a CM5 sensor chip at a flow speed of 5 μl/min using an amine-coupling reagent (EDC-NHS) and a competition experiment is conducted. Approximately 20,000 response units (RU) of protein is immobilized on the chip. The ligands, hybrid molecules or ligand conjugates are prepared as 600 μM solutions in PBS-0.05% Tween-80 (the running buffer) and they are introduced to the chip and binding is measured using a pre-programmed 3 minute association and 1 minute dissociation interval. To perform the competition experiment, HCV E2 protein is first added to the chip and the binding response is quantified. The HCV E2 protein is washed off, and then the ligand, hybrid molecule or ligand conjugate to be tested as a potential competitor for its ability to block HCV E2 binding is then added. After binding, the molecule is not washed off. The E2 protein is then added again, this time in the presence of the molecule being tested, and the binding response is measured. If the binding response in the presence of the ligand being tested is lower than when HCV E2 is added alone to CD81, the result indicates that the ligand being tested is effective in blocking E2 binding at the concentration tested. Such an experiment can be repeated using different concentrations of ligand to identify what concentration is required to totally block binding.

### Example 5

Screening small molecule ligands, ligand conjugates and hybrid molecules for an ability to inhibit E2 binding to CD81 or other CD81 activity in vivo.

Experiments conducted with human hepatoma cells Huh-7 are used to determine if particular ligands, hybrid molecule or ligand conjugates are effective in inhibiting HCV virus invasion by blocking E2 binding to CD81 in vivo. Huh-7 cells produce CD81 on their surface and have been shown to be a good test system for studying HCV virus invasion. The assay involves producing HCV pseudo particles in a human embryonic kidney cell line (293T) by transfecting three vectors into the kidney cells: 1) a vector encoding the retroviral Gag and Pol proteins which are responsible for particle budding at the plasma membrane and HCV RNA encapsulation, 2) a vector encoding a reporter protein (Luciferase or GFP), and 3) a vector encoding HCV glycoproteins E1 and E2, which are necessary for viral tropism and fusion of HCV pseudo type particles with the target cell membrane. The 293T cells transfected with the HCVpp are then used to infect Huh-7 cells and the extent of infection of the Huh-7 cells is evaluated by quantifying the amount of luciferase or GFP expressed in the Huh-7 cells. Working with this system, the ligands, hybrid molecules and ligand conjugates are added to the cell system at different concentrations and the level of Huh-7 infection is measured by quantifying luminescence or fluorescence produced upon Luciferase or GFP expression.

### Example 6

### Ligands Targeting CD81-LEL Identified Using SPR

Additional ligands targeting CD81 were shown to bind to CD81 using surface plasmon resonance.

Method for Determining Ligand Binding by SPR Using a Biacore Instrument

SPR analysis was performed using a Biacore T100 workstation (GE Healthcare, NJ, USA). A recombinant form of the CD81-LEL protein with a GST-tag (Shoshana Levy Lab-Stanford) was used to determine the binding affinities of the ligands in the above list. 10 uM CD81-LEL-His diluted into 10 mM sodium acetate buffer pH 4.5 was immobilized for 15 mM at a flow speed of 5 ul/min onto a CM5 sensor chip using amine coupling (EDC-NHS). Approximately 20,000 RU of protein were immobilized on the chip. The ligands were prepared as 200 uM solutions in PBS-1% DMSO (the running buffer) and they were introduced to the protein using a pre-programmed 3 min association and 1 min dissociation interval. Ligands that bind have a positive RU (response units) number and the strength of binding to the CD81 protein is related to the magnitude of the RU number; the larger the number the more binding.

TABLE E

| Ligand | M. Wt | Binding (RU) |
|---|---|---|
| 87504 | 484.51 | 57.9 |
| 40614 | 224 | 50.8 |
| 75866 | 175 | 18.9 |
| 20586 | 192 | 17.4 |
| 90444 | 165 | 13.1 |
| 89720 | 151 | 9.2 |
| 403374 | 229 | 12.6 |
| 7962 | 284 | 14.7 |
| 117922 | 210 | 10.2 |
| 106863 | 242 | 11.6 |
| 23895 | 221 | 10 |
| 25678 | 277.32 | 11.1 |
| 215276 | 206 | 7.4 |
| 120631 | 258 | 8.3 |
| 16162 | 165 | 5.1 |
| 60239 | 192 | 5.8 |
| 16646 | 180 | 4.9 |
| 331931 | 217 | 5.9 |
| 252359 | 343 | 9.3 |
| 134137 | 371 | 10 |
| 97538 | 157 | 4.2 |
| 94914 | 270 | 7.2 |
| 88883 | 215 | 5.4 |
| 31712 | 174 | 4.1 |
| 3001 | 214 | 4.6 |
| 11667 | 474 | 9.6 |
| 76823 | 292 | 5.4 |
| 73170 | 244 | 4.3 |
| 144958 | 242 | 4.1 |
| 524615 | 278 | 4.1 |
| 263636 | 232 | 3.4 |
| 148832 | 264 | 2.9 |
| 601359 | 392 | 4.1 |
| 153172 | 272 | 2.6 |
| 8481 | 208 | 1.8 |
| 142446 | 350 | 2.5 |
| 5856 | 449 | 2.7 |

A subset of these ligands and some of those listed in the patent have been tested to identify those ligands that bind to the correct region of the CD81 protein and for binding to native CD81 on living cells (a Raji cell line). This assay tests three concentrations of the ligand to determine if the ligand is capable of inhibiting the binding of an antibody called JS-81. This antibody is known to bind to the region of the CD81 protein we have targeted. If the ligand decreases JS-81 binding as you increase the concentration of the ligand, it is 1) binding to native CD81 on the Raji cells and 2) it is binding to the correct region of the protein.

### Competition Assay to Test Ligand Binding to Native CD81 on Live Cells

For antibody neutralization assay Raji cells were used, a human B cell line that expressed high amounts of CD81 on the surface (data not shown). Cells were grown in RPMI medium (10% FCS, 1% penicillin/streptomycin, 1% L-glutamine, 1% non-essential amino acids, 1% sodium pyruvate, pH 7.4, at 37° C. with 5% CO2). 2×105 cells were incubated with or without different concentrations (50 μM, 100 μM, 400 μM and 1 mM) of indicated inhibitor for 20 min at room temperature, subsequently 1 ul (16 ng/μl) of FITC-labeled anti CD81 antibody (BD Pharmingen, 551108) was added to the cells and incubated for 20 min (antibody titration was performed to obtain a working dilution range, data not shown). Cells were washed and analyzed by flow cytometry using fluorescence-activated cell sorting (FACS) (BD FACSCalibur, software: Cell Quest Pro). Mean Fluorescence Intensity MFI was calculated using Flowjo software (TreesStar, www.flowjo.com).

TABLE F
Inhibition of JS-81 antibody to CD81 on Raji Cells using different
concentrations of the ligands (50 uM, 100 uM, and 400 uM)

| | Inhibition of JS-81 Binding (%) | | |
|---|---|---|---|
| Ligand | 50 μM | 100 μM | 400 μM |
| 75866 | 0 | 1 | 31 |
| 23895 | 0 | 0 | 17 |
| 73735 | 0 | 0 | 24 |
| 7962 | 0 | 0 | 19 |
| 87504 | 0 | 6 | 20 |
| 90444 | 0 | 0 | 24 |
| 25678 | 0 | 4 | 19 |
| 40614 | 0 | 8 | 26 |
| 98026 | 0 | 0 | 21 |
| 134137 | 0 | 6 | 26 |
| 7436 | 0 | 12 | 11 |
| 30930 | 0 | 0 | 14 |
| 127947 | 0 | 0 | 24 |
| 106863 | 0 | 0 | 19 |
| 117922 | 0 | 12 | 26 |
| 144958 | 0 | 54 | 12 |
| 68982 | 0 | 13 | 15 |
| 75846 | 0 | 0 | 52 |
| 698002 | 0 | 0 | 14 |
| 93033 | 0 | 0 | 16 |

### Tables 1-37

Table AA (above) describes ligands that bind to particular CD81 binding sites. Ligand conjugates binding to two, three, four or five sites on CD81 may be designed by combining or covalently attaching ligands that bind to different CD81 sites. For example, conjugates that bind to more than one CD81 binding site may be produced by combining a ligand that binds to Site 1 with one or more ligands binding to Sites 2, 3, 4 or 5; by combining a ligand that binds to Site 2 with one or more ligands binding to Sites 3, 4, or 5; by combining a ligand that binds to Site 3, with one or more ligands binding to Sites 4 or 5; and by combining one or more ligands binding to Site 4, with one or more ligands binding to Site 5.

Table 1 (below) shows small organic molecule ligands calculated to bind to CD81.

Table 2 shows hybrid molecules designed to bind to Site 1, 2, 3 or 4 on CD81.

Tables 3-31 show representative ligands suitable for binding to particular CD81 ligands. Table 3 shows ligands for linking to compound 689002. Table 4 shows ligands for linking to compound 30930. Table 5 shows ligands for linking to compound 165665. Table 6 shows ligands for linking to compound 93033. Table 7 shows ligands for linking to compound 16631. Table 8 shows ligands for linking to compound 63865. Table 9 shows ligands for linking to compound 5069. Table 10 shows ligands for linking to compound 11891. Table 11 shows ligands for linking to compound 21034. Table 12 shows ligands for linking to compound 41066. Table 13 shows ligands for linking to compound 55573. Table 14 shows ligands for linking to compound 68971. Table 15 shows ligands for linking to compound 68982. Table 16 shows ligands for linking to compound 73735. Table 17 shows ligands for linking to compound 75846. Table 18 shows ligands for linking to compound 78623. Table 19 shows ligands for linking to compound 81750. Table 20 shows ligands for linking to compound 98026. Table 21 shows ligands for linking to compound 127947. Table 22 shows ligands for linking to compound 156957. Table 23 shows ligands for linking to compound 401077. Table 24 shows ligands for linking to compound 408734. Table 25 shows ligands for linking to compound 303800. Table 26 shows ligands for linking to compound 38743. Table 27 shows ligands for linking to compound 408860. Table 28 shows ligands for linking to compound 362639. Table 29 shows ligands for linking to compound 123115. Table 30 shows ligands for linking to compound 70980. Table 31 shows ligands for linking to compound 36914.

Tables 32-37 show other examples of ligand conjugates produced by linking different ligands binding to CD81. Table 32 shows ligands for linking to two other ligands, one chosen from each of two ligand groups shown in Tables 33, 34, 35, 36 or 37. Table 33 shows ligands for linking to two other ligands, one chosen from each of two ligand groups shown in Tables 32, 34, 35, 36 or 37. Table 34 shows ligands for linking to two other ligands, one chosen from each of two ligand groups shown in Tables 32, 33, 35, 36 or 37. Table 35 shows ligands for linking to two other ligands, one chosen from each of two ligand groups shown in Tables 32, 33, 34, 36 or 37. Table 36 shows ligands for linking to two other ligands, one chosen from each of two ligand groups shown in Tables 32, 33, 34, 35 or 37. Table 37 shows ligands for linking to two other ligands, one chosen from each of two ligand groups shown in Tables 32, 33, 34, 35 or 36.

TABLE 1

| Ligand | Chemical Name | SMILES |
|---|---|---|
| 5069 | 2-[(4S,12S,13R,14S,19R,21S)-9-nitro- | C1C[C]23[CH]4[N]1CC═1[CH](C4)[CH] |
| | 7,8-dioxo-15-oxa-1,11- | ([CH]3NC═3C2═CC |
| | diazahexacyclo[16.3.1.0\hat{ }{4,12}.0\hat{ }{4,21} | (═O)C(═O)C3N(═O)═O)[CH](OCC1) |
| | .0\hat{ }{5,10}.0\hat{ }{13,19}]docosa-5,9,17-trien- | CC(═O)O |
| | 14-yl]acetic acid | |
| 5856 | N-[[3-(benzoylcarbamothioylamino)-4- | c1ccccc1C(NC(Nc1cc(c(cc1)C)NC(NC(c |
| | methylphenyl]carbamothioyl]benzamide | 1ccccc1)═O)═S)═S)═O |
| 7436 | 4-amino-N-(4-tert-butylphenyl)benzene- | c1cc(ccc1NS(c1ccc(cc1)N)(═O)═O)C(C)(C)C |
| | 1-sulfonamide | |
| 7962 | 3-[4-(2-methylbutan-2- | O(c1ccc(cc1)C(CC)(C)C)c1cccc(c1)C(═O)O |
| | yl)phenoxy]benzoate | |
| 8481 | 2-(4-tert-butylphenoxy)acetate | c1c(OCC(═O)O)ccc(c1)C(C)(C)C |
| 11891 | 2-(1,3-benzothiazol-2-ylsulfanyl)acetic | c1cccc2c1nc(SCC(═O)O)s2 |
| | acid | |
| 16162 | 2-(ethylamino)benzoate | c1(c(cccc1)NCC)C(═O)O |
| 16631 | 3,4-dihydroxybenzoic acid | c1(cc(ccc1O)C(═O)O)O |
| 16646 | 4-propan-2-yloxybenzoate | c1cc(ccc1OC(C)C)C(═O)O |
| 20586 | diaminomethylidene-[4- | c1c(ccc(c1)NC(═N)N)NC(N)═N |
| | (diaminomethylideneazaniumyl)phenyl]azanium | |
| 21034 | (2Z)-3-(6-nitro-2H-1,3-benzodioxol-5- | O1c2cc(c(cc2OC1)/C═C\C(═O)O)N(═O)═O |
| | yl)prop-2-enoic acid | |
| 23895 | 2-carboxy-5-piperidin-1-ylphenolate | N1(CCCCC1)c1cc(c(cc1)C(═O)O)O |
| 25368 | 2-[2-hydroxy-6-(propan-2- | C1CC[CH]([CH]([CH]1OC(C)C)CC(═O) |
| | yloxy)cyclohexyl]acetic acid | O)O |
| 25678 | 2-(2,4-dimethylphenyl)quinoline-4- | n1c(c2ccc(cc2C)C)cc(c2c1cccc2)C(═O)O |
| | carboxylate | |
| 30930 | 5-(benzyloxy)-1H-indole-2-carboxylic | [nH]1c2ccc(cc2cc1C(═O)O)OCc1ccccc1 |
| | acid | |
| 31712 | 5-fluoro-2,4-dioxo-1H-pyrimidine-6- | O═c1[nH]c(═O)[nH]c(c1F)C(═O)O |
| | carboxylate | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 40614 | (E)-2,3-diphenylprop-2-enoate | c1ccccc1/C═C(\c1ccccc1)C(═O)O |
| 41066 | 2-[(2-nitrophenyl)carbamoyl]benzoic acid | c1cccc(c1C(Nc1ccccc1N(═O)═O)═O)C(═O)O |
| 55573 | (2E)-3-[(2-methylpropyl)carbamoyl]prop- | C(═C\C(═O)NCC(C)C)/C(═O)O |
| | 2-enoic acid | |
| 60239 | 2-(2-prop-2-enylphenoxy)acetate | c1(c(cccc1)CC═C)OCC(═O)O |
| 63865 | 5-{2-oxo-hexahydro-1H-thieno[3,4- | O═C1N[CH]2[CH](N1)[CH](SC2)CCCC |
| | d]imidazolidin-4-yl}pentanoic acid | C(═O)O |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 73170 | (2S)-2-[(3- | Clc1cc(ccc1)NC(═O)O[CH](C)C(═O)O |
| | chlorophenyl)carbamoyloxy]propanoate | |
| 73735 | 2-[(4-{4-[(2-carboxyphenyl)amino]-3- | c1c(ccc(c1OC)Nc1ccccc1C(═O)O)c1cc(c |
| | methoxyphenyl}-2- | (cc1)Nc1ccccc1C(═O)O)OC |
| | methoxyphenyl)amino]benzoic acid | |
| 75846 | 4-(methylamino)piperidine-4- | N1CCC(CC1)(NC)C(═O)N |
| | carboxamide | |
| 75866 | 2-indol-1-ylacetate | n1(c2ccccc2cc1)CC(═O)O |
| 78623 | 4-carbamoyl-2-(5-hydroxy-1,3-dioxo-2,3- | O═C1N(C(═O)c2c1ccc(c2)O)[CH](C(═O) |
| | dihydro-1H-isoindol-2-yl)butanoic acid | O)CCC(═O)N |
| 81750 | 4-{[(1,3-dimethyl-2,6-dioxo-2,3,6,7- | O═c1n(c(═O)c2[nH]c(nc2n1C)CNc1ccc |
| | tetrahydro-1H-purin-8- | (cc1)C(═O)O)C |
| | yl)methyl]amino}benzoic acid | |
| 87504 | 3-[(2R)-piperidin-1-ium-2-yl]pyridine | n1cc(ccc1)[CH]1NCCCC1 |
| 89720 | 3-pyridin-3-ylpropanoate | n1cc(ccc1)CCC(═O)O |
| 93033 | 4-[2-(2,4-dioxo-1,2,3,4- | O═c1n(ccc([nH]1)═O)CC(Nc1cc(c(cc1)C |
| | tetrahydropyrimidin-1-yl)acetamido]-2- | (═O)O)O)═O |
| | hydroxybenzoic acid | |
| 94914 | 2-[(2-phenylphenyl)methyl]propanedioate | c1ccccc1c1c(cccc1)CC(C(═O)O)C(═O)O |
| 97538 | butyl-[[(2R)-oxolan-2-yl]methyl]azanium | O1[CH](CCC1)CNCCCC |
| 98026 | 2-{[3-(3-methylbut-2-en-1-yl)-1,4-dioxo- | O═C1C(═C(OCC(═O)O)C(═O)c2c1cccc2) |
| | 1,4-dihydronaphthalen-2-yl]oxy}acetic | C/C═C(\C)C |
| | acid | |
| 106863 | 3-oxo-6- | N(/c1ccc(cc1C(═O)O)O)═N\c1ccccc1 |
| | (phenylhydrazinylidene)cyclohexa-1,4- | |
| | diene-1-carboxylate | |
| 117922 | 3-(carbamoylcarbamoyl)pyrazine-2- | n1c(c(ncc1)C(═O)O)C(═O)NC(═O)N |
| | carboxylate | |
| 120631 | 4-(2-phenoxyethoxy)benzoate | O(c1ccc(cc1)C(═O)O)CCOc1ccccc1 |
| 123115 | [7-methoxy-12-methyl-10,13-dioxo-11- | O═C1C2═C(N3[C]([CH]2COC(═O)N) |
| | (propylamino)-2,5-diazatetra- | ([CH]2[CH](C3)N2)OC)C(═O)C(═C1NCCC)C |
| | cyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc |
| | methoxyphenyl}-2- | (c(cc1)Nc1ccccc1C(═O)O)OC |
| | methoxyphenyl)amino]benzoic acid | |
| 134137 | 3-oxo-4-[(4- | N(═N\c1c(c(cc2c1cccc2)C(═O)O)O)/c1cc |
| | sulfamoylphenyl)hydrazinylidene]naphtha- | c(cc1)S(═O)(═O)N |
| | lene-2-carboxylate | |
| 142446 | 4-chloro-N-ethyl-6-(1-phenylindol-3-yl)- | n1(c2ccccc2)c2ccccc2c(c1)c1nc(Cl)nc |
| | 1,3,5-triazin-2-amine | (n1)NCC |
| 144958 | (Z)-4-oxo-4-(quinolin-8-ylamino)but-2- | n1cccc2c1c(ccc2)NC(/C═C\C(═O)O)═O |
| | enoate | |
| 148832 | (2E,4E)-5-[(1R)-1-hydroxy-2,6,6- | O═C1CC([C](C(═C1)C)(/C═C/C(C)═C/C |
| | trimethyl-4-oxocyclohex-2-en-1-yl]-3- | (═O)O)O)(C)C |
| | methylpenta-2,4-dienoate | |
| 153172 | 2-[2-(3-methoxy-4-oxocyclohexa-2,5- | N(/c1cc(c(cc1)O)OC)═N\c1ccccc1C(═O) |
| | dien-1-ylidene)hydrazinyl]benzoate | O |
| 156957 | 1-[(benzyloxy)carbonyl]-5- | N1([CH](CCC1═O)C(═O)O)C(OCc1cccc |
| | oxopyrrolidine-2-carboxylic acid | c1)═O |
| 165665 | (3-hydroxyphenyl)thiourea | c1ccc(cc1NC(═S)N)O |
| 215276 | 4-(2,5-dimethylphenyl)-4-oxobutanoate | c1(c(cc(cc1)C)C(═O)CCC(═O)O)C |
| 252359 | 2-(4-chlorophenyl)-5-methyl-7-(4- | c1(nc2n(c(c1)N1CCN(CC1)C)nc(n2)c1cc |
| | methylpiperazin-4-ium-1-yl)- | c(cc1)Cl)C |
| | [1,2,4]triazolo[1,5-a]pyrimidine | |
| 303800 | 3-(3-methoxy-1,2-oxazol-5-yl)propanoic | c1(onc(c1)OC)CCC(═O)O |
| | acid | |
| 331931 | 2-(furan-2-ylmethylamino)benzoate | c1cc(c(cc1)NCc1ccco1)C(═O)O |
| 362639 | 2-[2-(3,5-dioxo-2,3,4,5-tetrahydro-l,2,4- | c1cc(c2c(═O)[nH]c([nH]n2)═O)c(cc1)n1n |
| | triazin-6-yl)phenyl]-3,5-dioxo-2,3,4,5- | c(c([nH]c1═O)═O)C(═O)O |
| | tetrahydro-1,2,4-triazine-6-carboxylic acid | |
| 401077 | 2-(1,3-dioxo-2,3-dihydro-1H-isoindol-2- | O═C1N(C(═O)c2c1cccc2)[CH](C(═O)O) |
| | yl)-3-(1H-indol-3-yl)propanoic acid | Cc1c2c([nH]c1)cccc2 |
| 403374 | (4-chlorophenyl)carbamothioyl- | Clc1ccc(cc1)NC(═S)NC(N)═N |
| | (diaminomethylidene)azanium | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N |
| | | (═O)═O |
| 601359 | N-(4-bromonaphthalen-1-yl)-1- | c1cccc2c1c(c(cc2)C(═O)Nc1c2ccccc2c |
| | hydroxynaphthalene-2-carboxamide | (cc1)Br)O |
| 638134 | (2E)-2-cyano-3-(2-methoxyphenyl)prop- | c1cc(c(cc1)/C═C(/C(═O)O)C#N)OC |
| | 2-enoic acid | |
| 689002 | 9-Oxo-9H-thioxanthene-3-carboxamide | c1ccc2c(c1)C(c1c(S2(═O)═O)cc(cc1)C |
| | 10,10-dioxide | (═O)N)═O |

TABLE 2

| Ligand | | |
|---|---|---|
| Parents | Chemical Name | SMILES |
| 68971 & | 4-hydroxy-3- | C1═C(C═CC═C1)OC(NC2═CC(═CC═C2O)C(O)═O)═O |
| 16631 | [(phenoxycarbonyl)amino]benzoic acid | |
| 68971 & | 2,4-dihydroxy-5- | C1═C(C═CC═C1)OC(NC2═CC(═C(C═C2O)O)C(O)═O)═O |
| 16631 | [(phenoxycarbonyl)amino]benzoic acid | |
| 68971 & | 4-hydroxy-2-(hydroxymethyl)-5- | C1═C(C═CC═C1)OC(NC2═CC(═C(C═C2O)CO)C(O)═O)═O |
| 16631 | [(phenoxycarbonyl)amino]benzoic acid | |
| 68971 & | 5-[(5-amino-2- | C1(═C(C═C(C═C1)N)OC(NC2═CC═C(C(═C2)C(═O)O)O)═O)O |
| 78623 | hydroxyphenoxycarbonyl)amino]-2- | |
| | hydroxybenzoic acid | |
| 68971 & | 2-hydroxy-5-[(2- | C1(═C(C═CC═C1)OC(NC2═CC═C(C(═C2)C(═O)O)O)═O)O |
| 78623 | hydroxyphenoxycarbonyl)amino]benzoic | |
| | acid | |
| 78623 & | 2-(3-carbamoyl-1-carboxypropyl)-5- | O═C1N(C(═O)C2═C(C(═CC═C12)O)C(O)═O)C([H])(C(═O)O)CCC(═O)N |
| 16631 | hydroxy-1,3-dioxo-2,3-dihydro-1H- | |
| | isoindole-4-carboxylic acid | |
| 78623 & | 2-[5-amino-6-(carboxymethyl)-1,3- | O═C1N(C(═O)C2═CC(═C(C═C12)N)CC(═O)O)C([H])(C(═O)O)CCC(═O)N |
| 81750 | dioxo-2,3-dihydro-1H-isoindol-2-yl]-4- | |
| | carbamoylbutanoic acid | |
| 78623 & | 4-carbamoyl-2-[5-(carboxymethyl)-1,3- | O═C1N(C(═O)C2═CC(═CC═C12)CC(═O)O)C([H])(C(═O)O)CCC(═O)N |
| 81750 | dioxo-2,3-dihydro-1H-isoindol-2- | |
| | yl]butanoic acid | |
| 78623 & | 2-[4-(aminomethyl)-5-hydroxy-6-methyl- | O═C1N(C(═O)C2═C(C(═C(C═C12)C)O)CN)C([H])(C(═O)O)CCC(═O)N |
| 401077 | 1,3-dioxo-2,3-dihydro-1H-isoindol-2-yl]- | |
| | 4-carbamoylbutanoic acid | |
| 78623 & | 2-[4-(aminomethyl)-5-hydroxy-1,3- | O═C1N(C(═O)C2═C(C(═CC═C12)O)CN)C([H])(C(═O)O)CCC(═O)N |
| 401077 | dioxo-2,3-dihydro-1H-isoindol-2-yl]-4- | |
| | carbamoylbutanoic acid | |
| 401077 | 2-(1,3-dioxo-2,3-dihydro-1H-isoindol-2- | O═C1N(C(═O)C2═CC═CC═C12)C([H])(C(═O)O)CC3═C([N](C4═CC(═C(C═C34)O)O)[H])O |
| & 16631 | yl)-3-(2,5,6-trihydroxy-1H-indol-3- | |
| | yl)propanoic acid | |
| 401077 | 3-(5,6-dihydroxy-1H-indol-3-yl)-2-(1,3- | O═C1N(C(═O)C2═CC═CC═C12)C([H])(C(═O)O)CC3═C[N](C4═CC(═C(C═C34)O)O)[H] |
| & 16631 | dioxo-2,3-dihydro-1H-isoindol-2- | |
| | yl)propanoic acid | |
| 401077 | 3-(5-amino-1H-indol-3-yl)-2-(4-hydroxy- | O═C1N(C(═O)C2═CC═CC(═C12)O)C([H])(C(═O)O)CC3═C[N](C4═CC═C(C═C34)N)[H] |
| & 81750 | 1,3-dioxo-2,3-dihydro-1H-isoindol-2- | |
| | yl)propanoic acid | |
| 401077 | 2-(4-hydroxy-1,3-dioxo-2,3-dihydro-1H- | O═C1N(C(═O)C2═CC═CC(═C12)O)C([H])(C(═O)O)CC3═C[N](C4═CC═CC═C34)[H] |
| & 81750 | isoindol-2-yl)-3-(1H-indol-3- | |
| | yl)propanoic acid | |
| 408734 | 3-(dihydroxymethyl)-5-(hydroxymethyl)- | N1═C(N═C(C(═C1N)N═NC2═C(C═C(C═C2C(O)O)C(═O)O)CO)N)N |
| & 16631 | 4-[2-(triaminopyrimidin-5-yl)diazen-1- | |
| | yl]benzoic acid | |
| 408734 | 3-(hydroxymethyl)-4-[2- | N1═C(N═C(C(═C1N)N═NC2═C(C═C(C═C2)C(═O)O)CO)N)N |
| & 16631 | (triaminopyrimidin-5-yl)diazen-1- | |
| | yl]benzoic acid | |
| 408734 | 2-hydroxy-6-(hydroxymethyl)-4-[2- | N1═C(N═C(C(═C1N)N═NC2═CC(═C(C(═C2)CO)C(═O)O)O)N)N |
| &303800 | (triaminopyrimidin-5-yl)diazen-1- | |
| | yl]benzoic acid | |
| 408734 | 2-(hydroxymethyl)-4-[2- | N1═C(N═C(C(═C1N)N═NC2═CC═C(C(═C2)CO)C(═O)O)N)N |
| &303800 | (triaminopyrimidin-5-yl)diazen-1- | |
| | yl]benzoic acid | |
| 408734 | 2-hydroxy-4-[2-(triaminopyrimidin-5- | N1═C(N═C(C(═C1N)N═NC2═CC═C(C(═C2)O)C(═O)O)N)N |
| & 78623 | yl)diazen-1-yl]benzoic acid | |
| 81750 & | 4-{[(1,3-dimethyl-2,6-dioxo-2,3,6,7- | O═C3N(C(═O)C1═C(N═C([N]1[H])C(NC2═CC═C(C═C2)C(═O)O)O)N3C)C |
| 16631 | tetrahydro-1H-purin-8- | |
| | yl)(hydroxy)methyl]amino}benzoic acid | |
| 689002 | 6-(6-carbamoyl-9,10,10-trioxo-9H- | C1(═CC3═C(C═C1)[S](C2═CC(═CC═C2C3═O)C(═O)N)(═O)═O)C4═CC═C5C(═C4)NC(C5)C(O)═O |
| & 30930 | 10$l\hat{ }{6}-thioxanthen-2-yl)-2,3-dihydro- | |
| | 1H-indole-2-carboxylic acid | |
| 689002 | 2-(3,3-dimethylbutyl)-5,5,10-trioxo- | C3(C═CC2═C(C(C1═CC═C(C═C1[S]2(═O)═O)C(═O)N)═O)N3)CCC(C)(C)C |
| & 7436 | 1H,2H,10H-5$l\hat{ }{6},1- | |
| | thiochromeno[3,2-b]pyridine-7- | |
| | carboxamide | |
| 689002 | [6-carbamoyl-2-(2-carboxyethyl)- | C1(═CC3═C(C═C1[N](═O)O)[S](C2═CC(═CC═C2C3═O)C(═O)N)(═O)═O)CCC(O)═O |
| & 21034 | 9,10,10-trioxo-9H-10$l\hat{ }{6}-thioxanthen- | |
| | 3-yl](hydroxy)nitroso | |
| 689002 | 2-[(6-carbamoyl-2,3-diformyl-9,10,10- | C1(═C(C3═C(C═C1C═O)[S](C2═CC(═CC═C2C3═O)C(═O)N)(═O)═O)OCC(═O)O)C═O |
| & 98026 | trioxo-9H-10$l\hat{ }{6}-thioxanthen-1- | |
| | yl)oxy]acetic acid | |
| 689002 & | 9a-amino-2-(chloromethyl)-9,10,10- | C1(═CC3(C(C═C1)[S](C2═CC(═CC═C2C3═O)C(═O)N)(═O)═O)N)CC1 |
| 164965 | trioxo-9,9a-dihydro-4aH-10$l\hat{ }{6}- | |
| | thioxanthene-6-carboxamide | |
| 689002 & | 3-carbamoyl-9-methoxy-10,10-dioxo- | C1═CC3═C(C═C1)[S](C2═CC(═CC═C2C3(OC(═O)O)OC)C(═O)N)(═O)═O |
| 123115 | 9H-10$l\hat{ }{6}-thioxanthen-9-yl hydrogen | |
| | carbonate | |
| 5069 & | 2-[16-(carbamoylmethyl)-9-nitro-7,8- | C1CC45C3([H])N1CC2═CC(OC(C(C2([H])C3)([H])C4([H])NC6═C(C(C(C═C56)═O)═O)[N](═O)═O)([H])CC(═O)O)CC(N)═O |
| 689002 | dioxo-15-oxa-1,11- | |
| | diazahexacyclo[16.3.1.0\hat{ }{4,12}.0\hat{ }{4,21}.0\hat{ }{5, | |
| | 10}.0\hat{ }{13,19}]docosa-5,9,17- | |
| | trien-14-yl]acetic acid | |
| 5069 & | 2-[17-(2-aminoethyl)-9-nitro-7,8-dioxo- | C1CC45C3([H])N1CC2═C(COC(C(C2([H])C3)([H])C4([H])NC6═C(C(C(C═C56)═O)═O)[N](═O)═O)([H])CC(═O)O)CCN |
| 7436 | 15-oxa-1,11- | |
| | diazahexacyclo[16.3.1.0\hat{ }{4,12}.0\hat{ }{4,21}.0\hat{ }{5, | |
| | 10}.0\hat{ }{13,19}]docosa-5,9,17- | |
| | trien-14-yl]acetic acid | |
| 5069 & | 2-[9-nitro-6-(nitromethyl)-7,8-dioxo-15- | C1CC45C3([H])N1CC2═CCOC(C(C2([H])C3)([H])C4([H])NC6═C(C(C(C(═C56)C[N](═O)═O)═O)═O)[N](═O)═O)([H])CC(═O)O |
| 21034 | oxa-1,11- | |
| | diazahexacyclo[16.3.1.0\hat{ }{4,12}.0\hat{ }{4,21}.0\hat{ }{5, | |
| | 10}.0\hat{ }{13,19}]docosa-5,9,17- | |
| | trien-14-yl]acetic acid | |
| 5069 & | 2-[17-(2-aminoethyl)-9-nitro-6- | C1CC45C3([H])N1CC2═C(COC(C(C2([H])C3)([H])C4([H])NC6═C(C(C(C(═C56)C[N](═O)═O)═O)═O)[N](═O)═O)([H])CC(═O)O)CCN |
| 7436 & | (nitromethyl)-7,8-dioxo-15-oxa-1,11- | |
| 21034 | diazahexacyclo[16.3.1.0\hat{ }{4,12}.0\hat{ }{4,21}.0\hat{ }{5, | |
| | 10}.0\hat{ }{13,19}]docosa-5,9,17- | |
| | trien-14-yl]acetic acid | |
| 30930 & | 5-{[2-(carbamothioylamino)-3- | [N]3([H])C1═C(C═C(C═C1)OCC2═CC═CC(═C2NC(N)═S)C═O)C═C3C(═O)O |
| 165665 | formylphenyl]methoxy}-1H-indole-2- | |
| | carboxylic acid | |
| 30930 & | [4-(aminomethyl)-2-{[(2-carboxy-7- | [N]3([H])C1═C(C═C(C═C1C)OCC2═C(C═CC(═C2)CN)[S](═O)═O)C═C3C(═O)O |
| 7436 | methyl-1H-indol-5- | |
| | yl)oxy]methyl}benzene]sulfonyl | |
| 30930 & | 5-(2-nitro-1-phenylethoxy)-1H-indole-2- | [N]3([H])C1═C(C═C(C═C1)OC(C2═CC═CC═C2)C[N](═O)═O)C═C3C(═O)O |
| 21034 | carboxylic acid | |
| 30930 & | 5-(3-oxo-1-phenylpropoxy)-1H-indole-2- | [N]3([H])C1═C(C═C(C═C1)OC(C2═CC═CC═C2)CC═O)C═C3C(═O)O |
| 98026 | carboxylic acid | |
| 30930 & | 5-{2-[(4-amino-2,5-dioxocyclohex-3-en- | [N]4([H])C1═C(C═C(C═C1)OC(C2═CC═CC═C2)CNC3CC(C(═CC3═O)N)═O)C═C4C(═O)O |
| 123115 | 1-yl)amino]-1-phenylethoxy}-1H-indole- | |
| | 2-carboxylic acid | |
| 165665 | {3-[(4-tert-butylphenyl)sulfamoyl]-5- | C1═C(C═C(C═C1[S](NC2═CC═C(C═C2)C(C)(C)C)(═O)═O)O)NC(═S)N |
| & 7436 | hydroxyphenyl}thiourea | |
| 165665 | 3-{2-[3-(carbamothioylamino)-5- | C1═C(C═C(C═C1C2CC3C(C2)C═C(C(═C3)[N](═O)═O)CCC(═O)O)O)NC(═S)N |
| & 21034 | hydroxyphenyl]-6-nitro-2,3,3a,7a- | |
| | tetrahydro-1H-inden-5-yl}propanoic acid | |
| 165665 | 2-[(3-{2-[2-(carbamothioylamino)-4- | C1(═C(C═C(C═C1)O)NC(═S)N)CCC2═C(C(C3C(C2═O)C═CC═C3)═O)OCC(O)═O |
| & 98026 | hydroxyphenyl]ethyl}-1,4-dioxo- | |
| | 1,4,4a,8a-tetrahydronaphthalen-2- | |
| | yl)oxy]acetic acid | |
| 165665 & | (3-{amino[(3- | C1(═CC(═CC(═C1)C(NC2═CC(═CC═C2)Cl)N)NC(N)═S)O |
| 164965 | chlorophenyl)amino]methyl}-5- | |
| | hydroxyphenyl)thiourea | |
| 7436 & | 4-amino-N-(4-tert-butyl-2-hydroxy-5- | C1(═C(C═C(C(═C1)C(C)(C)C)[N](═O)═O)N[S](C2═CC═C(C═C2)N)(═O)═O)O |
| 21034 | nitrophenyl)benzene-1-sulfonamide | |
| 7436 & | 2-[(4-aminobenzene)sulfonamido]-5-tert- | C1(═C(C═C(C(═C1)C(C)(C)C)C═O)N[S](C2═CC═C(C═C2)N)(═O)═O)OC(═O)O |
| 98026 | butyl-4-formylphenyl hydrogen | |
| | carbonate | |
| 7436 & | 2-{4-[(2,4- | C1═C(C═CC(═C1)C(C(O)═O)(C)C)N[S](C2═C(C═C(C═C2)N)N)(═O)═O |
| 164965 | diaminobenzene)sulfonamido]phenyl}-2- | |
| | methylpropanoic acid | |
| 7436 & | [7-(2-aminoethoxy)-4-(4-tert- | O═C5C1═C(N2C(C1([H])COC(═O)N)(C3([H])C(C2)(N3)C4═CC═C(C═C4)C(C)(C)C)OCCN)C(═O)C(═C5NCCC)C |
| 123115 | butylphenyl)-12-methyl-10,13-dioxo-11- | |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 21034 & | 3-(3a-amino-6-nitro-3a,7a-dihydro-2H- | O2C1C═C(C(═CC1(OC2)N)C(═CC(═O)O)C1)[N](═O)═O |
| 164965 | 1,3-benzodioxol-5-yl)-3-chloroprop-2- | |
| | enoic acid | |
| 21034 & | 3-(2-{2-[(carbamoyloxy)methyl]-5- | O2C1═C(C═C(C(═C1)[N](═O)═O)C═CC(═O)O)OC2C3═C(C(C(C(C3═O)C)NCCC)═O)COC(N)═O |
| 123115 | methyl-3,6-dioxo-4- | |
| | (propylamino)cyclohex-1-en-1-yl}-6- | |
| | nitro-2H-1,3-benzodioxol-5-yl)prop-2- | |
| | enoic acid | |
| 98026 & | 2-{[3-(2-hydrazinyl-3-methylbut-2-en-1- | O═C1C(═C(OCC(═O)O)C(═O)C2═CC═CC═C12)CC(═C(C)C)NN |
| 164965 | yl)-1,4-dioxo-1,4-dihydronaphthalen-2- | |
| | yl]oxy}acetic acid | |
| 98026 & | 2-({3-[1-({6-[2- | O═C1C(═C(OCC(═O)O)C(═O)C2═CC═CC═C12)C(C═C(C)C)NC3C(C(C(═C(C3═O)C)NC)═O)═CCOC(N)═O |
| 123115 | (carbamoyloxy)ethylidene]-3-methyl-4- | |
| | (methylamino)-2,5-dioxocyclohex-3-en- | |
| | 1-yl}amino)-3-methylbut-2-en-1-yl]-1,4- | |
| | dioxo-1,4-dihydronaphthalen-2- | |
| | yl}oxy)acetic acid | |
| 123115 & | [4-(3-chlorophenyl)-7-methoxy-12- | O═C5C1═C(N2C(C1([H])COC(═O)N)(C3([H])C(C2)(N3)C4═CC(═CC═C4)Cl)OC)C(═O)C(═C5NCCC)C |
| 164965 | methyl-10,13-dioxo-11-(propylamino)- | |
| | 2,5- | |
| | diazatetracyclo[7.4.0.0\hat{ }{2,7}.0\hat{ }{4,6}]trideca- | |
| | 1(9),11-dien-8-yl]methyl carbamate | |
| 156957 & | 1-[({2- | N1(C([H])(CCC1═O)C(═O)O)C(OCC2═CC═CC═C2CNO)═O |
| 127947 | [(hydroxyamino)methyl]phenyl}methoxy)carbonyl]- | |
| | 5-oxopyrrolidine-2-carboxylic | |
| | acid | |
| 156957 & | 1-[({2-[(hydroxyamino)methyl]-6-(2- | N1(C([H])(CCC1═O)C(═O)O)C(OCC2═C(C═CC═C2CNO)CCO)═O |
| 127947 | hydroxyethyl)phenyl}methoxy)carbonyl]- | |
| | 5-oxopyrrolidine-2-carboxylic acid | |
| 156957 | 1-[(benzyloxy)carbonyl]-4-(4-{[2- | N1(C([H])(CC(C1═O)C2═CC═C(C(═C2)OC)NC3═CC═CC═C3C(O)O)C(═O)O)C(OCC4═CC═CC═C4)═O |
| & 73735 | (dihydroxymethyl)phenyl]amino}-3- | |
| | methoxyphenyl)-5-oxopyrrolidine-2- | |
| | carboxylic acid | |
| 156957 | [2-(3-{[(2-carboxy-5-oxopyrrolidin-1- | N1(C([H])(CCC1═O)C(═O)O)C(OCC2═CC(═CC═C2)CC[N](O)═O)═O |
| & 41066 | yl)carbonyloxy]methyl}phenyl)ethyl](hydroxy)- | |
| | oxo-$1\hat{ }{5}-azanyl | |
| 156957 | 1-[({3- | N1(C([H])(CCC1═O)C(═O)O)C(OCC2═CC═CC(═C2)C(O)N)═O |
| & 63865 | [amino(hydroxy)methyl]phenyl}methoxy)carbonyl]- | |
| | 5-oxopyrrolidine-2-carboxylic | |
| | acid | |
| 156957 | 1-{[(2- | N1(C([H])(CCC1═O)C(═O)O)C(OCC2═CC═CC═C2C(═O)N)═O |
| & 38743 | carbamoylphenyl)methoxy]carbonyl}-5- | |
| | oxopyrrolidine-2-carboxylic acid | |
| 156957 & | 2-(3-{[(2-carboxy-5-oxopyrrolidin-1- | N1(C([H])(CCC1═O)C(═O)O)C(OCC2═CC═CC(═C2)N3C(NC(C(N3)C(═O)O)O)O)═O |
| 362639 | yl)carbonyloxy]methyl}phenyl)-3,5- | |
| | dihydroxy-1,2,4-triazinane-6-carboxylic | |
| | acid | |
| 127947 | 2-[(4-{4-[(2-carboxyphenyl)amino]-3- | C1═C(C(═CC═C1C2═CC═C(C(═C2)OC)NC3═C(C═CC(═C3)C(C)NO)C(═O)O)NC4═C(C═CC═C4)C(═O)O)OC |
| & 73735 | methoxyphenyl}-2- | |
| | methoxyphenyl)amino]-4-[1- | |
| | (hydroxyamino)ethyl]benzoic acid | |
| 73735 & | 2-({4-[2-(carboxymethyl)-4-[(2- | C1═C(C(═CC═C1C2═C(C═C(C(═C2)OC)NC3═C(C═CC═C3)C═O)CC(═O)O)NC4═C(C═CC═C4)C(═O)O)OC |
| 55573 | formylphenyl)amino]-5-methoxyphenyl]- | |
| | 2-methoxyphenyl}amino)benzoic acid | |
| 73735 & | 2-[(2-methoxy-4-{3-methoxy-4-[(2- | C1═C(C(═CC═C1C2═CC═C(C(═C2)OC)NC3═C(C═CC═C3)[N](═O)═O)NC4═C(C═CC═C4)C(═O)O)OC |
| 41066 | nitrophenyl)amino]phenyl}phenyl)amino]benzoic | |
| | acid | |
| 73735 & | 2-({4-[3-(2,3,3a,7a-tetrahydro-1,3- | C1═C(C(═CC═C1C2═CC(═C(C(═C2)OC)N)C4SC3C(C═CC═C3)N4)NC5═C(C═CC═C5)C(═O)O)OC |
| 11891 | benzothiazol-2-yl)-4-amino-5- | |
| | methoxyphenyl]-2- | |
| | methoxyphenyl}amino)benzoic acid | |
| 73735 & | 2-({4-[3-(1-carboxy-2-{2-oxo- | C1═C(C(═CC═C1C2═CC(═CC(═C2)OC)C(CC3C4C(SC3)NC(N4)═O)C(O)═O)NC5═C(C═CC═C5)C(═O)O)OC |
| 63865 | hexahydro-1H-thieno[2,3- | |
| | d]imidazolidin-6-yl}ethyl)-5- | |
| | methoxyphenyl]-2- | |
| | methoxyphenyl}amino)benzoic acid | |
| 73735 & | 2-[(4-{4-[(2-carbamoyl-6- | C1═C(C(═CC═C1C2═CC═C(C(═C2)OC)NC3═C(C═CC═C3C(N)═O)C═O)NC4═C(C═CC═C4)C(═O)O)OC |
| 38743 | formylphenyl)amino]-3- | |
| | methoxyphenyl}-2- | |
| | methoxyphenyl)amino]benzoic acid | |
| 73735 & | 2-{[4-(3-{[2-(6-formyl-3,5-dioxo- | C1═C(C(═CC═C1C2═CC(═CC(═C2)OC)NC(═O)C3═CC═CC═C3N4C(NC(C(═N4)C═O)═O)═O)NC5═C(C═CC═C5)C(═O)O)OC |
| 362639 | 2,3,4,5-tetrahydro-1,2,4-triazin-2- | |
| | yl)benzene]amido}-5-methoxyphenyl)-2- | |
| | methoxyphenyl]amino}benzoic acid | |
| 73735 & | 2-[(2-methoxy-4-{3-methoxy-5-[nitro(6- | C1═C(C(═CC═C1C2═CC(═CC(═C2)OC)C([N](═O)═O)N3C4C(NC3)N═CNC4═S)NC5═C(C═CC═C5)C(═O)O)OC |
| 36914 | sulfanylidene-4,5,6,7,8,9-hexahydro-1H- | |
| | purin-7- | |
| | yl)methyl]phenyl}phenyl)amino]benzoic | |
| | acid | |
| 362639 | 2-{2-[4-(nitromethyl)-3,5-dioxo-2,3,4,5- | C1═CC═C(C(═C1)C2═NN(C(N(C2═O)C[N](═O)═O)═O)[H])N3N═C(C(N([H])C3═O)═O)C(═O)O |
| & 55573 | tetrahydro-1,2,4-triazin-6-yl]phenyl}-3,5- | |
| | dioxo-2,3,4,5-tetrahydro-1,2,4-triazine-6- | |
| | carboxylic acid | |
| 362639 | 3-(2,5-dioxo-4-phenylimidazolidin-4-yl)- | O═C2NC(C1═CC═CC═C1)(C(═O)N2)C(CC(═O)O)[N](═O)═O |
| & 41066 | 3-nitropropanoic acid | |
| 362639 | 2-({7-[(6-formyl-3,5-dioxo-2,3,4,5- | C1═C3C(═C(C═C1)CN2C(NC(C(═N2)C═O)═O)═O)SC(═N3)SCC(═O)O |
| & 11891 | tetrahydro-1,2,4-triazin-2-yl)methyl]-1,3- | |
| | benzothiazol-2-yl}sulfanyl)acetic acid | |
| 63865 & | 4-nitro-5-{2-oxo-hexahydro-1H- | O═C1NC2([H])C([H])(N1)C([H])(SC2)CC(CCC(═O)O)[N](═O)═O |
| 41066 | thieno[3,4-d]imidazolidin-4-yl}pentanoic | |
| | acid | |
| 63865 & | 4-formamido-5-{2-oxo-hexahydro-1H- | O═C1NC2([H])C([H])(N1)C[H](SC2)CC(CCC(═O)O)NC═O |
| 38743 | thieno[3,4-d]imidazolidin-4-yl}pentanoic | |
| | acid | |
| 93033 & | 2-hydroxy-4-{2-[2-(2-hydroxyphenyl)-4- | C1(N(C═CC(N1[H])═O)CC(NC2═CC═C(C(═C2)O)C(═O)O)═O)C3═CC═CC═C3O |
| 25368 | oxo-1,2,3,4-tetrahydropyrimidin-1- | |
| | yl]acetamido}benzoic acid | |
| 408860 | 4-(2-amino-2-carbamoylethyl)-2-[(3- | C1═C(C(═CC(═C1)CC(C(N)═O)N)C(═O)NC2═CC═CC(═C2)[N](═O)═O)C(═O)O |
| & 75846 | nitrophenyl)carbamoyl]benzoic acid | |

TABLE 3

| Ligand | Chemical Name | SMILES |
|---|---|---|
| 16631 | 3,4-dihydroxybenzoic acid | c1(cc(ccc1O)C(═O)O)O |
| 25368 | 2-[2-hydroxy-6-(propan-2- | C1CC[CH]([CH]([CH]1OC(C)C)CC(═O)O)O |
| | yloxy)cyclohexyl]acetic acid | |
| 68971 | 2-hydroxy-5- | c1ccccc1OC(Nc1cc(c(cc1)O)C(═O)O)═O |
| | [(phenoxycarbonyl)amino]benzoic acid) | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 93033 | 4-[2-(2,4-dioxo-1,2,3,4- | O═c1n(ccc([nH]1)═O)CC(Nc1cc(c(cc1)C(═O)O)O)═O |
| | tetrahydropyrimidin-1-yl)acetamido]-2- | |
| | hydroxybenzoic acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |
| New | | |
| CD81 | | |
| Ligand | Chemical Name | Smiles |
| 5856 | N-[[3-(benzoylcarbamothioylamino)-4- | c1ccccc1C(NC(Nc1cc(c(cc1)C)NC(NC(c1ccccc1)═O)═S)═S)═O |
| | methylphenyl]carbamothioyl]benzamide | |
| 8481 | 2-(4-tert-butylphenoxy)acetate | c1c(OCC(═O)O)ccc(c1)C(C)(C)C |
| 16162 | 2-(ethylamino)benzoate | c1(c(cccc1)NCC)C(═O)O |
| 20586 | diaminomethylidene-[4- | c1c(ccc(c1)NC(═N)N)NC(N)═N |
| | (diaminomethylideneazaniumyl)phenyl]azanium | |
| 25678 | 2-(2,4-dimethylphenyl)quinoline-4- | n1c(c2ccc(cc2C)C)cc(c2c1cccc2)C(═O)O |
| | carboxylate | |
| 60239 | 2-(2-prop-2-enylphenoxy)acetate | c1(c(cccc1)CC═C)OCC(═O)O |
| 75866 | 2-indol-1-ylacetate | n1(c2ccccc2cc1)CC(═O)O |
| 87504 | 3-[(2R)-piperidin-1-ium-2-yl]pyridine | n1cc(ccc1)[CH]1NCCCC1 |
| 89720 | 3-pyridin-3-ylpropanoate | n1cc(ccc1)CCC(═O)O |
| 215276 | 4-(2,5-dimethylphenyl)-4-oxobutanoate | c1(c(cc(cc1)C)C(═O)CCC(═O)O)C |
| 331931 | 2-(furan-2-ylmethylamino)benzoate | c1cc(c(cc1)NCc1ccco1)C(═O)O |
| 403374 | (4-chlorophenyl)carbamothioyl- | Clc1ccc(cc1)NC(═S)NC(N)═N |
| | (diaminomethylidene)azanium | |

TABLE 4

| Ligand | Chemical Name | SMILES |
|---|---|---|
| 11891 | 2-(1,3-benzothiazol-2-ylsulfanyl)acetic | c1cccc2c1nc(SCC(═O)O)s2 |
| | acid | |
| 16631 | 3,4-dihydroxybenzoic acid | c1(cc(ccc1O)C(═O)O)O |
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 55573 | (2E)-3-[(2-methylpropyl)carbamoyl]prop- | C(═C\C(═O)NCC(C)C)/C(═O)O |
| | 2-enoic acid | |
| 63865 | 5-{2-oxo-hexahydro-1H-thieno[3,4- | O═C1N[CH]2[CH](N1)[CH](SC2)CCCCC(═O)O |
| | d]imidazolidin-4-yl}pentanoic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |

TABLE 5

| Ligand | Chemical Name | SMILES |
|---|---|---|
| 11891 | 2-(1,3-benzothiazol-2-ylsulfanyl)acetic | c1cccc2c1nc(SCC(═O)O)s2 |
| | acid | |
| 16631 | 3,4-dihydroxybenzoic acid | c1(cc(ccc1O)C(═O)O)O |
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 41066 | 2-[(2-nitrophenyl)carbamoyl]benzoic acid | c1cccc(c1C(Nc1ccccc1N(═O)═O)═O)C(═O)O |
| 55573 | (2E)-3-[(2-methylpropyl)carbamoyl]prop- | C(═C\C(═O)NCC(C)C)/C(═O)O |
| | 2-enoic acid | |
| 63865 | 5-{2-oxo-hexahydro-1H-thieno[3,4- | O═C1N[CH]2[CH](N1)[CH](SC2)CCCCC(═O)O |
| | d]imidazolidin-4-yl}pentanoic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 93033 | 4-[2-(2,4-dioxo-1,2,3,4- | O═c1n(ccc([nH]1)═O)CC(Nc1cc(c(cc1)C(═O)O)O)═O |
| | tetrahydropyrimidin-1-yl)acetamido]-2- | |
| | hydroxybenzoic acid | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |

TABLE 6

| Ligand | Chemical Name | SMILES |
|---|---|---|
| 5069 | 2-[(4S,12S,13R,14S,19R,21S)-9-nitro- | C1C[C]23[CH]4[N]1CC═1[CH](C4)[CH]- |
| | 7,8-dioxo-15-oxa-1,11- | ([CH]3NC═3C2═CC(═O)C(═O)C3N(═O)═O)[CH](OCC1)CC(═O)O |
| | diazahexacyclo[16.3.1.0\hat{ }{4,12}.0\hat{ }{4,21}. | |
| | 0\hat{ }{5,10}.0\hat{ }{13,19}]docosa-5,9,17-trien- | |
| | 14-yl]acetic acid | |
| 11891 | 2-(1,3-benzothiazol-2-ylsulfanyl)acetic | c1cccc2c1nc(SCC(═O)O)s2 |
| | acid | |
| 21034 | (2Z)-3-(6-nitro-2H-1,3-benzodioxol-5- | O1c2cc(c(cc2OC1)/C═C\C(═O)O)N(═O)═O |
| | yl)prop-2-enoic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |
| New | | |
| CD81 | | |
| Ligand | Chemical Name | Smiles |
| 16162 | 2-(ethylamino)benzoate | c1(c(cccc1)NCC)C(═O)O |
| 23895 | 2-carboxy-5-piperidin-1-ylphenolate | N1(CCCCC1)c1cc(c(cc1)C(═O)O)O |
| 89720 | 3-pyridin-3-ylpropanoate | n1cc(ccc1)CCC(═O)O |
| 142446 | 4-chloro-N-ethyl-6-(1-phenylindol-3-yl)- | n1(c2ccccc2)c2ccccc2c(c1)c1nc(Cl)nc(n1)NCC |
| | 1,3,5-triazin-2-amine | |
| 148832 | (2E,4E)-5-[(1R)-1-hydroxy-2,6,6- | O═C1CC([C](C(═C1)C)(/C═C/C(C)═C/C(═O)O)O)(C)C |
| | trimethyl-4-oxocyclohex-2-en-1-yl]-3- | |
| | methylpenta-2,4-dienoate | |
| 215276 | 4-(2,5-dimethylphenyl)-4-oxobutanoate | c1(c(cc(cc1)C)C(═O)CCC(═O)O)C |
| 252359 | 2-(4-chlorophenyl)-5-methyl-7-(4- | c1(nc2n(c(c1)N1CCN(CC1)C)nc(n2)c1ccc(cc1)Cl)C |
| | methylpiperazin-4-ium-1-yl)- | |
| | [1,2,4]triazolo[1,5-a]pyrimidine | |
| 601359 | N-(4-bromonaphthalen-1-yl)-1- | c1cccc2c1c(c(cc2)C(═O)Nc1c2ccccc2c(cc1)Br)O |
| | hydroxynaphthalene-2-carboxamide | |
| 7962 | 3-[4-(2-methylbutan-2- | O(c1ccc(cc1)C(CC)(C)C)c1cccc(c1)C(═O)O |
| | yl)phenoxy]benzoate | |
| 16646 | 4-propan-2-yloxybenzoate | c1cc(ccc1OC(C)C)C(═O)O |
| 23895 | 2-carboxy-5-piperidin-1-ylphenolate | N1(CCCCC1)c1cc(c(cc1)C(═O)O)O |
| 31712 | 5-fluoro-2,4-dioxo-1H-pyrimidine-6- | O═c1[nH]c(═O)[nH]c(c1F)C(═O)O |
| | carboxylate | |
| 73170 | (2S)-2-[(3- | Clc1cc(ccc1)NC(═O)O[CH](C)C(═O)O |
| | chlorophenyl)carbamoyloxy]propanoate | |
| 94914 | 2-[(2-phenylphenyl)methyl]propanedioate | c1ccccc1c1c(cccc1)CC(C(═O)O)C(═O)O |
| 97538 | butyl-[[(2R)-oxolan-2-yl]methyl]azanium | O1[CH](CCC1)CNCCCC |
| 106863 | 3-oxo-6- | N(/c1ccc(cc1C(═O)O)O)═N\c1ccccc1 |
| | (phenylhydrazinylidene)cyclohexa-1,4- | |
| | diene-1-carboxylate | |
| 117922 | 3-(carbamoylcarbamoyl)pyrazine-2- | n1c(c(ncc1)C(═O)O)C(═O)NC(═O)N |
| | carboxylate | |
| 120631 | 4-(2-phenoxyethoxy)benzoate | O(c1ccc(cc1)C(═O)O)CCOc1ccccc1 |
| 134137 | 3-oxo-4-[(4- | N(═N\c1c(c(cc2c1cccc2)C(═O)O)O)/c1ccc(cc1)S(═O)(═O)N |
| | sulfamoylphenyl)hydrazinylidene]naphthalene- | |
| | 2-carboxylate | |
| 144958 | (Z)-4-oxo-4-(quinolin-8-ylamino)but-2- | n1cccc2c1c(ccc2)NC(/C═C\C(═O)O)═O |
| | enoate | |
| 153172 | 2-[2-(3-methoxy-4-oxocyclohexa-2,5- | N(/c1cc(c(cc1)O)OC)═N\c1ccccc1C(═O)O |
| | dien-1-ylidene)hydrazinyl]benzoate | |
| 252359 | 2-(4-chlorophenyl)-5-methyl-7-(4- | c1(nc2n(c(c1)N1CCN(CC1)C)nc(n2)c1ccc(cc1)Cl)C |
| | methylpiperazin-4-ium-1-yl)- | |
| | [1,2,4]triazolo[1,5-a]pyrimidine | |

TABLE 7

| Ligand | Chemical Name | SMILES |
|---|---|---|
| 5069 | 2-[(4S,12S,13R,14S,19R,21S)-9-nitro- | C1C[C]23[CH]4[N]1CC═1[CH](C4)[CH]- |
| | 7,8-dioxo-15-oxa-1,11- | ([CH]3NC═3C2═CC(═O)C(═O)C3N(═O)═O)[CH](OCC1)CC(═O)O |
| | diazahexacyclo[16.3.1.0\hat{ }{4,12}.0\hat{ }{4,21}. | |
| | 0\hat{ }{5,10}.0\hat{ }{13,19}]docosa-5,9,17-trien- | |
| | 14-yl]acetic acid | |
| 11891 | 2-(1,3-benzothiazol-2-ylsulfanyl)acetic | c1cccc2c1nc(SCC(═O)O)s2 |
| | acid | |
| 21034 | (2Z)-3-(6-nitro-2H-1,3-benzodioxol-5- | O1c2cc(c(cc2OC1)/C═C\C(═O)O)N(═O)═O |
| | yl)prop-2-enoic acid | |
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 41066 | 2-[(2-nitrophenyl)carbamoyl]benzoic acid | c1cccc(c1C(Nc1ccccc1N(═O)═O)═O)C(═O)O |
| 55573 | (2E)-3-[(2-methylpropyl)carbamoyl]prop- | C(═C\C(═O)NCC(C)C)/C(═O)O |
| | 2-enoic acid | |
| 68982 | 1-[(E)-[(2,6- | Clc1c(c(Cl)ccc1)/C═N/NC(N)═N |
| | dichlorophenyl)methylidene]amino]guanidine | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 408860 | 2-[(3-nitrophenyl)carbamoyl]benzoic acid | c1cccc(c1C(═O)O)C(═O)Nc1cc(ccc1)N(═O)═O |
| New | | |
| CD81 | | |
| Ligand | Chemical Name | Smiles |
| 5856 | N-[[3-(benzoylcarbamothioylamino)-4- | c1ccccc1C(NC(Nc1cc(c(cc1)C)NC(NC(c1ccccc1)═O)═S)═S)═O |
| | methylphenyl]carbamothioyl]benzamide | |
| 7962 | 3-[4-(2-methylbutan-2- | O(c1ccc(cc1)C(CC)(C)C)c1cccc(c1)C(═O)O |
| | yl)phenoxy]benzoate | |
| 8481 | 2-(4-tert-butylphenoxy)acetate | c1c(OCC(═O)O)ccc(c1)C(C)(C)C |
| 16646 | 4-propan-2-yloxybenzoate | c1cc(ccc1OC(C)C)C(═O)O |
| 20586 | diaminomethylidene-[4- | c1c(ccc(c1)NC(═N)N)NC(N)═N |
| | (diaminomethylideneazaniumyl)phenyl]azanium | |
| 23895 | 2-carboxy-5-piperidin-1-ylphenolate | N1(CCCCC1)c1cc(c(cc1)C(═O)O)O |
| 75866 | 2-indol-1-ylacetate | n1(c2ccccc2cc1)CC(═O)O |
| 87504 | 3-[(2R)-piperidin-1-ium-2-yl]pyridine | n1cc(ccc1)[CH]1NCCCC1 |
| 142446 | 4-chloro-N-ethyl-6-(1-phenylindol-3-yl)- | n1(c2ccccc2)c2ccccc2c(c1)c1nc(Cl)nc(n1)NCC |
| | 1,3,5-triazin-2-amine | |
| 148832 | (2E,4E)-5-[(1R)-1-hydroxy-2,6,6- | O═C1CC([C](C(═C1)C)(/C═C/C(C)═C/C(═O)O)O)(C)C |
| | trimethyl-4-oxocyclohex-2-en-1-yl]-3- | |
| | methylpenta-2,4-dienoate | |
| 252359 | 2-(4-chlorophenyl)-5-methyl-7-(4- | c1(nc2n(c(c1)N1CCN(CC1)C)nc(n2)c1ccc(cc1)Cl)C |
| | methylpiperazin-4-ium-1-yl)- | |
| | [1,2,4]triazolo[1,5-a]pyrimidine | |
| 331931 | 2-(furan-2-ylmethylamino)benzoate | c1cc(c(cc1)NCc1ccco1)C(═O)O |
| 403374 | (4-chlorophenyl)carbamothioyl- | Clc1ccc(cc1)NC(═S)NC(N)═N |
| | (diaminomethylidene)azanium | |
| 601359 | N-(4-bromonaphthalen-1-yl)-1- | c1cccc2c1c(c(cc2)C(═O)Nc1c2ccccc2c(cc1)Br)O |
| | hydroxynaphthalene-2-carboxamide | |

TABLE 8

| Ligand | Chemical Name | SMILES |
|---|---|---|
| 5069 | 2-[(4S,12S,13R,14S,19R,21S)-9-nitro- | C1C[C]23[CH]4[N]1CC═1[CH](C4)[CH]- |
| | 7,8-dioxo-15-oxa-1,11- | ([CH]3NC═3C2═CC(═O)C(═O)C3N(═O)═O)[CH](OCC1)CC(═O)O |
| | diazahexacyclo[16.3.1.0\hat{ }{4,12}.0\hat{ }{4,21}. | |
| | 0\hat{ }{5,10}.0\hat{ }{13,19}]docosa-5,9,17-trien- | |
| | 14-yl]acetic acid | |
| 21034 | (2Z)-3-(6-nitro-2H-1,3-benzodioxol-5- | O1c2cc(c(cc2OC1)/C═C\C(═O)O)N(═O)═O |
| | yl)prop-2-enoic acid | |
| 25368 | 2-[2-hydroxy-6-(propan-2- | C1CC[CH]([CH]([CH]1OC(C)C)CC(═O)O)O |
| | yloxy)cyclohexyl]acetic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |
| New | | |
| CD81 | | |
| Ligand | Chemical Name | Smiles |
| 5856 | N-[[3-(benzoylcarbamothioylamino)-4- | c1ccccc1C(NC(Nc1cc(c(cc1)C)NC(NC(c1ccccc1)═O)═S)═S)═O |
| | methylphenyl]carbamothioyl]benzamide | |
| 7962 | 3-[4-(2-methylbutan-2- | O(c1ccc(cc1)C(CC)(C)C)c1cccc(c1)C(═O)O |
| | yl)phenoxy]benzoate | |
| 8481 | 2-(4-tert-butylphenoxy)acetate | c1c(OCC(═O)O)ccc(c1)C(C)(C)C |
| 16162 | 2-(ethylamino)benzoate | c1(c(cccc1)NCC)C(═O)O |
| 16646 | 4-propan-2-yloxybenzoate | c1cc(ccc1OC(C)C)C(═O)O |
| 20586 | diaminomethylidene-[4- | c1c(ccc(c1)NC(═N)N)NC(N)═N |
| | (diaminomethylideneazaniumyl)phenyl]azanium | |
| 23895 | 2-carboxy-5-piperidin-1-ylphenolate | N1(CCCCC1)c1cc(c(cc1)C(═O)O)O |
| 25678 | 2-(2,4-dimethylphenyl)quinoline-4- | n1c(c2ccc(cc2C)C)cc(c2c1cccc2)C(═O)O |
| | carboxylate | |
| 31712 | 5-fluoro-2,4-dioxo-1H-pyrimidine-6- | O═c1[nH]c(═O)[nH]c(c1F)C(═O)O |
| | carboxylate | |
| 60239 | 2-(2-prop-2-enylphenoxy)acetate | c1(c(cccc1)CC═C)OCC(═O)O |
| 73170 | (2S)-2-[(3- | Clc1cc(ccc1)NC(═O)O[CH](C)C(═O)O |
| | chlorophenyl)carbamoyloxy]propanoate | |
| 75866 | 2-indol-1-ylacetate | n1(c2ccccc2cc1)CC(═O)O |
| 87504 | 3-[(2R)-piperidin-1-ium-2-yl]pyridine | n1cc(ccc1)[CH]1NCCCC1 |
| 89720 | 3-pyridin-3-ylpropanoate | n1cc(ccc1)CCC(═O)O |
| 94914 | 2-[(2-phenylphenyl)methyl]propanedioate | c1ccccc1c1c(cccc1)CC(C(═O)O)C(═O)O |
| 97538 | butyl-[[(2R)-oxolan-2-yl]methyl]azanium | O1[CH](CCC1)CNCCCC |
| 106863 | 3-oxo-6- | N(/c1ccc(cc1C(═O)O)O)═N\c1ccccc1 |
| | (phenylhydrazinylidene)cyclohexa-1,4- | |
| | diene-1-carboxylate | |
| 117922 | 3-(carbamoylcarbamoyl)pyrazine-2- | n1c(c(ncc1)C(═O)O)C(═O)NC(═O)N |
| | carboxylate | |
| 120631 | 4-(2-phenoxyethoxy)benzoate | O(c1ccc(cc1)C(═O)O)CCOc1ccccc1 |
| 134137 | 3-oxo-4-[(4- | N(═N\c1c(c(cc2c1cccc2)C(═O)O)O)/c1ccc(cc1)S(═O)(═O)N |
| | sulfamoylphenyl)hydrazinylidene]naphthalene- | |
| | 2-carboxylate | |
| 142446 | 4-chloro-N-ethyl-6-(1-phenylindol-3-yl)- | n1(c2ccccc2)c2ccccc2c(c1)c1nc(Cl)nc(n1)NCC |
| | 1,3,5-triazin-2-amine | |
| 144958 | (Z)-4-oxo-4-(quinolin-8-ylamino)but-2- | n1cccc2c1c(ccc2)NC(/C═C\C(═O)O)═O |
| | enoate | |
| 148832 | (2E,4E)-5-[(1R)-1-hydroxy-2,6,6- | O═C1CC([C](C(═C1)C)(/C═C/C(C)═C/C(═O)O)O)(C)C |
| | trimethyl-4-oxocyclohex-2-en-1-yl]-3- | |
| | methylpenta-2,4-dienoate | |
| 153172 | 2-[2-(3-methoxy-4-oxocyclohexa-2,5- | N(/c1cc(c(cc1)O)OC)═N\c1ccccc1C(═O)O |
| | dien-1-ylidene)hydrazinyl]benzoate | |
| 215276 | 4-(2,5-dimethylphenyl)-4-oxobutanoate | c1(c(cc(cc1)C)C(═O)CCC(═O)O)C |
| 252359 | 2-(4-chlorophenyl)-5-methyl-7-(4- | c1(nc2n(c(c1)N1CCN(CC1)C)nc(n2)c1ccc(cc1)Cl)C |
| | methylpiperazin-4-ium-1-yl)- | |
| | [1,2,4]triazolo[1,5-a]pyrimidine | |
| 331931 | 2-(furan-2-ylmethylamino)benzoate | c1cc(c(cc1)NCc1cccc1)C(═O)O |
| 403374 | (4-chlorophenyl)carbamothioyl- | Clc1ccc(cc1)NC(═S)NC(N)═N |
| | (diaminomethylidene)azanium | |
| 601359 | N-(4-bromonaphthalen-1-yl)-1- | c1cccc2c1c(c(cc2)C(═O)Nc1c2ccccc2c(cc1)Br)O |
| | hydroxynaphthalene-2-carboxamide | |

TABLE 9

| Ligand | Chemical Name | SMILES |
|---|---|---|
| 11891 | 2-(1,3-benzothiazol-2-ylsulfanyl)acetic | c1cccc2c1nc(SCC(═O)O)s2 |
| | acid | |
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 41066 | 2-[(2-nitrophenyl)carbamoyl]benzoic acid | c1cccc(c1C(Nc1ccccc1N(═O)═O)═O)C(═O)O |
| 55573 | (2E)-3-[(2-methylpropyl)carbamoyl]prop- | C(═C\C(═O)NCC(C)C)/C(═O)O |
| | 2-enoic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |

Table 9 ligands also include 5856, 8481, 20586, 25678, 60239, 75866, 87504, 89720, 142446, 148832, 2i5276, 252359, 331931, 403374, and 601359, which are described in Table 3.

TABLE 10

| Ligand | Chemical Name | SMILES |
|---|---|---|
| 21034 | (2Z)-3-(6-nitro-2H-1,3-benzodioxol-5- | O1c2cc(c(cc2OC1)/C═C\C(═O)O)N(═O)═O |
| | yl)prop-2-enoic acid | |
| 25368 | 2-[2-hydroxy-6-(propan-2- | C1CC[CH]([CH]([CH]1OC(C)C)CC(═O)O)O |
| | yloxy)cyclohexyl]acetic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 10 ligands also include 5856, 7962, 848i, 16162, 16646, 20586, 23895, 25678, 31712, 60239, 73170, 75866, 87504, 89720, 94914, 97538, 106863, 117922, 120631, 134137, 142446, 144958, 148832, 153172, 2i5276, 252359, 331,931, 403374, and 601359 which are described above in Table 8.

TABLE 11

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 41066 | 2-[(2-nitrophenyl)carbamoyl]benzoic acid | c1cccc(c1C(Nc1ccccc1N(═O)═O)═O)C(═O)O |
| 55573 | (2E)-3-[(2-methylpropyl)carbamoyl]prop- | C(═C\C(═O)NCC(C)C)/C(═O)O |
| | 2-enoic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |

Table 11 ligands also include 5856, 8481, 20586, 25678, 60239, 75866, 87504, 89720, 142446, 148832, 215276, 331931, 403374, and 601359, which are described in Table 3.

TABLE 12

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 12 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 13

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 68971 | 2-hydroxy-5- | c1ccccc1OC(Nc1cc(c(cc1)O)C(═O)O)═O |
| | [(phenoxycarbonyl)amino]benzoic acid | |
| 68982 | 1-[(E)-[(2,6- | Clc1c(c(Cl)ccc1)/C═N/NC(N)═N |
| | dichlorophenyl)methylidene]amino]guanidine | |
| 70980 | 2-(2-amino-4-hydroxy-1,3-thiazol-5- | c1(nc(sc1CC(═O)O)N)O |
| | yl)acetic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 13 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 14

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 68982 | 1-[(E)-[(2,6- | Clc1c(c(Cl)ccc1)/C═N/NC(N)═N |
| | dichlorophenyl)methylidene]amino]guanidine | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 408860 | 2-[(3-nitrophenyl)carbamoyl]benzoic acid | c1cccc(c1C(═O)O)C(═O)Nc1cc(ccc1)N(═O)═O |

Table 14 ligands also include 31712, 73170, 94914, 97538, 106863, 117922, 120631, 134137, 144958, 153172, and 252359, which are described above in Table 6. Table 14 ligands also include 5856, 7962, 8481, 16646, 20586, 23895, 75866, 87504, 142446, 148832, 252359, 331931, 403374, 601359 which are described above in Table 7.

TABLE 15

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 70980 | 2-(2-amino-4-hydroxy-1,3-thiazol-5- | c1(nc(sc1CC(═O)O)N)O |
| | yl)acetic acid | |
| 73735 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |
| New | | |
| CD81 | | |
| Ligand | Chemical Name | Smiles |
| 5856 | N-[[3-(benzoylcarbamothioylamino)-4- | c1ccccc1C(NC(Nc1cc(c(cc1)C)NC(NC(c1ccccc1)═O)═S)═S)═O |
| | methylphenyl]carbamothioyl]benzamide | |
| 8481 | 2-(4-tert-butylphenoxy)acetate | c1c(OCC(═O)O)ccc(c1)C(C)(C)C |
| 16162 | 2-(ethylamino)benzoate | c1(c(cccc1)NCC)C(═O)O |
| 20586 | diaminomethylidene-[4- | c1c(ccc(c1)NC(═N)N)NC(N)═N |
| | (diaminomethylideneazaniumyl)phenyl]azanium | |
| 23895 | 2-carboxy-5-piperidin-1-ylphenolate | N1(CCCCC1)c1cc(c(cc1)C(═O)O)O |
| 25678 | 2-(2,4-dimethylphenyl)quinoline-4- | n1c(c2ccc(cc2C)C)cc(c2c1cccc2)C(═O)O |
| | carboxylate | |
| 60239 | 2-(2-prop-2-enylphenoxy)acetate | c1(c(cccc1)CC═C)OCC(═O)O |
| 75866 | 2-indol-1-ylacetate | n1(c2ccccc2cc1)CC(═O)O |
| 87504 | 3-[(2R)-piperidin-1-ium-2-yl]pyridine | n1cc(ccc1)[CH]1NCCCC1 |
| 89720 | 3-pyridin-3-ylpropanoate | n1cc(ccc1)CCC(═O)O |
| 215276 | 4-(2,5-dimethylphenyl)-4-oxobutanoate | c1(c(cc(cc1)C)C(═O)CCC(═O)O)C |
| 252359 | 2-(4-chlorophenyl)-5-methyl-7-(4- | c1(nc2n(c(c1)N1CCN(CC1)C)nc(n2)c1ccc(cc1)Cl)C |
| | methylpiperazin-4-ium-1-yl)- | |
| | [1,2,4]triazolo[1,5-a]pyrimidine | |
| 331931 | 2-(furan-2-ylmethylamino)benzoate | c1cc(c(cc1)NCc1ccco1)C(═O)O |
| 403374 | (4-chlorophenyl)carbamothioyl- | Clc1ccc(cc1)NC(═S)NC(N)═N |
| | (diaminomethylidene)azanium | |

Table 15 ligands also include 7962, 16646, 31712, 73170, 94914, 97538, 106863, 117922, 120631, 134137, 142446, 144958, 148832, 153172, 164965, 165665, 215359, and 601359 which are described above in Table 6.

TABLE 16

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 70980 | 2-(2-amino-4-hydroxy-1,3-thiazol-5- | c1(nc(sc1CC(═O)O)N)O |
| | yl)acetic acid | |
| 75846 | 4-(methylamino)piperidine-4- | N1CCC(CC1)(NC)C(═O)N |
| | carboxamide | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 16 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 17

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 68971 | 2-hydroxy-5- | c1ccccc1OC(Nc1cc(c(cc1)O)C(═O)O)═O |
| | [(phenoxycarbonyl)amino]benzoic acid | |
| 70980 | 2-(2-amino-4-hydroxy-1,3-thiazol-5- | c1(nc(sc1CC(═O)O)N)O |
| | yl)acetic acid | |
| 78623 | 4-carbamoyl-2-(5-hydroxy-1,3-dioxo-2,3- | O═C1N(C(═O)c2c1ccc(c2)O)[CH](C(═O)O)CCC(═O)N |
| | dihydro-1H-isoindol-2-yl)butanoic acid | |
| 81750 | 4-{[(1,3-dimethyl-2,6-dioxo-2,3,6,7- | O═c1n(c(═O)c2[nH]c(nc2n1C)CNc1ccc(cc1)C(═O)O)C |
| | tetrahydro-1H-purin-8- | |
| | yl)methyl]amino}benzoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 17 ligands also include 31712, 73170, 94914, 97538, 106863, 117922, 120631, 134137, 144958, 153172, and 252359, which are described above in Table 6. Table 17 ligands also include 5856, 7962, 8481, 16646, 20586, 23895, 75866, 87504, 142446, 148832, 252359, 331931, 403374, 601359 which are described above in Table 7.

TABLE 18

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |

Table 18 ligands also include 31712, 73170, 94914, 97538, 106863, 117922, 120631, 134137, 144958, 153172, and 252359, which are described above in Table 6. Table 18 ligands also include 5856, 7962, 8481, 16646, 20586, 23895, 75866, 87504, 142446, 148832, 252359, 331931, 403374, 601359 which are described above in Table 7.

TABLE 19

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 98026 | 2-{[3-(3-methylbut-2-en-1-yl)-1,4-dioxo- | O═C1C(═C(OCC(═O)O)C(═O)c2c1cccc2)C/C═C(\C)C |
| | 1,4-dihydronaphthalen-2-yl]oxy}acetic | |
| | acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |

Table 19 ligands also include 31712, 73170, 94914, 97538, 106863, 117922, 120631, 134137, 144958, 153172, and 252359, which are described above in Table 6. Table 19 ligands also include 5856, 7962, 8481, 16646, 20586, 23895, 75866, 87504, 142446, 148832, 252359, 331931, 403374, 601359 which are described above in Table 7.

TABLE 20

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)CCC(═O)O |
| | yl)propanoic acid | |
| 70980 | 2-(2-amino-4-hydroxy-1,3-thiazol-5- | c1(nc(sc1CC(═O)O)N)O |
| | yl)acetic acid | |
| 127947 | 2-[(4-{4-[(2-carboxyphenyl)amino]-3- | c1c(ccc(c1OC)Nc1ccccc1C(═O)O)c1cc(c(cc1)Nc1ccccc1C(═O)O)OC |
| | methoxyphenyl}-2- | |
| | methoxyphenyl)amino]benzoic acid | |
| 156957 | 1-[(benzyloxy)carbonyl]-5- | N1([CH](CCC1═O)C(═O)O)C(OCc1ccccc1)═O |
| | oxopyrrolidine-2-carboxylic acid | |
| 303800 | 3-(3-methoxy-1,2-oxazol-5-yl)propanoic | c1(onc(c1)OC)CCC(═O)O |
| | acid | |
| 362639 | 2-[2-(3,5-dioxo-2,3,4,5-tetrahydro-1,2,4- | c1cc(c2c(═O)[nH]c([nH]n2)═O)c(cc1)n1nc(c([nH]c1═O)═O)C(═O)O |
| | triazin-6-yl)phenyl]-3,5-dioxo-2,3,4,5- | |
| | tetrahydro-1,2,4-triazine-6-carboxylic | |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 408860 | 2-[(3-nitrophenyl)carbamoyl]benzoic acid | c1cccc(c1C(═O)O)C(═O)Nc1cc(ccc1)N(═O)═O |
| 638134 | (2E)-2-cyano-3-(2-methoxyphenyl)prop- | c1cc(c(cc1)/C═C(/C(═O)O)C#N)OC |
| | 2-enoic acid | |

Table 20 ligands also include 5856, 8481, 20586, 25678, 60239, 75866, 87504, 89720, 142446, 148832, 215276, 331931, 403374, and 601359, which are described in Table 3.

TABLE 21

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 70980 | 2-(2-amino-4-hydroxy-1,3-thiazol-5- | c1(nc(sc1CC(═O)O)N)O |
| | yl)acetic acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 21 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 22

| Ligand | Chemical Name | SMILES |
|---|---|---|
| 25368 | 2-[2-hydroxy-6-(propan-2- | C1CC[CH]([CH]([CH]1OC(C)C)CC(═O)O)O |
| | yloxy)cyclohexyl]acetic acid | |
| 70980 | 2-(2-amino-4-hydroxy-1,3-thiazol-5- | c1(nc(sc1CC(═O)O)N)O |
| | yl)acetic acid | |
| 123115 | [7-methoxy-12-methyl-10,13-dioxo-11- | O═C1C2═C(N3[C]([CH]2COC(═O)N)([CH]2[CH](C3)N2)OC)C(═O)C(═C1NCCC)C |
| | (propylamino)-2,5- | |
| | diazatetracyclo[7.4.0.0\hat{ }{2,7}.0\hat{ }{4,6}]trideca- | |
| | 1(9),11-dien-8-yl]methyl carbamate | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C(═O)O)Cc1c2c([nH]c1)cccc2 |
| | yl)-3-(1H-indol-3-yl)propanoic acid | |
| 408734 | 4-[(Z)-2-(triaminopyrimidin-5-yl)diazen- | n1c(c(/N═N\c2ccc(cc2)C(═O)O)c(nc1N)N)N |
| | 1-yl]benzoic acid | |
| 638134 | (2E)-2-cyano-3-(2-methoxyphenyl)prop- | c1cc(c(cc1)/C═C(/C(═O)O)C#N)OC |
| | 2-enoic acid | |

Table 22 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 23

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)C |
| | yl)propanoic acid | CC(═O)O |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 362639 | 2-[2-(3,5-dioxo-2,3,4,5-tetrahydro-1,2,4- | c1cc(c2c(═O)[nH]c([nH]n2)═O)c |
| | triazin-6-yl)phenyl]-3,5-dioxo-2,3,4,5- | (cc1)n1nc(c([nH]c1═O)═O)C(═O) |
| | tetrahydro-1,2,4-triazine-6-carboxylic | O |
| | acid | |
| 408734 | 4-[(Z)-2-(triaminopyrimidin-5-yl)diazen- | n1c(c(/N═N\c2ccc(cc2)C(═O)O)c |
| | 1-yl]benzoic acid | (nc1N)N)N |

Table 23 ligands also include 31712, 73170, 94914, 97538, 106863, 117922, 120631, 134137, 144958, 153172, and 252359, which are described above in Table 6. Table 23 ligands also include 5856, 7962, 8481, 16646, 20586, 23895, 75866, 87504, 142446, 148832, 252359, 331931, 403374, 601359 which are described above in Table 7.

TABLE 24

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)C |
| | yl)propanoic acid | CC(═O)O |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 362639 | 2-[2-(3,5-dioxo-2,3,4,5-tetrahydro-1,2,4- | c1cc(c2c(═O)[nH]c([nH]n2)═O)c |
| | triazin-6-yl)phenyl]-3,5-dioxo-2,3,4,5- | (cc1)n1nc(c([nH]c1═O)═O)C(═O) |
| | tetrahydro-1,2,4-triazine-6-carboxylic | O |
| | acid | |
| 408734 | 4-[(Z)-2-(triaminopyrimidin-5-yl)diazen- | n1c(c(/N═N\c2ccc(cc2)C(═O)O)c |
| | 1-yl]benzoic acid | (nc1N)N)N |

Table 24 ligands also include 31712, 73170, 94914, 97538, 106863, 117922, 120631, 134137, 144958, 153172, and 252359, which are described above in Table 6. Table 24 ligands also include 5856, 7962, 8481, 16646, 20586, 23895, 75866, 87504, 142446, 148832, 252359, 331931, 403374, 601359 which are described above in Table 7.

TABLE 25

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)C |
| | yl)propanoic acid | CC(═O)O |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 362639 | 2-[2-(3,5-dioxo-2,3,4,5-tetrahydro-1,2,4- | c1cc(c2c(═O)[nH]c([nH]n2)═O)c |
| | triazin-6-yl)phenyl]-3,5-dioxo-2,3,4,5- | (cc1)n1nc(c([nH]c1═O)═O)C(═O) |
| | tetrahydro-1,2,4-triazine-6-carboxylic | O |
| | acid | |
| 408734 | 4-[(Z)-2-(triaminopyrimidin-5-yl)diazen- | n1c(c(/N═N\c2ccc(cc2)C(═O)O)c |
| | 1-yl]benzoic acid | (nc1N)N)N |

Table 25 ligands also include 31712, 73170, 94914, 97538, 106863, 117922, 120631, 134137, 144958, 153172, and 252359, which are described above in Table 6. Table 25 ligands also include 5856, 7962, 8481, 16646, 20586, 23895, 75866, 87504, 142446, 148832, 252359, 331931, 403374, 601359 which are described above in Table 7.

TABLE 26

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 70980 | 2-(2-amino-4-hydroxy-1,3-thiazol-5- | c1(nc(sc1CC(═O)O)N)O |
| | yl)acetic acid | |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 638134 | (2E)-2-cyano-3-(2-methoxyphenyl)prop- | c1cc(c(cc1)/C═C(/C(═O)O)C#N) |
| | 2-enoic acid | OC |

Table 26 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 27

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 70980 | 2-(2-amino-4-hydroxy-1,3-thiazol-5- | c1(nc(sc1CC(═O)O)N)O |
| | yl)acetic acid | |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 638134 | (2E)-2-cyano-3-(2-methoxyphenyl)prop- | c1cc(c(cc1)/C═C(/C(═O)O)C#N) |
| | 2-enoic acid | OC |

Table 27 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 28

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 70980 | 2-(2-amino-4-hydroxy-1,3-thiazol-5- | c1(nc(sc1CC(═O)O)N)O |
| | yl)acetic acid | |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 638134 | (2E)-2-cyano-3-(2-methoxyphenyl)prop- | c1cc(c(cc1)/C═C(/C(═O)O)C#N) |
| | 2-enoic acid | OC |

Table 28 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 29

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 36914 | 2-(6-sulfanylidene-6,9-dihydro-3H-purin- | n1c[nH]c2n(cnc2c1═S)CC(═O)O |
| | 9-yl)acetic acid | |
| 70980 | 2-(2-amino-4-hydroxy-1,3-thiazol-5- | c1(nc(sc1CC(═O)O)N)O |
| | yl)acetic acid | |
| 638134 | (2E)-2-cyano-3-(2-methoxyphenyl)prop- | c1cc(c(cc1)/C═C(/C(═O)C#N) |
| | 2-enoic acid | OC |

Table 29 ligands also include 5856, 8481, 20586, 25678, 60239, 75866, 87504, 89720, 142446, 148832, 215276, 331931, 403374, and 601359, which are described in Table 3.

TABLE 30

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 36914 | 2-(6-sulfanylidene-6,9-dihydro-3H-purin- | n1c[nH]c2n(cnc2c1═S)CC(═O)O |
| | 9-yl)acetic acid | |

Table 30 ligands also include 31712, 73170, 94914, 97538, 106863, 117922, 120631, 134137, 144958, 153172, and 252359, which are described above in Table 6. Table 30 ligands also include 5856, 7962, 8481, 16646, 20586, 23895, 75866, 87504, 142446, 148832, 252359, 331931, 403374, 601359 which are described above in Table 7.

TABLE 31

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 638134 | (2E)-2-cyano-3-(2-methoxyphenyl)prop- | c1cc(c(cc1)/C═C(/C(═O)O)C#N) |
| | 2-enoic acid | OC |

Table 31 ligands also include 5856, 7962, 8481, 16162, 16646, 20586, 23895, 25678, 31712, 60239, 73170, 75866, 87504, 89720, 94914, 97538, 106863, 117922, 120631, 134137, 142446, 144958, 148832, 153172, 215276, 252359, 331,931, 403374, and 601359 which are described above in Table 8.

TABLE 32

| Ligand | Chemical Name | SMILES |
|---|---|---|
| 16631 | 3,4-dihydroxybenzoic acid | c1(cc(ccc1O)C(═O)O)O |
| 68971 | 2-hydroxy-5- | c1ccccc1OC(Nc1cc(c(cc1)O)C |
| | [(phenoxycarbonyl)amino]benzoic acid | (═O)O)═O |
| 78623 | 4-carbamoyl-2-(5-hydroxy-1,3-dioxo-2,3- | O═C1N(C(═O)c2c1ccc(c2)O) |
| | dihydro-1H-isoindol-2-yl)butanoic acid | [CH](C(═O)O)CCC(═O)N |
| 81750 | 4-{[(1,3-dimethyl-2,6-dioxo-2,3,6,7- | O═c1n(c(═O)c2[nH]c(nc2n1C)CN |
| | tetrahydro-1H-purin-8- | c1ccc(cc1)C(═O)O)C |
| | yl)methyl]amino}benzoic acid | |
| 303800 | 3-(3-methoxy-1,2-oxazol-5-yl)propanoic | c1(onc(c1)OC)CCC(═O)O |
| | acid | |
| 401077 | 2-(1,3-dioxo-2,3-dihydro-1H-isoindol-2- | O═C1N(C(═O)c2c1cccc2)[CH](C |
| | yl)-3-(1H-indol-3-yl)propanoic acid | (═O)O)Cc1c2c([nH]c1)cccc2 |
| 408734 | 4-[(Z)-2-(triaminopyrimidin-5-yl)diazen- | n1c(c(/N═N\c2ccc(cc2)C(═O)O)c |
| | 1-yl]benzoic acid | (nc1N)N)N |

TABLE 33

| Ligand | Chemical Name | SMILES |
|---|---|---|
| 5069 | 2-[(4S,12S,13R,14S,19R,21S)-9-nitro- | C1C[C]23[CH]4[N]1CC═1[CH] |
| | 7,8-dioxo-15-oxa-1,11- | (C4)[CH]([CH]3NC═3C2═CC(═O) |
| | diazahexacyclo[16.3.1.0\hat{ }{4,12}.0\hat{ }{4,21} | C(═O)C3N(═O)═O)[CH](OCC1)C |
| | .0\hat{ }{5,10}.0\hat{ }{13,19}]docosa-5,9,17-trien- | C(═O)O |
| | 14-yl]acetic acid | |
| 7436 | 4-amino-N-(4-tert-butylphenyl)benzene- | c1cc(ccc1NS(c1ccc(cc1)N) |
| | 1-sulfonamide | (═O)═O)C(C)(C)C |
| 21034 | (2Z)-3-(6-nitro-2H-1,3-benzodioxol-5- | O1c2cc(c(cc2OC1)/C═C\C(═O)O) |
| | yl)prop-2-enoic acid | N(═O)═O |
| 30930 | 5-(benzyloxy)-1H-indole-2-carboxylic | [nH]1c2ccc(cc2cc1C(═O)O)OCc1 |
| | acid | ccccc1 |
| 98026 | 2-{[3-(3-methylbut-2-en-1-yl)-1,4-dioxo- | O═C1C(═C(OCC(═O)O)C(═O)c2 |
| | 1,4-dihydronaphthalen-2-yl]oxy}acetic | c1cccc2)C/C═C(\C)C |
| | acid | |
| 123115 | [7-methoxy-12-methyl-10,13-dioxo-11- | O═C1C2═C(N3[C]([CH]2COC |
| | (propylamino)-2,5-diazatetra- | (═O)N)([CH]2[CH](C3)N2)OC)C |
| | cyclo[7.4.0.0\hat{ }{2,7}.0\hat{ }{4,6}]trideca- | (═O)C(═C1NCCC)C |
| | 1(9),11-dien-8-yl]methyl carbamate | |
| 165665 | (3-hydroxyphenyl)thiourea | c1ccc(cc1NC(═S)N)O |
| 689002 | 9-Oxo-9H-thioxanthene-3-carboxamide | c1ccc2c(c1)C(c1c(S2(═O)═O)cc |
| | 10,10-dioxide | (cc1)C(═O)N)═O |

TABLE 34

| Ligand | Chemical Name | SMILES |
|---|---|---|
| 11891 | 2-(1,3-benzothiazol-2-ylsulfanyl)acetic | c1cccc2c1nc(SCC(═O)O)s2 |
| | acid | |
| 36914 | 2-(6-sulfanylidene-6,9-dihydro-3H-purin- | n1c[nH]c2n(cnc2c1═S)CC(═O)O |
| | 9-yl)acetic acid | |
| 38743 | 3-(2,5-dioxo-4-phenylimidazolidin-4- | O═C1N[C](c2ccccc2)(C(═O)N1)C |
| | yl)propanoic acid | CC(═O)O |
| 41066 | 2-[(2-nitrophenyl)carbamoyl]benzoic acid | c1cccc(c1C(Nc1ccccc1N(═O)═O)═O) |
| | | C(═O)O |
| 55573 | (2E)-3-[(2-methylpropyl)carbamoyl]prop- | C(═C\C(═O)NCC(C)C)/C(═O)O |
| | 2-enoic acid | |
| 63865 | 5-{2-oxo-hexahydro-1H-thieno[3,4- | O═C1N[CH]2[CH](N1)[CH] |
| | d]imidazolidin-4-yl}pentanoic acid | (SC2)CCCCC(═O)O |
| 73735 | 2-[(4-{4-[(2-carboxyphenyl)amino]-3- | c1c(ccc(c1OC)Nc1ccccc1C(═O)O) |
| | methoxyphenyl}-2- | c1cc(c(cc1)Nc1ccccc1C(═O)O)O |
| | methoxyphenyl)amino]benzoic acid | C |
| 127947 | 2-[(4-{4-[(2-carboxyphenyl)amino]-3- | c1c(ccc(c1OC)Nc1ccccc1C(═O)O) |
| | methoxyphenyl}-2- | c1cc(c(cc1)Nc1ccccc1C(═O)O)O |
| | methoxyphenyl)amino]benzoic acid | C |
| 156957 | 1-[(benzyloxy)carbonyl]-5- | N1([CH](CCC1═O)C(═ O)O)C |
| | oxopyrrolidine-2-carboxylic acid | (OCc1ccccc1)═O |
| 362639 | 2-[2-(3,5-dioxo-2,3,4,5-tetrahydro-1,2,4- | c1cc(c2c(═O)[nH]c([nH]n2)═O)c |
| | triazin-6-yl)phenyl]-3,5-dioxo-2,3,4,5- | (cc1)n1nc(c([nH]c1═O)═O)C(═O) |
| | tetrahydro-1,2,4-triazine-6-carboxylic | O |
| | acid | |

TABLE 35

| Ligand | Chemical Name | SMILES |
|---|---|---|
| 25368 | 2-[2-hydroxy-6-(propan-2- | C1CC[CH]([CH]([CH]1OC(C)C) |
| | yloxy)cyclohexyl]acetic acid | CC(═O)O)O |
| 93033 | 4-[2-(2,4-dioxo-1,2,3,4- | O═c1n(ccc([nH]1)═O)CC(Nc1cc |
| | tetrahydropyrimidin-1-yl)acetamido]-2- | (c(cc1)C(═O)O)O)═O |
| | hydroxybenzoic acid | |

TABLE 36

| Ligand | Chemical Name | SMILES |
|---|---|---|
| 68982 | 1-[(E)-[(2,6- | Clc1c(c(Cl)ccc1)/C═N/NC(N)═N |
| | dichlorophenyl)methylidene]amino]gua- | |
| | nidine | |

TABLE 37

| Ligand | Chemical Name | SMILES |
|---|---|---|
| 75846 | 4-(methylamino)piperidine-4- | N1CCC(CC1)(NC)C(═O)N |
| | carboxamide | |
| 408860 | 2-[(3-nitrophenyl)carbamoyl]benzoic acid | c1cccc(c1C(═O)O)C(═O)Nc1cc |
| | | (ccc1)N(═O)═O |

### Representative HCV E2 and Human CD81 Amino Acid Sequences

The HCV E2 amino acid sequence described by C4MR37 [UniParc] available at http://www.uniprot.org/uniprot/C4MR37 is described by SEQ ID NO: 1. Web Link: http://www.uniprot.org/uniprot/C4MR37. C4MR37 [UniParc]. This sequence contains residues 384-746 of a longer 3011 amino acid sequence. The human CD81 Sequence described by NCBI Reference Sequence NP_{—}004347.1 (gi|4757944|ref|NP_{—}004347.1|CD81 antigen [Homo sapiens]) appears in SEQ ID NO: 2. The Chain A crystal structure of CD81 extracellular domain, which is a receptor for HCV, is available as Accession number 1G8Q_A (version 1G8Q_A GI:13399775). The corresponding amino acid sequence is given by SEQ ID NO: 3. The Chain B crystal strcture of CD81 extracellular domain, which is a receptor for HCV, is available as Accession number 1G8Q_B (version 1G8Q_B GI:13399776). The corresponding amino acid sequence is given by SEQ ID NO: 4.

### REFERENCES

1. Ferrari, C.; Urbani, S.; Penna, A.; Cavalli, A.; Valli, A.; Lamonaca, V.; Bertoni, R.; Boni, C.; Barbieri, K.; Uggeri, J.; Fiaccadori, F., Immunopathogenesis of hepatitis C virus infection. J Hepat 1999, 31, (Supplement 1), 3-8.
2. Bartenschlager, R., The NS3/4A proteinase of the hepatitis C virus: unravelling structure and function of an unusual enzyme and a prime target for antiviral therapy. J Viral Hepat 1999, 6, (3), 165-81.
3. Lesburg, C. A.; Radfar, R.; Weber, P. C., Recent advances in the analysis of HCV NS5B RNA-dependent RNA polymerase. Curr Opin Investig Drugs 2000, 1, (3), 289-96.
4. Welbourn, S.; Pause, A., The hepatitis C virus NS2/3 protease. Curr Issues Mol Biol 2007, 9, (1), 63-9.
5. Venkatraman, S.; Njoroge, F. G., Macrocyclic inhibitors of HCV NS3 protease. Expert Opin Ther Pat 2009, 19, (9), 1277-303.
6. Enomoto, M.; Tamori, A.; Kawada, N., Emerging antiviral drugs for hepatitis C virus. Rev Recent Clin Trials 2009, 4, (3), 179-84.
7. Chary, A.; Holodniy, M., Recent advances in hepatitis C virus treatment: review of HCV protease inhibitor clinical trials. Rev Recent Clin Trials 2010, 5, (3), 158-73.
8. Sharma, S. D., Hepatitis C virus: molecular biology & current therapeutic options. Indian J Med Res 2010, 131, 17-34.
9. Stoll-Keller, F.; Barth, H.; Fafi-Kremer, S.; Zeisel, M. B.; Baumert, T. F., Development of hepatitis C virus vaccines: challenges and progress. Expert Rev Vaccines 2009, 8, (3), 333-45.
10. Dubuisson, J., Hepatitis C virus proteins. World J Gastroenterol 2007, 13, (17), 2406-15.
11. Budkowska, A., Mechanism of cell infection with hepatitis C virus (HCV)—a new paradigm in virus-cell interaction. Pol J Microbiol 2009, 58, (2), 93-8.
12. Bartosch, B.; Vitelli, A.; Granier, C.; Goujon, C.; Dubuisson, J.; Pascale, S.; Scarselli, E.; Cortese, R.; Nicosia, A.; Cosset, F. L., Cell entry of hepatitis C virus requires a set of co-receptors that include the CD81 tetraspanin and the SR-B1 scavenger receptor. J Biol Chem 2003, 278, (43), 41624-30.
13. Bartosch, B.; Cosset, F. L., Cell entry of hepatitis C virus. Virology 2006, 348, (1), 1-12.
14. Pileri, P.; Uematsu, Y.; Campagnoli, S.; Galli, G.; Falugi, F.; Petracca, R.; Weiner, A. J.;
Houghton, M.; Rosa, D.; Grandi, G.; Abrignani, S., Binding of hepatitis C virus to CD81. Science 1998, 282, (5390), 938-41.
15. Levy, S.; Todd, S. C.; Maecker, H. T., CD81 (TAPA-1): a molecule involved in signal transduction and cell adhesion in the immune system. Annu Rev Immunol 1998, 16, 89-109.
16. Petracca, R.; Falugi, F.; Galli, G.; Norais, N.; Rosa, D.; Campagnoli, S.; Burgio, V.; Di Stasio, E.; Giardina, B.; Houghton, M.; Abrignani, S.; Grandi, G., Structure-function analysis of hepatitis C virus envelope-CD81 binding. J Virol 2000, 74, (10), 4824-30.
17. Higginbottom, A.; Quinn, E. R.; Kuo, C. C.; Flint, M.; Wilson, L. H.; Bianchi, E.; Nicosia, A.; Monk, P. N.; McKeating, J. A.; Levy, S., Identification of amino acid residues in CD81 critical for interaction with hepatitis C virus envelope glycoprotein E2. J Virol 2000, 74, (8), 3642-9.
18. Zhang, Y. Y.; Zhang, B. H.; Ishii, K.; Liang, T. J., Novel function of CD81 in controlling hepatitis C virus replication. J Virol 2010, 84, (7), 3396-407.
19. Drummer, H. E.; Wilson, K. A.; Poumbourios, P., Identification of the hepatitis C virus E2 glycoprotein binding site on the large extracellular loop of CD81. J Virol 2002, 76, (21), 11143-7.
20. VanCompernolle, S. E.; Wiznycia, A. V.; Rush, J. R.; Dhanasekaran, M.; Baures, P. W.; Todd, S. C., Small molecule inhibition of hepatitis C virus E2 binding to CD81. Virology 2003, 314, (1), 371-80.
21. Kitadokoro, K.; Bordo, D.; Galli, G.; Petracca, R.; Falugi, F.; Abrignani, S.; Grandi, G.; Bolognesi, M., CD81 extracellular domain 3D structure: insight into the tetraspanin superfamily structural motifs. Embo J2001, 20, (1-2), 12-8.
22. Kitadokoro, K.; Galli, G.; Petracca, R.; Falugi, F.; Grandi, G.; Bolognesi, M., Crystallization and preliminary crystallographic studies on the large extracellular domain of human CD81, a tetraspanin receptor for hepatitis C virus. Acta Crystallogr D Biol Crystallogr 2001, 57, (Pt 1), 156-8.
23. Balhorn, R.; Hok, S.; Burke, P. A.; Lightstone, F. C.; Cosman, M.; Zemla, A.; Mirick, G.; Perkins, J.; Natarajan, A.; Corzett, M.; DeNardo, S. J.; Albrecht, H.; Gregg, J. P.; DeNardo, G. L., Selective high-affinity ligand antibody mimics for cancer diagnosis and therapy: initial application to lymphoma/leukemia. Clin Cancer Res 2007, 13, (18 Pt 2), 5621s-5628s.
24. DeNardo, G. L.; Hok, S.; Van Natarajan, A.; Cosman, M.; DeNardo, S. J.; Lightstone, F. C.; Mirick, G. R.; Yuan, A.; Perkins, J.; Sysko, V. V.; Lehmann, J.; Balhorn, R. L., Characteristics of dimeric (bis) bidentate selective high affinity ligands as HLA-DR10 beta antibody mimics targeting non-Hodgkin's lymphoma. Int J Oncol 2007, 31, (4), 729-40.
25. AutoDock website: http://autodock.scripps.edu
26. Morris, G. M.; Goodsell D. S.; Halliday R. S.; Huey R.; Hart W. E.; Belew R. K.; Olson A. K., Automated docking using a Lamarckian genetic algorithm and an empirical binding free energy function. J. Comput. Chem, 1998, 19, 1639-1662.
27. Huey, R.; Morris, G. M.; Olson, A. J.; Goodsell, D. S., A semiempirical free energy force field with charge-based desolvation. J Comput Chem 2007, 28, (6), 1145-52.
28. Huey R.; Goodsell D. S.; Morris G. M.; Olson A. J., Grid-based hydrogen bond potentials with improved directionality. Letters in Drug Design and Discovery 2004, 1, 178-183.
29. Harris, R.; Olson, A. J.; Goodsell, D. S., Automated prediction of ligand-binding sites in proteins. Proteins 2008, 70, (4), 1506-17.
30. Morris, G. M., Huey, R., Olson, A., Using AutoDock for Ligand-Receptor Docking. Current Protocols in Bioinformatics Chapter 8 2008, Unit 8.14.
31. NBCR website: https://www.nbcr.net/pub/wiki/index.php?title=CADD Pipeline
32. Sanner, M. F., Python: a programming language for software integration and development. J Mol Graph Model 1999, 17, (1), 57-61.
33. Holzer, M.; Ziegler, S.; Neugebauer, A.; Kronenberger, B.; Klein, C. D.; Hartmann, R. W., Structural modifications of salicylates: inhibitors of human CD81-receptor HCV-E2 interaction. Arch Pharm (Weinheim) 2008, 341, (8), 478-84.

### INCORPORATION BY REFERENCE

Each document, patent, patent application or patent publication cited by or referred to in this disclosure is incorporated by reference in its entirety, especially with respect to the specific subject matter surrounding the citation of the reference in the text. For example, the amino acid sequences, structural features, and crystal structures of CD81 and HCV E2 are incorporated by reference to the publications or data bank entries (e.g., Protein Data Bank Entry 1G8Q) above describing those structures. Similarly, the Autodock suite of programs and tools (ADTs, including AutoLigand) are incorporated by reference to the publications or other resources cited herein that describe them. No admission is made that any such reference constitutes background art and the right to challenge the accuracy and pertinence of the cited documents is reserved.

1. A ligand conjugate that comprises at least two ligands that bind to at least one of Sites 1, 2, 3, 4, or 5 on CD81 or that inhibits the binding of a molecule known to bind to at least one of Sites 1, 2, 3, 4, or 5 to the site.

2. The ligand conjugate of claim 1 that comprises at least one ligand selected from the group consisting of 5069, 7436, 7962, 16646, 21034, 23895, 30930, 31712, 73170, 94914, 97538, 98026, 106963, 117922, 120631, 123115, 134137, 144958, 153172, 164965, 165665, 252359, and 689002.

3. The ligand conjugate of claim 1 that comprises at least one ligand selected from the group consisting of 38743, 156957, 127947, 73735, 55573, 41066, 11891, 63865, 408860, 362639, 36914, 23895, and 403374.

4. The ligand conjugate of claim 1 that comprises at least one ligand selected from the group consisting of 93033, 80807, 25368, 25678, 60239, 75866, 87504, 331931, 20586, 403374, 8481, and 5856.

5. The ligand conjugate of claim 1 that comprises at least one ligand selected from the group consisting of 16631, 40614, 68971, 78623, 81750, 401077, 408734, 303800, 75846, 638134, 70980, 89720, 25678, 215276, 16162 and 60239.

6. The ligand conjugate of claim 1 that comprises at least one ligand selected from the group consisting of 68982; 75866, 148832, 601359 and 142446.

7. The ligand conjugate of claim 1 that comprises at least one ligand selected from the group consisting of 75866, 87504, 25678, 40614, 134137, 7436, 117922, 144958, 68982, and 75846.

8. The ligand conjugate of claim 1 that is covalently attached to or non-covalently associated with an effector selected from the group consisting of biotin, avidin, avidin analog, antibody, protein, peptide, and lectin; or another effector.

9. The ligand conjugate of claim 1 that is covalently attached to or non-covalently associated with a carrier selected from the group consisting of a dendrimer, nanoparticle, a liposome, and a polymer; or another carrier.

10. A composition comprising at least one ligand conjugate according to claim 1 and a pharmaceutically acceptable carrier or excipient.

11. A ligand conjugate comprising at least two ligands that each bind to CD81 and when bound inhibit the attachment of HCV to CD81 and optionally a spacer or linker between the at least two molecules.

12. The ligand conjugate of claim 11 that is selected from the group consisting of 25678-lys-lys-75846, 40614-lys-lys-75846, 117922-lys-lys-75866, 75866-lys-lys-68982, 75866-lys-lys-144958, 40614-lys-lys-25678 and 40614-lys-25678-lys-75846.

13. The ligand conjugate of claim 11 that comprises a chemical linker selected from the group consisting of a chemical bond, a bivalent hydrocarbon radical, a multivalent hydrocarbon radical, a bivalent hydrocarbon radical containing at least one heteroatom, a multivalent hydrocarbon radical containing at least one heteroatom, and a multivalent radical containing oxygen, nitrogen or sulfur.

14. The ligand conjugate of claim 11 that comprises a chemical linker that is a peptide or peptide analog, a carbohydrate or carbohydrate analog, a sugar or sugar analog, nucleic acid or nucleic acid analog, or a dendrimer.

15. The ligand conjugate of claim 11 that is covalently attached to or non-covalently associated with an effector selected from the group consisting of biotin, avidin, avidin analog, antibody, protein, peptide, and lectin; or another effector.

16. The ligand conjugate of claim 11 that is covalently attached to or non-covalently associated with a carrier selected from the group consisting of a dendrimer, nanoparticle, a liposome, and a polymer; or another carrier.

17. A composition comprising at least one ligand conjugate according to claim 11 and a pharmaceutically acceptable carrier or excipient.

18. A method for modulating a biological activity of CD81 or an activity mediated by or through CD81 comprising contacting CD81 or a cell having CD81 with the ligand conjugate of claim 1.

19. A method for inhibiting the attachment of a pathogen that binds to CD81 to a cell having CD81 comprising contacting said cell with the ligand conjugate of claim 1.

20. The method of claim 19, wherein said pathogen is Hepatitis C virus (HCV).


SEQUENCE LISTING (4 sequences)

SEQ ID NO: 1 - protein, 4871 aa, Hepatitis C virus
  MISC_FEATURE (13011)..(): C4MR37
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNVSRCWVAITPTVATRDGKLPTTQLRRHIDLLVGSATLCSALYVGDLCGSVFLVGQLFTFSPRRHWTTQGCNCSIYPGHITGHRMAWDMMMNWSPTTALVVAQVLRIPQAILDMIAGAHWGVLAGIAYFSMVGNWAKVLVVLLLFAGVDAKTHVTGGSQARTTRDFADFFSPGPKQNIQLINTNGSWHINSTALNCNDSLNTGWVAGLFYYYKFNSSGCPERMASCRLLTDFDQGWGPISYTNGSGPDQRPYCWHYPPRPCGIVPAKSVCGPVYCFTPSPVVVGTTDRSGAPTYNWGENDTDVFVLNNTRPPLGNWFGCTWMNSTGFTKVCGAPPCVIGGVGNNTLHCPTDCFRKHPEATYSRCGSGPWITPRCLVDYPYRLWHYPCTINYTTFKVRMYVGGVEHRLEAACNWTRGERCDLEDRDRSELSPLLLSTTQWQVLPCSFTTLPALSTGLIHLHQNIVDVQYLYGVGSSIASWAIKWDYVVLLFLLLADARVCSCLWMMLLISQVEAALENLVILNAASLAGTHGLVSFLVFFCFAWYLKGKWVPGAVYAFYGMWPLLLLLLALPQRAYALDTEVAASCGGVVLVGLMALTLSPYYKRYISWCFWWLQYFLTRVEAQLHVWVPPLNVRGGRDAVILLTSALHPALVFDITKLLLAIFGPLWILQASLLKVPYFVRVQGLLRICALARKVVGGHYVQMVIIKLGALTGTYIYNHLTPLRDWAHNGLRDLAVAVEPVVFSPMETKLITWGADTAACGDIINGLPVSARRGQEILLGPADGMVSKGWRLLAPITAYAQQTRGLLGCIITSLTGRDKNQVEGEVQIVSTAAQTFLATCINGVCWTVYHGAGTRTIASSKGPVIQMYTNVDKDLVGWPAPQGARSLTPCACGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVPQSFQVAHLHAPTGSGKSTKVPAAYAAQGYKVLVLNPSVAATLGFGSYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGVNAVAYYRGLDVSVIPTSGDVVVVATDALMTGYTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVITPAVQTNWQKIEAFWAKHMWNFISGIQYLAGLSTLPGNPAIASLMAFTAAVTSPLTTSQTLLFNILGGWVAAQLAAPGAATAFVGAGLAGAAIGSVGLGKVLVDILAGYGAGVAMSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNASRCWVAMSPTVATRDGRLPTTQLRRHIDLLVGSATLCSALYVGDLCGSVFLVGQLFTFSPRRHWTTQGCNCSIYPGHITGHRMAWDMMMNWSPTAALVVAQLLRIPQAILDMIAGAHWGVLAGVAYFSMVGNWAKVLVVLLLFAGVDAQTHITGGSTAHAAAGLANLFSMGPKQDIRLINTNGSWHINRTALNCNESLNTGWVAGLLYYNKFNSSGCSERLASCRPLTDFAQGWGPISYANGSGPDQRPYCWHYPPRPCGIVPAKSVCGPVYCFTPSPVVVGTTDRSGAPTYSWGENETDVFILNNTRPPLGNWFGCTWMNSSGFTKVCGAPPCVIGGVGNNTLRCPTDCFRKHPEATYSRCGSGPWITPRCLVNYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWDYVVLLFLLLADARVCSCLWMMLLISQAEAALENLVVLNAASLAGTHGLVPFLVFFCFAWYLKGKWAPGAVYAFYGMWPFLLLLLALPHRAYALDTEVAASCGGVILAGLMALTLSPYYKRYISWCLWWLQYFLTRVEAQLHEWVPPLDVRGGRDAVILLMCVVHSTLVFDITKLLLAVLGPLWVLQASLLKVPYFVRVQGLLRICALARKMAGGHYVQMAIIKLGALTGTYVYNHLTPLRDWAHNGLRDLAVAVEPVVFSTMETKLITWGADTAACGDIINGLPVSARRGREILLGPADGMVSKGWRLLAPITAYAQQTRGLLGCIITSLTGRDKNQVEGEIQIVSTAAQTFLATCINGVCWTVYHGAGTRTIASPKGPVIQMYTNVDKDLVGWPAPQGARSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNVRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCNTCVTQTVDFSLDPTFTIETTTLPQDAVSRTQRRGRTGRGKPGIYRFVALGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLNRLKPTLHGPTPLLYRLGAVQNEVTLTHPITKYIMTCMSADLEIVTSTWVLVGGVLAALAAYCLSTGCVVIVGRIVLSGKPAIIPDREVLYREFDEMEECSQHLPYIEQGMALAEQFKQKALGLLQTASRQAEVITPTVQTNWQKLEAFWAKHMWNFISGIQYLAGLSTLPGNPAIASLMAFTAAVTSPLTTSQTLLFNILGGWVAAQLAAPGAATAFVGAGLAGAAIGGVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAATRVTAILSSLTVTQLLRRLHQWISSECTTPCSGSWLRDVWDWICEVLSDFKTWLKAKLVPQLPGIPFVSCQRGYKGVWRGDGIMHTRCHCGAEITGHVKNGTMRIVGPRTCRNMWSGTFPINAYTTGPCTPLPAPNYTFALWRVSAEEYVEIRRVGDFHYITGMTTDNLKCPCQVPSPEFFTELDGVRLHRFAPPCKPLLREEVSFRVGLHEYPVGSQLPCEPELDVAVLTSMLTDPSHITAEAAGRRLARGSPPSEASSSASQLSAPSLKATCTTNHDSPDAELIEANLLWRQEMGGNITRVESENKVVILDSFDPLVAEEDEREISVPAEILRKSRRFAPALPIWARPDYNPPLIETWKKPDYEPPVVHGCPLPPPQSPPVPPPRKKRTVVLTESTLPTALAELATKSFGSSSTSGITGDNTTTSSESAPSGCSPDSDAESFSSMPPLEGEPGDPDLSDGSWSTVSSGADAEDVVCCSMSYSWTGALVTPCAAEEQKLPINALSNSLLRHHNLVYSTTSRSACQRQKKVTFDRQQVLDSHYQDVLKEVKAAASKVKANLLSVEEACSLTPPHSARSKFGYGAKDVRCHARKAVNHINSVWKDLLEDSVTPIDTTIMAKNEVFCVQPEKGGRKPARLIVFPDLGVRVCEKMALYDVVSKLPVAVMGSSYGFQYSPGQRVEFLVQAWKSKKTPMGFSYDTRCFDSTVTENDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGRRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGQLDLSGWFTAGYSGGDIYHSVSHARPRWFWFCLLLLAAGVGIYLFPNR

SEQ ID NO: 2 - protein, 236 aa, Homo sapiens
  MISC_FEATURE (1)..(236): CD81 antigen isoform 1
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 3 - protein, 90 aa, Homo sapiens
  MISC_FEATURE (1)..(90): PDB 1G8Q_A.  Chain A, Crystal Structure Of Human Cd81 Extracellular Domain, A Receptor For Hepatitis C Virus
FVNKDQIAKDVKQFYDQALQQAVVDDDANNAKAVVKTFHETLDCCGSSTLTALTTSVLKNNLCPSGSNIISNLFKEDCHQKIDDLFSGKH

SEQ ID NO: 4 - protein, 90 aa, Homo sapiens; identical to SEQ ID NO: 3
  MISC_FEATURE (1)..(90): PDB 1G8Q_B.  Chain B, Crystal Structure Of Human Cd81 Extracellular Domain, A Receptor For Hepatitis C Virus